(19) United States
(12) Patent Application Publication
(10) Pub. No.: US 2022/0154209 A1
(43) Pub. Date: May 19, 2022

(54) PV-DELETED BOVINE ADENOVIRUS

(71) Applicant: University of Saskatchewan, Saskatoon (CA)

(72) Inventors: Suresh K. TIKOO, Saskatoon (CA); Xin ZHAO, Saskatoon (CA)

(21) Appl. No.: 17/392,164

(22) Filed: Aug. 2, 2021

Related U.S. Application Data
(63) Continuation of application No. 16/312,869, filed on Dec. 21, 2018, now abandoned.
(60) Provisional application No. 62/354,639, filed on Jun. 24, 2016.

(51) Int. Cl. C12N 15/86; A61K 35/761; A61K 39/00; A61K 39/02; A61K 39/12

(52) U.S. Cl. CPC C12N 15/86; A61K 35/761; A61K 39/0011; A61K 39/02; A61K 39/12; C12N 2710/10343; A61K 2039/5258; C12N 2710/10321; C12N 2710/10323; C12N 2710/10332; C12N 2710/10334; A61K 2039/5256

(57) ABSTRACT

The present application provided defective bovine adenovirus (BAV) vectors that lack pV function. Cell lines and methods of preparing such vectors are provided. In addition, the invention provides methods of treating a disease or disorder with a defective BAV lacking pV function as well as vaccine comprising a defective BAV lacking pV function.

### CROSS-REFERENCE TO RELATED APPLICATIONS

This application is a continuation of U.S. patent application Ser. No. 16/312,869, filed Dec. 21, 2018, which is a National Phase application under 35 U.S.C § 371 of International Application No. PCT/IB2017/000959 filed Jun. 23, 2017, which claims priority to U.S. Provisional Patent Application No. 62/354,639 filed Jun. 24, 2016, the disclosures of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 293102003840SEQLIST.txt, date recorded: Jun. 23, 2017, size: 56 KB).

### FIELD OF THE INVENTION

The present invention relates to bovine adenovirus (BAV) vectors with a deletion in pV and methods of making and using BAV vectors.

### BACKGROUND

Adenoviruses are non-enveloped icosahedral particles of 70 to 100 nM in diameter (Home et al., 1959, J. of Mol. Bio. 1:84-IN15; Thompson et al., 1981, The Canadian Veterinary Journal, 22; 68-71), which infect mammals, birds (Chiocca et al., 1996, J. Virol. 70:2939-2949), reptiles (Benko et al., 2002, J. Virol. 76:10056-10059), frogs (Davison et al., 2000, J. Gen. Virol. 81, 2431-2439) and fish (Kovacs et al., 2003, Virus Res. 98:27-34). Despite similarity in genome organization with human adenovirus (HAdV)-5, BAV-3 appears to possess certain distinct features (Bangari and Mittal, 2006, Vaccine 24:849-862; Idamakanti et al., 1999, Virology 256:351-359; Reddy et al., 1998 J. Virol. 72:1394-1402; Xing and Tikoo, 2006, J. Gen. Virol. 87:3539-3544; Xing and Tikoo, 2007, Virus Res. 130, 315-320; Xing et al., 2003, J. Gen. Virol. 84, 2947-2956) including organization of late (L) transcriptional unit into seven (L1-L7) regions (Reddy et al., 1998, J. Virol. 72:1394-1402).

Bovine adenovirus 3 contains a genome of 34,446 bp long organized into early (E), intermediate (I) and late (L) regions (Reddy et al., 1998, J. Virol. 72:1394-1402). Earlier, we reported that the core protein pVII encoded by L1 region of BAV-3 localizes to the mitochondria using a mitochondrial localization signal, and interferes with apoptosis by altering some mitochondrial functions in infected cells (Anand et al., 2014, J. Gen. Virol. 95:442-452). Recently, we reported that conserved regions of pVIII encoded by L6 region contain motifs involved in nuclear localization or packaging in mature virions (Ayalew et al., 2014, J. Gen. Virol. 95, 1743-1754). Similarly, conserved leucines (Kulshreshtha et al., 2015, Virology 483:174-184) and conserved arginines (Kulshreshtha et al., 2014, PloS 1 9:e101216) of 33K protein encoded by L6 region appeared important in binding and the activation of major late promoter, and in nuclear transport of 33K and BAV-3 replication, respectively.

Members of Mastadenovirus genus including human adenovirus (HAdV) infect mammals and encode unique proteins including pIX and pV (Davison et al., 2003, J. Gen. Virol. 84:2895-2908). The L2 region of HAdV-5 encodes a minor capsid protein named pV, which appears to associate with viral genome and bridge the core and the capsid proteins (Chatterjee et al., 1985, J. Virol. 55:379-386; Lehmberg et al., 1999, J. Chromatography. B, Biomedical sciences and applications 732:411-423; Matthews and Russell, 1998, J. Gen. Virol. 79:1677-1685; Vayda et al., 1983; Nucleic Acids Res 11, 441-460). Deletion of pV appears to be essential for virus replication in primary cells but not in cancerous cells (Ugai et al., 2007, J. Mol. Bio. 366:1142-1160). Protein V mainly localizes to the nucleolus utilizing a transportin dependent pathway (Hindley et al., 2007, J. Gen. Virol. 88:3244-3248) and over expression of pV redistributes nucleolin and nucleophosmin to the cytoplasm (Matthews, 2001, J. Virology 75:1031-1038). Additional investigations have revealed that pV promotes viral assembly through nucleophosmin 1 (Ugai et al., 2012, Virology 432:283-295) and is essential for virus replication in primary but not in cancer cells (Ugai et al., 2007, J. Gen. Virol. 79:1677-1685).

Though positional homologs are encoded by HAdV-5 and BAV-3, the structure and function of the homologous proteins may always not be similar (Anand et al., 2014, J. Gen. Virol. 95:442-452; Kulshreshtha et al., 2004, Virology 323:59-69; Li et al., 2009, Virology 392, 162-168; Reddy et al., 1998, J. Virol. 72:1394-1402). Recently, we demonstrated that unlike HAdV-5, bovine adenovirus-3 protease cleaves 100K protein, which is required for the nuclear transport in the infected cells but not for the virus replication (Makadiya et al., 2015, J. Gen. Virol. 96:2749-2763).

The L2 region of BAV-3, a member of Mastadenovirus genus, encodes pV protein of 423 amino acids, which shows 40.9% homology to pV encoded by HAdV-2 (Reddy et al., 1998 J. Virol. 72:1394-1402) and 28%-41% homology to pV proteins of other Mastadenoviruses.

Bovine adenovirus is described in WO 95/16048, WO 98/59063, WO 00/26395, WO 01/92547.

All references cited herein, including patent applications and publications, are incorporated by reference in their entirety.

### BRIEF SUMMARY OF THE INVENTION

In some aspects, the invention provides a defective bovine adenovirus (BAV) vector comprising inverted terminal repeat sequences and BAV packaging sequences, wherein the BAV vector lacks pV functions. In some embodiments, the BAV vector comprises one or more modifications of the nucleic acid encoding pV wherein the pV lacks nuclear localization functions and/or nucleolar localization functions.

In some embodiments, the defective BAV vector comprises a deletion of part or all of the coding region for pV. In some embodiments, the BAV vector comprises a deletion of all of the coding region for pV. In some embodiments, the BAV vector comprises a deletion corresponding to nucleotides 15068 to 16299 of SEQ ID NO:1. In some embodiments, the BAV vector comprises a deletion of nucleotides encoding amino acid residues 1-423 of the pV set forth in SEQ ID NO:2. In some embodiments, the BAV vector comprises a deletion of nucleotides encoding amino acid residues 21-50 and 380-389 of the pV set forth in SEQ ID NO:2. In some embodiments, the BAV vector comprises a deletion of nucleotides encoding amino acid residues 21-50, 190-210 and 380-389 of the pV set forth in SEQ ID NO:2. In some embodiments, the BAV vector comprises a deletion of nucleotides encoding amino acid residues 21-50 and 380-423 of the pV set forth in SEQ ID NO:2. In some embodiments, the BAV vector comprises a deletion of nucleotides encoding amino acid residues 21-50, 190-210 and 380-423 of the pV set forth in SEQ ID NO:2. In some embodiments, the BAV vector comprises a deletion of nucleotides encoding amino acid residues 21-50, 190-210 and 323-423 of the pV set forth in SEQ ID NO:2. In some embodiments, the BAV vector comprises a deletion of nucleotides encoding amino acid residues 21-50, 190-423 of the pV set forth in SEQ ID NO:2. In some embodiments, the BAV vector comprises a deletion of nucleotides encoding amino acid residues 21-50, 101-210 and 380-423 of the pV set forth in SEQ ID NO:2. In some embodiments, the BAV vector comprises a deletion of nucleotides encoding amino acid residues 3-100, 190-210 and 380-423 of the pV set forth in SEQ ID NO:2. In some embodiments, the BAV vector comprises a deletion of nucleotides encoding amino acid residues 21-50, 81-120, 190-210 and 380-423 of the pV set forth in SEQ ID NO:2. In some embodiments, the BAV vector comprises a deletion of nucleotides encoding amino acid residues 81-120, 190-210 and 380-423 of the pV set forth in SEQ ID NO:2. In some embodiments, the BAV vector comprises a deletion of nucleotides encoding amino acid residues 81-120, 190-210 and 390-423 of the pV set forth in SEQ ID NO:2. In some embodiments, the BAV vector comprises one or more substitutions of the nucleic acid encoding pV such that the BAV pV lacks nuclear localization functions and/or nucleolar localization functions. In some embodiments, substitution of the nucleic acid encoding pV results in the substitution of one or more of amino acid residues 21-50 or 380-389 of the pV set forth in SEQ ID NO:2. In some embodiments, the pV in the vector comprises the sequence set forth in SEQ ID NO:15.

In some embodiments of the above aspects and embodiments, the BAV vector further comprises a deletion of all or part of the E3 region. In some embodiments, the BAV vector further comprises nucleic acid encoding a heterologous transgene. In some embodiments, the nucleic acid encoding the heterologous transgene is located in the E3 region. In some embodiments, the heterologous transgene encodes a therapeutic polypeptide or a therapeutic nucleic acid. In some embodiments, the heterologous transgene encodes a coagulation factor, a hormone, a cytokine, a lymphokine, an oncogene product, a tumor suppressor, a cell receptor, a ligand for a cell receptor, a protease inhibitor, an antibody, a toxin, an immunogenic polypeptide, an antibody, a dystrophin, a cystic fibrosis transmembrane conductance regulator (CFTR), siRNA, mRNA, miRNA, lncRNA, tRNA, or shRNA. In some embodiments, the BAV vector is a BAV-3 vector.

In some aspects, the invention provides a recombinant bovine adenovirus (rBAV) particle, wherein the rBAV particle comprises a rBAV genome comprising inverted terminal repeat sequences and BAV packaging sequences, wherein the BAV genome lacks pV functions. In some embodiments, the rBAV genome comprises one or more modifications of the nucleic acid encoding pV wherein the pV lacks nuclear localization functions and/or nucleolar localization functions.

In some embodiments of the above aspects and embodiments, the rBAV genome comprises a deletion of part or all of the coding region for pV. In some embodiments, the rBAV genome comprises a deletion or all of the coding region for pV. In some embodiments, the rBAV genome comprises a deletion corresponding to nucleotides 15068 to 16299 of SEQ ID NO:1. In some embodiments, the rBAV genome comprises a deletion of nucleotides encoding amino acid residues 1-423 of the pV set forth in SEQ ID NO:2. In some embodiments, the rBAV genome comprises a deletion of nucleotides encoding amino acid residues 21-50 and 380-389 of the pV set forth in SEQ ID NO:2. In some embodiments, the rBAV genome comprises a deletion of nucleotides encoding amino acid residues 21-50, 190-210 and 380-389 of the pV set forth in SEQ ID NO:2. In some embodiments, the rBAV genome comprises a deletion of nucleotides encoding amino acid residues 21-50 and 380-423 of the pV set forth in SEQ ID NO:2. In some embodiments, the rBAV genome comprises a deletion of nucleotides encoding amino acid residues 21-50, 190-210 and 380-423 of the pV set forth in SEQ ID NO:2. In some embodiments, the rBAV genome comprises a deletion of nucleotides encoding amino acid residues 21-50, 190-210 and 323-423 of the pV set forth in SEQ ID NO:2. In some embodiments, the rBAV genome comprises a deletion of nucleotides encoding amino acid residues 21-50, 190-423 of the pV set forth in SEQ ID NO:2. In some embodiments, the rBAV genome comprises a deletion of nucleotides encoding amino acid residues 21-50, 101-210 and 380-423 of the pV set forth in SEQ ID NO:2. In some embodiments, the rBAV genome comprises a deletion of nucleotides encoding amino acid residues 3-100, 190-210 and 380-423 of the pV set forth in SEQ ID NO:2. In some embodiments, the rBAV genome comprises a deletion of nucleotides encoding amino acid residues 21-50, 81-120, 190-210 and 380-423 of the pV set forth in SEQ ID NO:2. In some embodiments, the rBAV genome comprises a deletion of nucleotides encoding amino acid residues 81-120, 190-210 and 380-423 of the pV set forth in SEQ ID NO:2. In some embodiments, the rBAV genome comprises a deletion of nucleotides encoding amino acid residues 81-120, 190-210 and 390-423 of the pV set forth in SEQ ID NO:2. In some embodiments, the rBAV genome comprises one or more substitutions of the nucleic acid encoding pV such that the BAV pV lacks nuclear localization functions and/or nucleolar localization functions. In some embodiments, substitution of the nucleic acid encoding pV results in the substitution of one or more of amino acid residues 21-50 or 380-389 of the pV set forth in SEQ ID NO:2. In some embodiments, the pV of the rBAV genome comprises the sequence set forth in SEQ ID NO:15.

In some embodiments of the above aspects and embodiments, the rBAV particle of comprises a rBAV genome wherein the rBAV genome further comprises a deletion of all or part of the E3 region. In some embodiments, the BAV vector further comprises nucleic acid encoding a heterologous transgene. In some embodiments, the nucleic acid encoding the heterologous transgene is located in the E3 region. In some embodiments, the heterologous transgene encodes a therapeutic polypeptide or a therapeutic nucleic acid. In some embodiments, the heterologous transgene encodes a coagulation factor, a hormone, a cytokine, a lymphokine, an oncogene product, a tumor suppressor, a cell receptor, a ligand for a cell receptor, a protease inhibitor, an antibody, a toxin, an immunogenic polypeptide, an antibody, a dystrophin, a cystic fibrosis transmembrane conductance regulator (CFTR), siRNA, mRNA, miRNA, lncRNA, tRNA, or shRNA. In some embodiments, the rBAV genome is a BAV-3 vector.

In some aspects, the invention provides a vaccine comprising a bovine adenovirus (rBAV) particle, wherein the rBAV particle comprises a rBAV genome comprising inverted terminal repeat sequences, BAV packaging sequences, and nucleic acid encoding a heterologous antigen; wherein the BAV genome lacks pV functions. In some embodiments, the rBAV genome of the vaccine comprises one or more modifications of the nucleic acid encoding pV wherein the pV lacks nuclear localization functions and/or nucleolar localization functions.

In some embodiments, the rBAV genome of the vaccine comprises a deletion of part or all of the coding region for pV. In some embodiments, the rBAV genome comprises a deletion of all of the coding region for pV. In some embodiments, the rBAV genome comprises a deletion corresponding to nucleotides 15068 to 16299 of SEQ ID NO:1. In some embodiments, the rBAV genome comprises a deletion of nucleotides encoding amino acid residues 1-423 of the pV set forth in SEQ ID NO:2. In some embodiments, the rBAV genome comprises a deletion of nucleotides encoding amino acid residues 21-50 and 380-389 of the pV set forth in SEQ ID NO:2. In some embodiments, the rBAV genome comprises a deletion of nucleotides encoding amino acid residues 21-50, 190-210 and 380-389 of the pV set forth in SEQ ID NO:2. In some embodiments, the rBAV genome comprises a deletion of nucleotides encoding amino acid residues 21-50 and 380-423 of the pV set forth in SEQ ID NO:2. In some embodiments, the rBAV genome comprises a deletion of nucleotides encoding amino acid residues 21-50, 190-210 and 380-423 of the pV set forth in SEQ ID NO:2. In some embodiments, the rBAV genome comprises a deletion of nucleotides encoding amino acid residues 21-50, 190-210 and 323-423 of the pV set forth in SEQ ID NO:2. In some embodiments, the rBAV genome comprises a deletion of nucleotides encoding amino acid residues 21-50, 190-423 of the pV set forth in SEQ ID NO:2. In some embodiments, the rBAV genome comprises a deletion of nucleotides encoding amino acid residues 21-50, 101-210 and 380-423 of the pV set forth in SEQ ID NO:2. In some embodiments, the rBAV genome comprises a deletion of nucleotides encoding amino acid residues 3-100, 190-210 and 380-423 of the pV set forth in SEQ ID NO:2. In some embodiments, the rBAV genome comprises a deletion of nucleotides encoding amino acid residues 21-50, 81-120, 190-210 and 380-423 of the pV set forth in SEQ ID NO:2. In some embodiments, the rBAV genome comprises a deletion of nucleotides encoding amino acid residues 81-120, 190-210 and 380-423 of the pV set forth in SEQ ID NO:2. In some embodiments, the rBAV genome comprises a deletion of nucleotides encoding amino acid residues 81-120, 190-210 and 390-423 of the pV set forth in SEQ ID NO:2. In some embodiments, the rBAV genome comprises one or more substitutions of the nucleic acid encoding pV such that the BAV pV lacks nuclear localization functions and/or nucleolar localization functions. In some embodiments, substitution of the nucleic acid encoding pV results in the substitution of one or more of amino acid residues 21-50 or 380-389 of the pV set forth in SEQ ID NO:2. In some embodiments, the pV of the BAV genome of the vaccine comprises the sequence set forth in SEQ ID NO:15.

In some embodiments of the above aspects and embodiments, the rBAV genome of the vaccine further comprises a deletion of all or part of the E3 region. In some embodiments, the nucleic acid encoding the heterologous antigen is located in the E3 region. In some embodiments, the heterologous antigen is a viral antigen, a microbial antigen, a tumor antigen. In some embodiments, the rBAV is a rBAV-3 particle.

In some aspects, the invention provides a pharmaceutical composition comprising a defective BAV vector as described herein. In some aspects, the invention provides a pharmaceutical composition comprising a rBAV particle as described herein. In some aspects, the invention provides a pharmaceutical composition comprising a vaccine as described herein. In some embodiments, the pharmaceutical composition further comprises a pharmaceutically acceptable excipient.

In some aspects, the invention provides a mammalian cell comprising nucleic acid encoding a BAV pV, said cell is capable of providing BAV pV function. In some embodiments, the BAV pV is BAV-3 pV. In some embodiments, the cell comprises nucleic acid encoding the BAV pV of SEQ ID NO:X. In some embodiments, the nucleic acid encoding BAV pV is operably linked to a promoter. In some embodiments, the promoter is a CMV promoter. In some embodiments, the nucleic acid encoding BAV pV comprises the nucleotide sequence of SEQ ID NO:X. In some embodiments, the cell is derived from CRL cells. In some embodiments, the nucleic acid encoding BAV pV is stably integrated into the genome of the cell.

In some aspects, the invention provides a method for producing a defective BAV vector comprising introducing a BAV genome to the cell described above and culturing the cells under conditions where the defective BAV vector is produced, wherein the defective BAV vector lacks pV function. In some embodiments, the BAV vector comprises one or more modifications of the nucleic acid encoding pV wherein the pV lacks nuclear localization functions and/or nucleolar localization functions.

In some embodiments of the above methods, the BAV vector comprises a deletion of part or all of the coding region for pV. In some embodiments, the BAV vector comprises a deletion of all of the coding region for pV. In some embodiments, the BAV vector comprises a deletion corresponding to nucleotides 15068 to 16299 of SEQ ID NO:1. In some embodiments, the BAV vector comprises a deletion of nucleotides encoding amino acid residues 1-423 of the pV set forth in SEQ ID NO:2. In some embodiments, the BAV vector comprises a deletion of nucleotides encoding amino acid residues 21-50 and 380-389 of the pV set forth in SEQ ID NO:2. In some embodiments, the BAV vector comprises a deletion of nucleotides encoding amino acid residues 21-50, 190-210 and 380-389 of the pV set forth in SEQ ID NO:2. In some embodiments, the BAV vector comprises a deletion of nucleotides encoding amino acid residues 21-50 and 380-423 of the pV set forth in SEQ ID NO:2. In some embodiments, the BAV vector comprises a deletion of nucleotides encoding amino acid residues 21-50, 190-210 and 380-423 of the pV set forth in SEQ ID NO:2. In some embodiments, the BAV vector comprises a deletion of nucleotides encoding amino acid residues 21-50, 190-210 and 323-423 of the pV set forth in SEQ ID NO:2. In some embodiments, the BAV vector comprises a deletion of nucleotides encoding amino acid residues 21-50, 190-423 of the pV set forth in SEQ ID NO:2. In some embodiments, the BAV vector comprises a deletion of nucleotides encoding amino acid residues 21-50, 101-210 and 380-423 of the pV set forth in SEQ ID NO:2. In some embodiments, the BAV vector comprises a deletion of nucleotides encoding amino acid residues 3-100, 190-210 and 380-423 of the pV set forth in SEQ ID NO:2. In some embodiments, the BAV vector comprises a deletion of nucleotides encoding amino acid residues 21-50, 81-120, 190-210 and 380-423 of the pV set forth in SEQ ID NO:2. In some embodiments, the BAV vector comprises a deletion of nucleotides encoding amino acid residues 81-120, 190-210 and 380-423 of the pV set forth in SEQ ID NO:2. In some embodiments, the BAV vector comprises a deletion of nucleotides encoding amino acid residues 81-120, 190-210 and 390-423 of the pV set forth in SEQ ID NO:2. In some embodiments, the BAV vector comprises one or more substitutions of the nucleic acid encoding pV such that the BAV pV lacks nuclear localization functions and/or nucleolar localization functions. In some embodiments, substitution of the nucleic acid encoding pV results in the substitution of one or more of amino acid residues 21-50 or 380-389 of the pV set forth in SEQ ID NO:2. In some embodiments, the pV encoded by the BAV vector comprises the sequence set forth in SEQ ID NO:15.

In some embodiments of the above methods, the BAV vector further comprises a deletion of all or part of the E3 region. In some embodiments, the BAV vector further comprises nucleic acid encoding a heterologous transgene. In some embodiments, the nucleic acid encoding the heterologous transgene is located in the E3 region. In some embodiments, the heterologous transgene encodes a therapeutic polypeptide or a therapeutic nucleic acid. In some embodiments, the heterologous transgene encodes a coagulation factor, a hormone, a cytokine, a lymphokine, an oncogene product, a tumor suppressor, a cell receptor, a ligand for a cell receptor, a protease inhibitor, an antibody, a toxin, an immunogenic polypeptide, an antibody, a dystrophin, a cystic fibrosis transmembrane conductance regulator (CFTR), siRNA, mRNA, miRNA, lncRNA, tRNA, or shRNA. In some embodiments, the BAV vector is a BAV-3 vector. In some embodiments, the BAV vector is encapsulated in a BAV particle. In some embodiments the invention provides a defective BAV vector prepared by any of the above methods. In some embodiments, the invention provides a pharmaceutical composition comprising the defective BAV vector prepared by any of the above methods. In some embodiments, the pharmaceutical composition further comprises a pharmaceutically acceptable excipient.

In some aspects the invention provides a method for treating a disease or disorder in an individual in need thereof comprising administering any of the pharmaceutical composition as described herein wherein the defective BAV vector of the rBAV particle comprises a heterologous transgene suitable for treating the disease or disorder. In some aspects, the invention provides a method for eliciting an immune response in an individual comprising administering any of the pharmaceutical composition described herein, wherein the defective BAV vector, the rBAV particle or the vaccine comprises a heterologous transgene encoding an antigen. In some embodiments, the pharmaceutical composition is administered in combination with another therapy. In some embodiments, the individual is a mammal. In some embodiments, the mammal is a cow, a pig, a sheep, a cat, a dog, a horse, a rabbit, a mouse, a rat, a hamster, a guinea pig, a non-human primate, or a human.

In some aspects the invention provides a use of any of the pharmaceutical compositions described herein for treating a disease or disorder in an individual in need thereof, wherein the defective BAV vector of the rBAV particle comprises a heterologous transgene suitable for treating the disease or disorder. In some aspects, the invention provides a use of any of the pharmaceutical composition described herein for eliciting an immune response in an individual, wherein the defective BAV vector, the rBAV particle or the vaccine comprises a heterologous transgene encoding an antigen. In some aspects, the invention provides a use of any of the pharmaceutical compositions described herein in the manufacture of a medicament for treating a disease or disorder in an individual in need thereof, wherein the defective BAV vector of the rBAV particle comprises a heterologous transgene suitable for treating the disease or disorder. In some aspects, the invention provides a use of any of the pharmaceutical composition described herein in the manufacture of a medicament for eliciting an immune response in an individual, wherein the defective BAV vector, the rBAV particle or the vaccine comprises a heterologous transgene encoding an antigen. In some embodiments, the pharmaceutical composition for administration in combination with another therapy. In some embodiments, the individual is a mammal. In some embodiments, the mammal is a cow, a pig, a sheep, a cat, a dog, a horse, a rabbit, a mouse, a rat, a hamster, a guinea pig, a non-human primate, or a human.

In some aspects, the invention provides a kit comprising any of the defective BAV vectors described herein. In some aspects, the invention provides a kit comprising any of the rBAV particles described herein. In some aspects, the invention provides a kit comprising any of the vaccines described herein. In some aspects, the invention provides a kit comprising any of the pharmaceutical formulations described herein. In some aspects, the invention provides a kit for use in any of the methods described herein, wherein the kit comprises any of the pharmaceutical compositions described herein. In some embodiments, any of the kits described above further comprises instructions for use. In some embodiments, any of the kits described above further comprises one or more of a buffer, a diluent, a filter, a needle, or a syringe.

### BRIEF DESCRIPTION OF THE DRAWINGS

FIG. 1A-C shows expression of pV. Proteins from BAV-3 infected MDBK cells (FIG. 1A) indicated plasmid DNA transfected cells (FIG. 1B) (lane 2 and 3) or mock infected/transfected cells were harvested at different time points, separated by SDS-PAGE and transferred to nitrocellulose membrane. The separated proteins were probed by Western blot using antipV serum. The position of the molecular weight marker (lane M) in kD was used for sizing the protein bands. (FIG. 1C) CRL cells were transfected with plasmid pDsRed. B23 DNA and infected by BAV-3 (FIG. 1D, panels a-d) or co-transfected with plasmid pcV and pDsRed. B23 DNAs (FIG. 1D, panels e-h) and fixed at 24 hours post-infection\transfection. The DsRed. B23 was visualized by direct fluorescence microscopy (panels b,f). BAV-3 pV was visualized by indirect immunofluorescence microscopy (panels c,g) using anti-pV antiserum and Alexa Fluor 488-conjugated goat anti-rabbit IgG. The nuclei were stained with DAPI.

FIG. 2A-C shows analysis of BAV-3 pV nucleolar localization signals. FIG. 2A shows schematic representation of BAV-3 pV. The thick black line represents BAV-3 pV. The numbers below represent amino acids of pV. Potential nuclear localization signal (NLS) and nucleolar localization sequences (NoLS1 and NoLS2) are depicted. NLS1 is SEQ ID NO:18, NLS2 is SEQ ID NO:19, NLS3 is SEQ ID NO:20: The name of the plasmid is depicted on the right. FIG. 2B shows schematic diagram represents mutant pV. Thick black lines represent pV gene, thin black lines represent the deleted regions. The name of the plasmid is depicted on the right. FIG. 2C shows sub cellular localization of pV mutants. Vero cells were transfected with those plasmids expressing pV and mutant pV genes individually and fixed with 4% formaldehyde at 48 h post-transfection. BAV-3 pV was visualized by indirect immunofluorescence using anti-pV antiserum and Alexa Fluor 488-conjugated goat anti-rabbit IgG (Jackson Immunoresearch). Nuclei were stained with DAPI and nucleoli were visualized with indirect immunostaining by using RPA194 Antibody (C-1) (Santa Cruz Biotechnology) and TRITC-conjugated goat anti-mouse IgG (Jackson Immunoresearch).

FIG. 3A-D shows mutation analysis of pV NoLs1. FIG. 3A shows schematic representation of BAV-3 pV depicting the amino acid sequence of NoLS1. The thick line represents BAV-3 pV gene. The thin line represents deleted region. The basic residue rich motifs (m1, m2, m3) are shown in different font size or indicated by small black bar. The numbers above represent amino acid of BAV-3 pV. The name of the plasmids is depicted on the right of the panel. pcV.d3: NoLS1 is SEQ ID NO:18, NoLS2 is SEQ ID NO:20. pcV.m1d3: NoLS1 is SEQ ID NO:29. pcV.m2d3: NoLS1 is SEQ ID NO:30. pcV.m3d3: NoLS1 is SEQ ID NO:31. pcV.m1m2d3: NoLS1 is SEQ ID NO:32. pcV.m1m3d3: NoLS1 is SEQ ID NO:33. pcV.m2m3d3: NoLS1 is SEQ ID NO:34. pcV.m1m2m3d3: NoLS1 is SEQ ID NO:35. FIG. 3B shows sub cellular localization of pV mutants. Vero cells were co-transfected with plasmid pDsRed. B23 and individual indicated plasmid DNAs. At 48 h post-transfection, cells were fixed with 4% formaldehyde. BAV-3 pV NoLS1 mutant proteins were visualized by indirect immunofluorescence microscopy using anti-pV antiserum and Alexa Fluor 488-conjugated goat anti-rabbit IgG (Jackson Immunoresearch). The DsRed. B23 was visualized by direct fluorescence microscopy. Nuclei were stained with DAPI. FIG. 3C shows schematic representation of fusion protein containing BAV-3 pV NoLSs. The white box represents BAV-3 pV nucleolar localization signals amino acids 21-50 or 380-389 (SEQ ID NO:36 or SEQ ID NO:37, respectively). The black box represents the EYFP gene. The numbers above represent amino acids of BAV-3 pV. (D) Sub cellular localization of fusion protein NoLs-EYFP. Vero cells were co-transfected with individual indicated plasmid expressing fusion proteins and pDsRed. B23 DNAs, and fixed with 4% formaldehyde at 48 h post-transfection. The DsRed. B23 (panel c, g, k) and EYFP (panel b, f, j) were visualized by direct fluorescence microscopy. Nuclei were stained with DAPI (panel a, e, i).

FIG. 4A-F shows analysis of BAV-3 pV nuclear localization signals. FIG. 4A, C show schematic representation of pV mutants. The thick black line represents BAV-3 pV gene. Thin black line represents the deleted regions. The numbers above represent amino acids of BAV-3 pV. The name of the plasmid is depicted on the right. FIG. 4B, D show sub cellular localization of pV mutants. Vero cells were transfected with individual indicated plasmid DNA and fixed with 4% formaldehyde at 48 h post-transfection. BAV-3 pV mutants were visualized by immunofluorescence using anti-pV antiserum and TRITC-conjugated goat anti-rabbit IgG (Jackson Immunoresearch). Nuclei were stained with DAPI. FIG. 4E shows schematic representation of GFP-βGal fusion protein containing BAV-3 pV NoLS1. The white box represents BAV-3 pV NoLS1 amino acids 21-50 (SEQ ID NO:36). The black box represents the fusion protein GFP-βGal. The numbers above represent amino acids of BAV-3 pV. The name of the plasmid is depicted on the right. FIG. 4F shows sub cellular localization of fusion protein NoLs1-GFP-βGal. Vero cells were transfected with those plasmids individually, and fixed with 4% formaldehyde at 48 h post-transfection. The GFP-βGal visualized with direct fluorescence microscopy. Nuclei were stained with DAPI.

FIG. 5A-D shows In vitro interaction of pV with transport receptors. FIGS. 5A and 5B shows in vitro interaction of pV with importin α3. In vitro synthesized and [35S]-labelled BAV-3 pV was incubated with purified GST fusion proteins (GST fused with importin α1, α3, α5, α7, β1, or transportin 3) or GST alone and pulled down with glutathione sepharose beads (GE Healthcare). FIG. 5C shows schematic representation of pV mutants. The thick black line represents BAV-3 pV gene. Thin broken line represents the deleted regions. The numbers above represent amino acids of BAV-3 pV. The name of the plasmid is depicted on the right. FIG. 5D shows in vitro interaction of pV mutants with importin α3. In vitro synthesized and [35S1-labelled BAV-3 pV mutants were incubated with purified GST-α3 or GST alone and pulled down with glutathione sepharose beads. Samples from FIG. 5A and FIG. 5C were separated by 10% SDS-PAGE and exposed to a phosphor screen. The exposed phosphor screen was visualized by Molecular Imager FX (Bio-Rad). 5% of the input radiolabelled pV mutants were used as control.

FIG. 6A-F shows L2 pV. FIG. 6A shows schematic representation of BAV-3 genomes. Dotted box represents BAV-3 genome, and thick black line represents pV sequence. The thin line depicts deleted regions. The arrows represent the direction of transcription. The amino acid numbers of pV are shown. The substituted amino acids (alanines\glycines) of NoLS1 are underlines and shown in italics (SEQ ID NO:35). E3 (early region 3); nucleolar localization signal (NoLS1 (SEQ ID NO:18), NoLs2 (SEQ ID NO:20)); CMV (human cytomegalovirus immediate early promoter); EYFP (enhanced yellow fluorescent protein). FIG. 6B shows fluorescent microscopy. The VIDO DT 1 or CRL. PV cells transfected with indicated plasmid DNAs were observed for appearance of green fluorescent cells and cytopathic effects. The numbers represent the day the observation was made after transfection. FIG. 6C shows restriction enzyme analysis of recombinant BAV-3 genome. The DNAs were extracted from MDBK or CRL. PV cells infected with BAV304a (lanes 2, 5, 6, and 8), BAV.pVdl (lane 1), BAV.pVm123 (lane 3), BAV.pVd3 (lane 4), and BAV.pVd1d3 (lanes 7 and 9) as described previously (Farina et al., 2001), digested with XbaI (lanes 1, 2, 3, 8, and 9) or Pst1 (lanes 4, 5, 6, and 7) and analyzed by agarose gel electrophoresis. FIG. 6D shows virus titer. Monolayers of MDBK cells were infected with BAV304a or recombinant BAV-3s. At different time points post infection, the cells were freeze-thawed and titrated on CRL. PV cells as described. Values represent averages of two independent repeats and error bars indicate the standard deviations. FIG. 6E shows Western Blot. Proteins from lysates of CRL or CRL.pV cells infected with BAV304a, BAV.pVd1, BAV.pVm123, BAV.pVd3 and BAV.pVd1d3 were separated by 10% SDS-PAGE, transferred to nitrocellulose membrane and probed by Western blot using anti-pV serum. The membrane was visualized by Odyssey® CLx Imaging System (LI-COR). FIG. 6F shows confocal microscopy. CRL cells were transfected with plasmid pDsRed. B23 DNA and infected with indicated mutant BAV-3s. Infected cells were fixed at 48 hrs postinfection. The DsRed. B23 was visualized by direct fluorescence microscopy (panels b,f). BAV-3 pV was visualized by indirect immunofluorescence microscopy using anti-pV serum and Alexa Fluor 647-conjugated goat anti-rabbit IgG. The nuclei were stained with DAPI.

FIG. 7A-B shows analysis of gene expression in mutant BAV-3 infected cells. FIG. 7A shows proteins from lysates of MDBK cells infected with indicated mutant BAV-3s. The proteins were separated by 10% SDS-PAGE, transferred to nitrocellulose and probed with protein specific antisera and Alexa Fluor 680 conjugated goat anti-rabbit antibody (Invitrogen). β-actin was used as a loading control and was detected using anti-β-actin monoclonal antibody (Sigma-Aldrich) and IRDye800 Conjugated goat anti-mouse antibody (Rockland). Protein names are depicted on the right of the panel. E (Early), L (Late), DBP (DNA binding protein). FIG. 7B shows protein quantification. The values were analyzed by using Odyssey® CLx Imaging System (LI-COR). Values represent the averages of two independent repeats and error bars indicate the standard deviations.

FIG. 8 shows structural protein incorporation assay. Structural proteins from the purified BAV304a (lane 1), BAV.pVd1 (lane 2), BAV.pVd1 (lane 3), BAV.pVd3 (lane4) or BAV.pVd1d3 (lane 5) grown in CRL cells and BAV.pV BAV.pVd1d3 (lane 6) grown in CRL.pV cells were separated by 10% SDS-PAGE, transferred to nitrocellulose and probed by Western blot using protein specific antisera. Protein bands were visualized by Odyssey® CLx Imaging System (LI-COR). Protein names are depicted on the right of the panel.

FIG. 9A-B shows transmission electron microscopic analysis. FIG. 9A shows viral assembly in infected cells. Uninfected MDBK cells (Panel 1, 2), MDBK cells infected with BAV.pVd1d2 (Panel 3, 4) or BAV304a (Panel 5, 6). The arrows depict higher magnification (60 000×) of the areas in the boxes. FIG. 9B shows negative staining of purified BAV304a (Panel 1, 2) and BAV.pVd1d2 (Panel 3, 4). The arrows depict higher magnification (120 000×) of the areas in the boxes.

FIG. 10A-F shows thermostability of the recombinant BAV-3s. FIG. 10A shows thermostability assay of the recombinant BAV-3s. 10⁵ TCID₅₀ of BAV304a, BAV.pVm123, BAV.pVd1 or BAV.pVd3 virions purified from CRL cells or 10⁵ TCID₅₀ of BAV.pVd1d3 virions purified from CRL.pV cells were incubated at −80° C., 20° C., 4° C., 25° C. or 37° C. for 3 days, and the residual viral infectivity was determined with TCID₅₀ on CRL.pV cells. Values represent averages of two independent repeats and error bars indicate the standard deviations. FIG. 10B-F shows thermostability assay of the recombinant BAV-3s. 10⁵ TCID₅₀ of BAV304a, BAV.pVm123, BAV.pVd1 or BAV.pVd3 virions purified from CRL cell or 10⁵ TCID₅₀ of BAV.pVd1d3 virions purified from CRL.pV cells were incubated at −80° C., 4° C. or 37° C. for 0, 1, 3 or 7 days. The residual viral infectivity was determined with TCID₅₀ using CRL.pV cells. Values represent averages of two independent repeats and error bars indicate the standard deviations.

FIG. 11A-B shows isolation of pV deleted BAV-3. FIG. 11A shows a schematic diagram of indicated plasmid DNA. Thick black box represents BAV-3 genomic DNA. Dotted line represents deleted region. Thin lines represent plasmid DNA. The human cytomegalovirus immediate early promoter (CMV), enhanced yellow fluorescent protein gene (EYFP), ampicillin resistance gene (Amp), early region 3 (E3) and pV location is depicted. FIG. 11B shows direct fluorescence. Monolayer of VIDO DT1 cells (Du and Tikoo, 2010) were transfected with 7.5 μg indicated plasmid DNA and visualized for the expression of EYFP and development of cytopathic effects using fluorescent microscope TCS SP5 (Leica).

FIG. 12A-B shows analysis of pV expression in CRL.pV cells. FIG. 12A shows proteins from the cell lysates of CRL.pV cells clone 1 (lane 1) and clone 2 (lane 2), BAV-3 infected CRL cells (lane 3) and mock infected CRL cells (lane 4) were separated by 10% SDS-PAGE, transferred to nitrocellulose membrane and probed in Western blot by using rabbit anti-pV serum and Alexa Fluor 680 conjugated antibodies goat anti-rabbit antibody (Invitrogen). The position of the molecular weight in kDa is shown on the left of the panel. The molecular weight in kDa of observed protein is shown on the right of the panel. FIG. 12B shows monolayers of CRL.pV (clone 1 or 2) or CRL cells were fixed with 4% paraformaldehyde and visualized by indirect immunostaining with rabbit anti-pV serum followed by TRITC-conjugated goat anti-rabbit IgG using confocal microscope TCS SP5 (Leica). The nuclei were stained by DAPI.

FIG. 13A-F shows construction and identification of BAV.dV. FIG. 13A shows a schematic diagram of plasmid pUC304A.dV DNA as described in FIG. 11A. FIG. 13B shows direct fluorescence. Monolayer of CRL.pV cells were transfected with 7.5 μg pUC304A.dV plasmid DNA and visualized for the expression of EYFP and development of cytopathic effects using fluorescent microscope TCS SP5 (Leica). FIG. 13C shows restriction enzyme analysis of BAV-3 genomes. The viral DNA was extracted from CRL cells infected with BAV304a (lane 1) or BAV.dV (lane 2), digested with KpnI and analyzed by agarose gel electrophoresis. Lane M, GeneRuler 1 kb DNA ladder (Thermo Fisher Scientific) was used for sizing the viral DNA fragments. Diagnostic bands are indicated with white arrows. Sizes of markers are shown on the left of the panel. FIG. 13D shows western blot. Proteins from the lysates of BAV304a infected CRL cells (lane 1), BAV.dV infected CRL cells (lane 2) or uninfected CRL.pV cells (lane 3) were separated by SDS-PAGE, transferred to nitrocellulose membrane and probed in Western blot by using rabbit anti-pV anti-serum and Alexa Fluor 680 conjugated goat anti-rabbit antibody (Invitrogen). The position of the molecular weight in kDa is shown on the left of the panel. The molecular weight in kDa of the observed protein is shown on the right of the panel. FIG. 13E shows CsCl gradient purification. The lysates of CRL.pV or CRL infected with BAV.dV were separated by centrifugation through continuous CsCl gradient and centrifuge tubes were photographed. FIG. 13F shows virus growth. Confluent monolayers of CRL cells were infected with BAV304a or BAV.dV at a MOI of 2. At different times post infection, the cell pellets were collected, freeze-thawed, and virus was titrated on CRL.pV cells as described previously (Ugai et al., 2007). Each value represents the average of two independent repeats and error bars indicate the standard deviations.

FIG. 14A-C Analysis of viral protein expression in BAV.dV infected cells. FIG. 14A shows western blots. Proteins from the lysates of CRL cells were separated by SDS-PAGE, transferred to nitrocellulose membranes and probed by Western blot using anti-DBP (Zhou et al., 2001), anti-pVII (Paterson et al., 2012), anti-pV (Kulshreshtha et al., 2004), anti-pX (Paterson, 2010), anti-Hexon (Kulshreshtha et al., 2004) and anti-100K (Makadiya et al., 2015) sera followed by Alexa Fluor 680 conjugated goat anti-rabbit antibody (Invitrogen). (3-actin was detected by Western blot using mouse anti-β-actin monoclonal antibody (Sigma-Aldrich) followed by IRDye800 Conjugated goat anti-mouse antibody (Rockland). The name of the proteins is depicted on the right of the panel. DBP (DNA binding protein). Early (E), Late (L). FIG. 14B shows quantification of the blot. The results were analyzed by using Odyssey Infrared Imaging System. Values represent averages from two independent repeats and error bars indicate the standard deviations. FIG. 14C shows DNA replication. Viral DNAs were extracted at indicated times post-infection with BAV304a (lane 2, 4, 6) or BAV.dV (lanes3, 5, 7) and digested with Bmt1. The specific bands are indicated by white arrows. The sizes of markers (M) are depicted on left and right of the panel.

FIG. 15A-B shows Analysis of viral protein incorporation in purified virions. FIG. 15A shows proteins from the purified BAV304a grown in CRL cells (lane 1), BAV.dV grown in CRL cells (lane 2), BAV.dV grown in CRL.pV cells were separated by 10% SDS-PAGE, transferred to nitrocellulose and probed in Western blot using protein specific antisera. FIG. 15B shows proteins from purified BAV304a grown in CRL cells (lanes 1), BAV.dV grown in CRL cells (lane 3), BAV.dV grown in CRL.pV cells (lane 6) or proteins from the lysates of CRL cells (lane 2, 4) infected with BAV304a (lane 2), BAV.dV (lane 4) and CRL.pV cells infected with BAV.dV (lane 6) were separated by 10% SDS-PAGE, transferred to nitrocellulose and probed in Western blot by anti-pVII serum (Paterson, 2010). Purified virus (PV); infected cell (IC).

FIG. 16A-B shows electron microscopic analysis. FIG. 16B shows uninfected (panel 1), BAV304a infected (panel 3) or BAV.dV infected (panel 5) CRL cells. The arrows depicted the enlargement of selected boxed region of panel 1 (panel 2), panel 3 (panel 4) and panel 5 (panel 6). FIG. 16B shows purified BAV304a (panel 1) or BAV.dV (panel 2). The arrows depicted the enlargement of selected boxed region of panel 1 (panel 2) and panel 3 (panel 4).

FIG. 17A-B shows thermostability of BAV-3. FIG. 17A shows viruses grown in CRL.pV cells. Purified virions (10⁵ TCID₅₀) grown in CRL.pV cells were incubated at various temperatures for 3 days (panel 1) and the residual viral infectivity was determined by titration on CRL.pV cells. Purified BAV304a (panel 2) or BAV.dV (panel 3) (10⁵ TCID₅₀) grown in CRL.pV cells were incubated at different temperatures for indicated periods of time and the residual viral infectivity was determined by titration on CRL.pV cells. FIG. 17B shows viruses grown in CRL cells. Purified virions (10⁴ TCID₅₀) grown in CRL cells were incubated at various temperatures for 3 days (panel 1) and the residual viral infectivity was determined by titration on CRL.pV cells. Purified BAV304a (panel 2) or BAV.dV (panel 3) (10⁴ TCID₅₀) grown in CRL cells were incubated at different temperatures for indicated periods of time and the residual viral infectivity was determined by titration on CRL.pV cells.

### DETAILED DESCRIPTION

The invention provides defective bovine adenovirus (BAV) vectors comprising inverted terminal repeat sequences and BAV packaging sequences, wherein the BAV vector lacks pV functions. In some embodiments, the BAV vector comprises one or more modifications of the nucleic acid encoding pV wherein the pV lacks nuclear localization functions and/or nucleolar localization functions. In some embodiments, the BAV vector comprises a deletion of part or all of the coding region for pV. Defective BAV vector genomes, comprising a modification that alters pV function, may be packaged into BAV capsids comprising native pV. Such encapsidated BAV vectors can infect cells but replicate its genome but cannot form infection BAV particles by virtue of the modification altering pV function. In some embodiments, the defective BAV vectors encode a heterologous transgene (e.g., an antigen). In some embodiments, the encapsidated BAV vectors are used to deliver a heterologous transgene to a cell; for example, to treat a disease or disorder (e.g., for gene therapy) or to elicit an immune response (e.g., a vaccine). Methods to produce defective BAV vectors are also contemplated.

### I. General Techniques

The techniques and procedures described or referenced herein are generally well understood and commonly employed using conventional methodology by those skilled in the art, such as, for example, the widely utilized methodologies described in Molecular Cloning: A Laboratory Manual (Sambrook et al., 4^{th} ed., Cold Spring Harbor Laboratory Press, Cold Spring Harbor, N.Y., 2012); Current Protocols in Molecular Biology (F. M. Ausubel, et al. eds., 2003); the series Methods in Enzymology (Academic Press, Inc.); PCR 2: A Practical Approach (M. J. MacPherson, B. D. Hames and G. R. Taylor eds., 1995); Antibodies, A Laboratory Manual (Harlow and Lane, eds., 1988); Culture of Animal Cells: A Manual of Basic Technique and Specialized Applications (R. I. Freshney, 6^{th} ed., J. Wiley and Sons, 2010); Oligonucleotide Synthesis (M. J. Gait, ed., 1984); Methods in Molecular Biology, Humana Press; Cell Biology: A Laboratory Notebook (J. E. Cellis, ed., Academic Press, 1998); Introduction to Cell and Tissue Culture (J. P. Mather and P. E. Roberts, Plenum Press, 1998); Cell and Tissue Culture: Laboratory Procedures (A. Doyle, J. B. Griffiths, and D. G. Newell, eds., J. Wiley and Sons, 1993-8); Handbook of Experimental Immunology (D. M. Weir and C. C. Blackwell, eds., 1996); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Ausubel et al., eds., J. Wiley and Sons, 2002); Immunobiology (C. A. Janeway et al., 2004); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane, Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 2011).

### II. Definitions

A “vector,” as used herein, refers to a recombinant plasmid or virus that comprises a nucleic acid to be delivered into a host cell, either in vitro or in vivo.

A “recombinant bovine adenoviral vector (rBAV vector)” refers to a polynucleotide vector comprising one or more heterologous transgene sequences (i.e., nucleic acid sequence not of BAV origin) that are flanked by BAV inverted terminal repeat sequences (ITRs). Such rBAV vectors can be replicated and packaged into infectious viral particles when present in a host cell where suitable BAV functions are provided (e.g., to complement essential viral functions impaired in the BAV vector (e.g., a pV region). An rBAV vector can be in any of a number of forms, including, but not limited to, plasmids, linear artificial chromosomes, complexed with lipids, encapsulated within liposomes, and encapsidated in a viral particle; for example, a BAV particle. A defective BAV vector can be packaged into a BAV virus capsid to generate a “recombinant bovine adenoviral particle (rBAV particle)”.

A “live virus” as used herein refers to a virus which is capable of producing identical progeny in tissue culture and inoculated animals, in contrast to a “killed virus.”

A “helper-free virus vector” is a vector that does not require a second virus or a cell line to supply something defective in the vector.

The term “polynucleotide” or “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be an oligodeoxynucleoside phosphoramidate (P—NH₂) or a mixed phosphoramidate-phosphodiester oligomer. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

As used herein, a “coding sequence” is a nucleic acid sequence which is transcribed and translated into a polypeptide when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, prokaryotic sequences, cDNA from eukaryotic mRNA, genomic DNA sequences from eukaryotic (e.g., mammalian) DNA, viral DNA, and even synthetic DNA sequences. A polyadenylation signal and transcription termination sequence will usually be located 3′ to the coding sequence.

A “promoter” or “promoter sequence” is a nucleic acid regulatory region capable of binding RNA polymerase in a cell and initiating transcription of a downstream (3′ direction) coding sequence. For purposes of defining the present invention, the promoter sequence is bound at the 3′ terminus by the translation start codon (ATG) of a coding sequence and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eukaryotic promoters will often. but not always, contain “TATA” boxes and “CAAT” boxes. Prokaryotic promoters contain Shine-Dalgarno sequences in addition to the −10 and −35 consensus sequences.

Nucleic acid “control sequences” refer collectively to promoter sequences, ribosome binding sites, splicing signals, polyadenylation signals, transcription termination sequences, upstream regulatory domains, enhancers, translational termination sequences and the like which collectively provide for the transcription and translation of a coding sequence in a host cell.

A coding sequence or sequence encoding is “operably linked to” or “under the control of” control sequences in a cell when RNA polymerase will bind the promoter sequence and transcribe the coding sequence into mRNA, which is then translated into the polypeptide encoded by the coding sequence.

A “host cell” is a cell which has been transformed, or is capable of transformation, by an exogenous DNA sequence.

A cell has been “transformed” by exogenous nucleic acid when such exogenous nucleic acid has been introduced inside the cell membrane. Exogenous nucleic acid may or may not be integrated (covalently linked) to chromosomal DNA making up the genome of the cell. In prokaryotes and yeasts, for example, the exogenous nucleic acid may be maintained on an episomal element, such as a plasmid. A stably transformed cell is one in which the exogenous nucleic acid has become integrated into the chromosome so that it is inherited by daughter cells through chromosome replication. For mammalian cells, this stability is demonstrated by the ability of the cell to establish cell lines or clones comprised of a population of daughter cell containing the exogenous nucleic acid.

“Heterologous” means derived from a genotypically distinct entity from that of the rest of the entity to which it is compared or into which it is introduced or incorporated. For example, a polynucleotide introduced by genetic engineering techniques into a different cell type is a heterologous polynucleotide (and, when expressed, can encode a heterologous transgene). Similarly, a cellular sequence (e.g., a gene or portion thereof) that is incorporated into a viral vector is a heterologous nucleotide sequence with respect to the vector.

The term “transgene” refers to a polynucleotide that is introduced into a cell and is capable of being transcribed into RNA and optionally, translated and/or expressed under appropriate conditions. In aspects, it confers a desired property to a cell into which it was introduced, or otherwise leads to a desired therapeutic or diagnostic outcome. In another aspect, it may be transcribed into a molecule that mediates RNA interference, such as siRNA.

Two polypeptide sequences are “substantially homologous” when at least about 80% (preferably at least about 90%. and most preferably at least about 95%) of the amino acids match over a defined length of the molecule.

Two nucleic acid sequences are “substantially homologous” when they are identical to or not differing in more than 40% of the nucleotides, preferably not more than about 30% of the nucleotides (i.e. at least about 70% homologous) more preferably about 20% of the nucleotides, and most preferably about 10% of the nucleotides.

“Percent (%) sequence identity” with respect to a reference polypeptide or nucleic acid sequence is defined as the percentage of amino acid residues or nucleotides in a candidate sequence that are identical with the amino acid residues or nucleotides in the reference polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various ways that are within the skill in the art, for instance, using publicly available computer software programs, for example, those described in Current Protocols in Molecular Biology (Ausubel et al., eds., 1987), Supp. 30, section 7.7.18, Table 7.7.1, and including BLAST, BLAST-2, ALIGN or Megalign (DNASTAR) software. A preferred alignment program is ALIGN Plus (Scientific and Educational Software, Pennsylvania). Those skilled in the art can determine appropriate parameters for measuring alignment, including any algorithms needed to achieve maximal alignment over the full length of the sequences being compared. For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. For purposes herein, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows: 100 times the fraction W/Z, where W is the number of nucleotides scored as identical matches by the sequence alignment program in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

An “isolated” molecule (e.g., nucleic acid or protein) or cell means it has been identified and separated and/or recovered from a component of its natural environment.

An “effective amount” is an amount sufficient to effect beneficial or desired results, including clinical results (e.g., amelioration of symptoms, achievement of clinical endpoints, and the like). An effective amount can be administered in one or more administrations. In terms of a disease state, an effective amount is an amount sufficient to ameliorate, stabilize, or delay development of a disease.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

As used herein, “treatment” is an approach for obtaining beneficial or desired clinical results. For purposes of this invention, beneficial or desired clinical results include, but are not limited to, alleviation of symptoms, diminishment of extent of disease, stabilized (e.g., not worsening) state of disease, preventing spread (e.g., metastasis) of disease, delay or slowing of disease progression, amelioration or palliation of the disease state, and remission (whether partial or total), whether detectable or undetectable. “Treatment” can also mean prolonging survival as compared to expected survival if not receiving treatment.

A “substantially pure” protein will be free of other proteins, preferably at least 10% homogeneous, more preferably 60% homogeneous, and most preferably 95% homogeneous.

An “antigen” refers to a molecule containing one or more epitopes that will stimulate a host's immune system to make a humoral and/or cellular antigen-specific response. The term is also used interchangeably with “immunogen.”

A “hapten” is a molecule containing one or more epitopes that does not stimulate a host's immune system to make a humoral or cellular response unless linked to a carrier.

The term “epitope” refers to the site on an antigen or hapten to which a specific antibody molecule binds or is recognized by T cells. The term is also used interchangeably with “antigenic determinant” or “antigenic determinant site.”

An “immunological response” to a composition or vaccine is the development in the host of a cellular and/or antibody-mediated immune response to the composition or vaccine of interest. Usually, such a response consists of the subject producing antibodies,

B cells, helper T cells, suppressor T cells, and/or cytotoxic T cells directed specifically to an antigen or antigens included in the composition or vaccine of interest.

The terms “immunogenic polypeptide” and “immunogenic amino acid sequence” refer to a polypeptide or amino acid sequence, respectively, which elicit antibodies that neutralize viral infectivity, and/or mediate antibody-complement or antibody-dependent cell cytotoxicity to provide protection of an immunized host. An “immunogenic polypeptide” as used herein, includes the full length (or near full length) sequence of the desired protein or an immunogenic fragment thereof.

By “immunogenic fragment” is meant a fragment of a polypeptide which includes one or more epitopes and thus elicits antibodies that neutralize viral infectivity, and/or mediates antibody-complement or antibody-dependent cell cytotoxicity to provide protection of an immunized host. Such fragments will usually be at least about 5 amino acids in length. and preferably at least about 10 to 15 amino acids in length. There is no critical upper limit to the length of the fragment, which could comprise nearly the full length of the protein sequence. or even a fusion protein comprising fragments of two or more of the antigens. The term “treatment” as used herein refers to treatment of a mammal, such as bovine or human or other mammal, either (i) the prevention of infection or reinfection (prophylaxis), or (ii) the reduction or elimination of symptoms of an infection. The vaccine comprises the recombinant BAV itself or recombinant antigen produced by recombinant BAV.

By “infectious” is meant having the capacity to deliver the viral genome into cells.

Reference to “about” a value or parameter herein includes (and describes) embodiments that are directed to that value or parameter per se. For example, description referring to “about X” includes description of “X.”

As used herein, the singular form of the articles “a,” “an,” and “the” includes plural references unless indicated otherwise.

It is understood that aspects and embodiments of the invention described herein include “comprising,” “consisting,” and/or “consisting essentially of” aspects and embodiments.

### III. BAV Vectors

The invention provides defective bovine adenovirus (BAV) vectors comprising inverted terminal repeat sequences and BAV packaging sequences, wherein the BAV vector lacks pV functions (e.g., less than about any of 50%, 40%, 30%, 20%, 10%, 5% or 1% native pV functions). In some embodiments, the BAV vector comprises one or more modifications of the nucleic acid encoding pV wherein the pV lacks nuclear localization functions and/or nucleolar localization functions. In some embodiments, the BAV vector comprises one or more modifications of the nucleic acid encoding pV wherein the pV has dimished nuclear localization functions and/or nucleolar localization functions (e.g., less than about any of 50%, 40%, 30%, 20%, 10%, 5% or 1% native pV nuclear and/or nucleolar localization functions). In some embodiments, the BAV vector comprises a deletion of part or all of the coding region for pV. Defective BAV vector genomes, comprising a modification that alters pV function, may be packaged into BAV capsids comprising native pV. Such encapsidated BAV vectors can infect cells but replicate its genome but cannot form infection BAV particles by virtue of the modification altering pV function.

The members of Mastadenovirus genus encode genus specific unique proteins including pIX and pV (Davison et al., 2003). Earlier work has suggested that HAdV-5 pV protein is essential for virus replication in primary cells and may participate in virus assembly (Ugai et al., 2012). Moreover, pV may act as a bridge between the core and the capsid proteins of HAdV-5 (Chatterjee et al., 1985; Lehmberg et al., 1999; Matthews and Russell, 1998; Vayda et al., 1983). The L2 region of BAV-3 encodes pV, which shows 28%-41% homology to pV encoded by other members of Mastadenovirus genus (Reddy et al., 1998). Recent reports suggest that positional homologs of proteins encoded by human and animal adenoviruses of Mastadenovirus genus may differ in their structure and function (Li et al., 2009, Makadiya et al., 2014).

The BAV pV appears essential for the replication of BAV304a as production of viable infectious BAV.dV required the isolation of helper cell line providing the pV protein in trans. Analysis of BAV.dV demonstrate no significant difference in the infectivity and DNA replication of mutant BAV.dV and BAV304a. Moreover, no affect is observed in the early gene expression in BAV.dV infected cells. Despite down regulation of some late protein expression, the capsid formation and the virus assembly appeared to occur in BAV.dV infected cells suggesting that pV may not be essential for virion assembly. Earlier report suggested that pV is essential for the replication of HAdV-5 in primary cells but not in cancer cells (Ugai et al., 2012). In contrast, BAV-3 pV appears essential for replication in primary CRL cells and continuous MDBK cells.

Although, BAV.dV does not appear to produce infectious progeny virions in CRL cells, the capsid formation and the virus assembly appears to occur in BAV.dV infected CRL cells as CsCl gradient analysis of BAV.dV infected CRL cells produced virions banding at CsCl gradient density consistent with the formation of mature virions. Moreover, the deletion of pV does not significantly affect the incorporation of other structural proteins. However, the analysis of these mature virions by TEM revealed that, compared to BAV304a, the capsids of BAV.dV do not appear icosahedral in shape and most of the capsids do not appear to be intact. Without being bound by theory, these results suggest that deletion of BAV.dV may not significantly alter the virus assembly, but instead make virion capsid more fragile leading to the detectable changes in virion morphology and infectivity.

The deletion of pV does not affect the expression of early gene product, e.g, namely DBP. However, the expression of late proteins particularly 100K, pX and pVII appear down regulated in BAV.dV infected cells, suggesting that pV may be involved in the regulation of late gene expression probably by acting on major late promoter (Leong et al., 1990). Similar results have been earlier reported for HAdV-5 pV (Ugai et al., 2007).

The production of infectious progeny adenovirus requires a maturation step involving the cleavage of capsid and core proteins by adenovirus protease (Anderson et al., 1973). However, the significance of cleavage of each precursor protein in determining the infectivity is not clear (Mangel and San Martin, 2014). Analysis of viral protein expression in BAV.dV infected cells revealed that deletion of pV did not significantly inhibit the cleavage of pVII. Similarly, analysis of purified BAV.dV demonstrated that mainly the cleaved form of pVII or pVIII could be detected in purified mature BAV.dV virions.

Unlike primary cells (Ugai et al., 2012), HAdV-5 pV is not required for virus replication and formation of infectious virus particles in cancer cells (Ugai et al., 2012). This is due to apparent thermostable mutations (G13E and R17I) in the less conserved region of core protein X/Mu, which compensate for the lack of pV (Ugai et al., 2007). Moreover, analysis of CsCl gradient purified pV deleted HAdV-5 grown in cancer cells show increased incorporation of protein X\Mu in mature virions. In contrast, BAV pV may be essential for the replication of BAV-3 CRL or MDBK cells. Despite conservation of arginine residue at amino acid 20 of BAV-3 pV (Ugai et al., 2007), analysis of DNA sequence of different clones of BAV.dV grown (different passages) in CRL or MDBK cells do not reveal any mutation in the core proteins X\Mu or pVII (data not shown).

Although adenovirus protein homologs are encoded by members of Mastadenovirus genus, recent reports have demonstrated the differences in the subcellular localization and function of homologous adenovirus proteins (Blanchette et al., 2013; Cheng et al., 2013; Stracker et al., 2005). Recently, we reported that 100K protein encoded by HAdV-5 and BAV-3 differ in sub cellular localization and protein function (Makadiya et al., 2015). Adenovirus pV is a Mastadenovirus genus specific minor capsid protein, which localizes to both the nucleus and the nucleolus in infected cells (Matthews, 2001). Although transportin appears necessary for the nucleolar localization of pV (Hindley et al., 2007), the molecular mechanism involved in the nucleolar localization is not known. The present study was designed to characterize BAV-3 pV protein, investigate the mechanism of nucleolar localization and determine its role in virus replication.

The BAV-3 pV encodes a protein of 423 amino acids, which is expressed as 55 kD protein, appears between 12-24 hrs post infection and could be detected till 48 hrs post BAV-3 infection. pV is almost exclusively detected in the nucleolus of the BAV-3 infected or transfected cells in the absence of any other viral protein.

Proteins localizing to nucleolus also localize to the nucleus and thus may contain either overlapping NLS\NoLS (Cheng et al., 2002; Sheng et al., 2004) or separate nonoverlapping signals for localizing to both the nucleus and the nucleolus (Cros et al., 2005; Ladd and Cooper, 2004). Amino acid sequence analysis of BAV-3 pV predicted three clusters of arginine-lysine rich sequences in both N-terminus (amino acid 21-50), central domain (amino acid 190-210) and C-terminus (amino acid 380-389) of pV with potential to act as NLS.

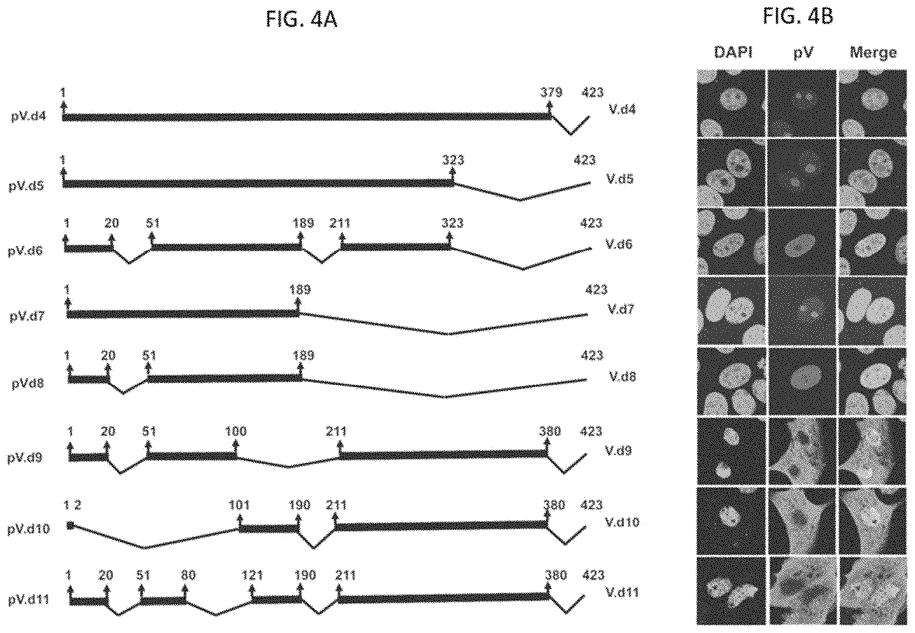

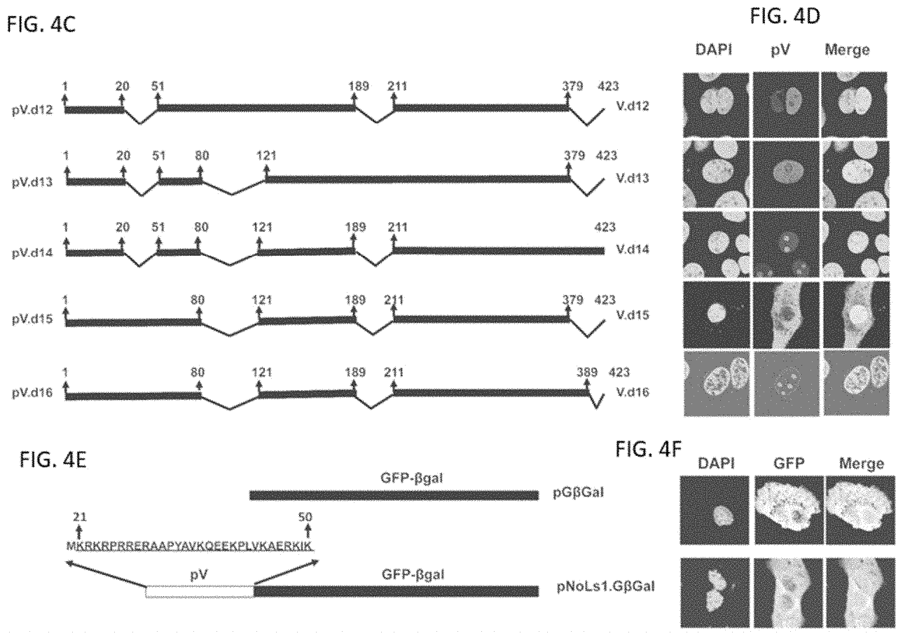

As demonstrated in the present examples described below, deletion analysis identified N-terminal amino acids 21-50 (NoLS1) and C-terminal amino acid 380-389 (NoLS2) as NoLS, both containing basic residues that can function as NoLS. Both NoLS1 or NoLS2 amino acids were sufficient to direct nucleolar import of a EYFP, a non-nucleolar protein. An earlier report, suggesting that NoLS are highly basic amino acids and are predominantly localized near N or C-terminus of the protein (Scott et al., 2010). Deletion of a potential NoLS did not reduce the nucleolar localization of pV. Like NoLS1 and NoLS2, three arginine and lysine rich motifs of NoLs1 appear to have redundant function as deletion of either NoLS or mutation of any arginine lysine rich motif of NoLs1 did not abrogate the nucleolar localization of BAV-3 pV. Interestingly, deletion of both abrogated the nucleolar localization of pV. However, deletion of potential NoLS did not alter the nuclear localization of pV. Moreover, V.d15 containing amino acid 21-50 (NoLS1) localized predominantly in the cytoplasm of the transfected cells and the fusion protein GFPβGal containing amino acid 21-50 showed no nuclear and nucleolar localization. These results suggest that the NoLS1 does not contain nuclear localization signal(s) required for pV to localize to the nucleus. In contrast, V.d16 containing amino acids 21-50 (NoLS1) and 380-389 (NoLS2) located in the nucleus and nucleolus (FIG. 4D), suggesting that amino acids 380-389 can mediate V.d16 both nuclear and nucleolar localization.

Nucleolar transport usually requires binding of nucleolar constituents to specific protein sequences, namely nucleolar localization signal (NoLS), which helps to retain the protein in the nucleolus. Though, there is no consensus of known NoLS sequences, NoLS are usually rich in lysine and arginine residues, which may interact with nucleolar RNAs or other nucleolar proteins (Olson and Dundr, 2005) for their retention in the nucleolus by a charge dependent mechanism (Musinova et al., 2015). While many nucleolar proteins contain RNA binding motifs (Hiscox, 2007) and are retained by binding to nucleolar RNAs, nucleophosmin protein contains acidic regions which bind to positively charged amino acids in putative nuclear proteins and retain them in the nucleolus (Adachi et al., 1993; Valdez et al., 1994). Although NoLS1 and NoLS2 do not contain a specific amino acid sequence, both are rich in positively charged basic residues. Since no specific NoLS sequence pattern could be defined in pV, the abundance of positively charged residues appears to mediate translocation of pV from nucleus to nucleolus suggesting that nucleolar retention is due to electrostatic interactions.

Unlike nucleolar transport, nuclear import requires active transport mechanisms, which are dependent on energy, soluble factors and functional nuclear pore complex (Nigg, 1997). Most of the proteins imported into the nucleus contain nuclear localization signals (Boulikas, 1993; Kosugi et al., 2009), which interact with importin α\fl and\or transportins in the cytoplasm and are transported through nuclear pore complex into the nucleus. Though bioinformatic analysis predicted 190-210 to act as potential NLS, deletion analysis identified three regions including amino acid 80-120, 190-210 and 380-423, which can act as NLS. Deletion of all three motifs is required to abolish the nuclear localization and binding of pV to importin α3 suggesting that each motif is functionally redundant. Separate or overlapping redundant NLSs have been identified in viral proteins including polyomavirus large T antigen (Howes et al., 1996; Richardson et al., 1986), influenza virus NS1 protein (Melen et al., 2007), adeno-associated virus 2 assembly activating protein (Earley et al., 2015) and in BAV-3 33K (Kulshreshtha et al., 2014). Without being bound by theory, it is possible that BAV-3 pV NLS redundancy may help promote efficient interaction with nuclear transport system leading to an effective nuclear transport. Support for this comes from the fact that increased binding of pV to importin α3 could be observed in the presence of all three NLS regions.

A number of viral proteins including HAdV-5 pVII use multiple nuclear import pathways (Wodrich et al., 2006). Recently, we also have demonstrated that nuclear import of BAV-3 33K involves recognition of overlapping NLS motifs located in 40 amino acid long conserved region of BAV-3 33K by importin α5 and transportin-3 (Kulshreshtha et al., 2014). Though transportin-3 has been shown to be required for HAdV-5 pV nucleolar transport, our data indicates that the nuclear import of BAV pV is mediated only by importin α3 of importin α/0 pathway and requires amino acids 81-120, 190-210, and 380-423.

Although deletion of NoLS2 affects the efficient production of progeny virus, both NoLS1 and NoLS2 do not appear essential for the production of viable virus suggesting that each NoLS motif may be functionally redundant. In contrast, deletion of both NoLS1 and NoLS2 prevented the production of viable virus suggesting that nucleolar localization of pV is essential for the production of viable virus. Since nucleolar delocalization of pV appeared lethal for production of progeny virus in MDBK cells, this phenotype could be due to defect in any step of the viral replication including viral protein expression, DNA replication and\or virus assembly. The early protein expression and genome replication in BAV.pVd1d3 appeared comparable to BAV304a suggesting that the loss of growth is potentially due to an event occurring late in infection. Analysis of late protein expression revealed that the nucleolar delocalization of pV altered the expression of some late viral proteins namely hexon, 100K and pV in BAV.pVd1d3 infected cells compared to BAV304a infected cells. Moreover, progeny virus could be detected in BAV.pVd1d3 infected MDBK cells suggesting that pV NoLSs are not required for assembly of empty capsids and immature virions. Western blot analysis of CsCl purified BAV.pVd1d3 virus grown in CRL cells could not detect difference in pV incorporation, indicating that pV NoLSs are not essential for pV incorporating into the virus particles.

Earlier reports have suggested that trimerization and nuclear transport of Hexon by 100K is required for formation of capsid (Hong et al., 2005; Xi et al., 2005). In the protein expression assay described in the present examples, the expression of both 100K and Hexon was decreased in NoLSs deleted BAV-3 infected cells. Thus, one explanation of the impaired viral assembly is the reduced expression of Hexon, as well as its decreased trimerization and nuclear transport because of the decreased expression of 100K. Moreover, in another study, the interactions of 33K with pV or 100K were detected (Kulshreshtha and Tikoo, 2008). We also found that pV can interact with 100K and 33K (Zhao and Tikoo, unpublished data). Therefore, one may speculate that pV may form a complex with 100K and 33K to manipulate not only 100K functions but also 33K functions. 33K has been proved to regulate the major late promoter (Ali et al., 2007), capsid assembly and capsid DNA interaction (Finnen et al., 2001; Kulshreshtha and Tikoo, 2008).

### A. Defective BAV Vectors

In some aspects, the invention provides defective bovine adenovirus (BAV) vectors comprising inverted terminal repeat sequences and BAV packaging sequences, wherein the BAV vector lacks pV functions. In some embodiment, the BAV vector comprises one or more modifications of the nucleic acid encoding pV wherein the pV lacks nuclear localization functions and/or nucleolar localization functions. Disruption of pV function may be accomplished by substitution, insertion or deletion of the pV region of the BAV genome. In some embodiments, the substitution, insertion or deletion may affect transcription, translation or post-translational modification of the pV. In some embodiments, the substitution, insertion, or deletion alters the function of a pV polypeptide produced from the pV region; for example, the substitution, insertion or deletion may alter nuclear localization and/or nucleolar localization of pV.

In some embodiment, the defective BAV vector comprises a deletion of part of or all of the coding region for pV. In some embodiments, the defective BAV vector comprises a deletion of all of the coding region for pV. In some embodiments, the defective BAV vector comprises a deletion corresponding to nucleotides 15068 to 16299 of SEQ ID NO:1. In some embodiments, the defective BAV vector comprises a deletion of nucleotides encoding amino acid residues 1-423 of the pV set forth in SEQ ID NO:2.

In some embodiments, the defective BAV vector comprises a deletion of part of the pV coding region. In some embodiments, the defective BAV vector comprises a deletion of part of the pV coding region that alters one or more functions of the pV. In some embodiments, the defective BAV vector comprises a deletion of part of the pV coding region that reduces or obliterates one or more functions of the pV. In some embodiments, the defective BAV vector comprises a deletion of part of the pV coding region that alters nuclear localization and/or nucleolar localization of the pV. In some embodiments, the defective BAV vector comprises a deletion of part of the pV coding region that reduces or obliterates nuclear localization and/or nucleolar localization of the pV. In some embodiments, the defective BAV vector comprises a deletion of nucleotides encoding amino acid residues 21-50 and 380-389 of the pV set forth in SEQ ID NO:2. In some embodiments, the defective BAV vector comprises a deletion of nucleotides encoding amino acid residues 21-50, 190-210 and 380-389 of the pV set forth in SEQ ID NO:2. In some embodiments, the defective BAV vector comprises a deletion of nucleotides encoding amino acid residues 21-50 and 380-423 of the pV set forth in SEQ ID NO:2. In some embodiments, the defective BAV vector comprises a deletion of nucleotides encoding amino acid residues 21-50, 190-210 and 380-423 of the pV set forth in SEQ ID NO:2. In some embodiments, the defective BAV vector comprises a deletion of nucleotides encoding amino acid residues 21-50, 190-210 and 323-423 of the pV set forth in SEQ ID NO:2. In some embodiments, the defective BAV vector comprises a deletion of nucleotides encoding amino acid residues 21-50, 190-423 of the pV set forth in SEQ ID NO:2. In some embodiments, the defective BAV vector comprises a deletion of nucleotides encoding amino acid residues 21-50, 101-210 and 380-423 of the pV set forth in SEQ ID NO:2. In some embodiments, the defective BAV vector comprises a deletion of nucleotides encoding amino acid residues 3-100, 190-210 and 380-423 of the pV set forth in SEQ ID NO:2. In some embodiments, the defective BAV vector comprises a deletion of nucleotides encoding amino acid residues 21-50, 81-120, 190-210 and 380-423 of the pV set forth in SEQ ID NO:2. In some embodiments, the defective BAV vector comprises a deletion of nucleotides encoding amino acid residues 81-120, 190-210 and 380-423 of the pV set forth in SEQ ID NO:2. In some embodiments, the defective BAV vector comprises a deletion of nucleotides encoding amino acid residues 81-120, 190-210 and 390-423 of the pV set forth in SEQ ID NO:2.

In some embodiments, the defective BAV vector comprises one or more amino acid substitutions in the pV coding region. In some embodiments, the defective BAV vector comprises one or more amino acid substitutions in the pV coding region that alters one or more functions of the pV. In some embodiments, the defective BAV vector comprises one or more amino acid substitutions in the pV coding region that reduces or obliterates one or more functions of the pV. In some embodiments, the defective BAV vector comprises one or more amino acid substitutions in the pV coding region that alters nuclear localization and/or nucleolar localization of the pV. In some embodiments, the defective BAV vector comprises one or more amino acid substitutions in the pV coding region that reduces or obliterates nuclear localization and/or nucleolar localization of the pV. In some embodiments, substitution of the nucleic acid encoding pV results in the substitution of one or more of amino acid residues 21-50 or 380-389 of the pV set forth in SEQ ID NO:2. In some embodiments, the pV comprises the sequence set forth in SEQ ID NO:15.

In some embodiments, the defective BAV vector further comprises a deletion of all or part of the E3 region.

In some embodiments the defective BAV vector further comprises nucleic acid encoding one or more heterologous transgenes. In some embodiments, the defective BAV vector comprised two, three, four, five or more than five heterologous transgenes. In some embodiments, the nucleic acid encoding the heterologous transgene is located in the E3 region. In some embodiments, the nucleic acid encoding the heterologous transgene is located in the pV region. In some embodiments, the nucleic acid encoding the heterologous transgene is located in the E3 region and in the pV region. In some embodiments, a first heterologous transgene is located in the E3 region and a second heterologous transgene is located in the pV region.

In some embodiments, the heterologous transgene encodes a therapeutic polypeptide or a therapeutic nucleic acid. In some embodiments, the heterologous transgene encodes a coagulation factor, a hormone, a cytokine, a lymphokine, an oncogene product, a tumor suppressor, a cell receptor, a ligand for a cell receptor, a protease inhibitor, an antibody, a toxin, an immunogenic polypeptide, an antibody, a dystrophin, a cystic fibrosis transmembrane conductance regulator (CFTR), siRNA, mRNA, miRNA, lncRNA, tRNA, or shRNA. In some embodiments, is any of the heterologous transgenes described herein.

In some embodiments, the nucleic acid encoding the transgene is operably linked to a promoter. Examples of promoters include, but are not limited to, the cytomegalovirus (CMV) immediate early promoter, the GUSB promoter, the RSV LTR, the MoMLV LTR, the phosphoglycerate kinase-1 (PGK) promoter, a simian virus 40 (SV40) promoter and a CK6 promoter, a transthyretin promoter (TTR), a TK promoter, a tetracycline responsive promoter (TRE), an HBV promoter, an hAAT promoter, a LSP promoter, chimeric liver-specific promoters (LSPs), the E2F promoter, the telomerase (hTERT) promoter; the cytomegalovirus enhancer/chicken beta-actin/Rabbit β-globin promoter (CAG promoter) and the elongation factor 1-alpha promoter (EF1-alpha) promoter.

In some embodiments, the BAV vector is a BAV vector of BAV serotype 1, 2, 3, 4, 5, 6, 7, 8, 9 or 10. In some embodiments, the BAV vector is a BAV vector of BAV serotype 1, 2, 3 or 10. In some embodiments, the rBAV particle is a BAV serotype 3 particle.

### B. Recombinant BAV Particles

In some aspects, the invention provides recombinant BAV (rBAV) particles wherein the rBAV particle comprises a defective rBAV genome comprising inverted terminal repeat sequences and BAV packaging sequences, wherein the rBAV genome lacks pV functions. In some embodiment, the rBAV particle comprises a rBAV genome comprising one or more modifications of the nucleic acid encoding pV wherein the pV lacks nuclear localization functions and/or nucleolar localization functions. Disruption of pV function may be accomplished by substitution, insertion or deletion of the pV region of the rBAV genome. In some embodiments, the substitution, insertion or deletion may affect transcription, translation or post-translational modification of the pV. In some embodiments, the substitution, insertion, or deletion alters the function of a pV polypeptide produced from the pV region; for example, the substitution, insertion or deletion may alter nuclear localization and/or nucleolar localization of pV.

In some embodiment, the rBAV particle comprises a defective rBAV genome comprising a deletion of part of or all of the coding region for pV. In some embodiments, the defective rBAV genome comprises a deletion of all of the coding region for pV. In some embodiments, the defective rBAV genome comprises a deletion corresponding to nucleotides 15068 to 16299 of SEQ ID NO:1. In some embodiments, the defective rBAV genome comprises a deletion of nucleotides encoding amino acid residues 1-423 of the pV set forth in SEQ ID NO:2.

In some embodiments, the rBAV particle comprises a defective rBAV genome comprising a deletion of part of the pV coding region. In some embodiments, the defective rBAV genome comprises a deletion of part of the pV coding region that alters one or more functions of the pV. In some embodiments, the defective rBAV genome comprises a deletion of part of the pV coding region that reduces or obliterates one or more functions of the pV. In some embodiments, the defective rBAV genome comprises a deletion of part of the pV coding region that alters nuclear localization and/or nucleolar localization of the pV. In some embodiments, the defective rBAV genome comprises a deletion of part of the pV coding region that reduces or obliterates nuclear localization and/or nucleolar localization of the pV. In some embodiments, the defective rBAV genome comprises a deletion of nucleotides encoding amino acid residues 21-50 and 380-389 of the pV set forth in SEQ ID NO:2. In some embodiments, the defective rBAV genome comprises a deletion of nucleotides encoding amino acid residues 21-50, 190-210 and 380-389 of the pV set forth in SEQ ID NO:2. In some embodiments, the defective rBAV genome comprises a deletion of nucleotides encoding amino acid residues 21-50 and 380-423 of the pV set forth in SEQ ID NO:2. In some embodiments, the defective rBAV genome comprises a deletion of nucleotides encoding amino acid residues 21-50, 190-210 and 380-423 of the pV set forth in SEQ ID NO:2. In some embodiments, the defective rBAV genome comprises a deletion of nucleotides encoding amino acid residues 21-50, 190-210 and 323-423 of the pV set forth in SEQ ID NO:2. In some embodiments, the defective rBAV genome comprises a deletion of nucleotides encoding amino acid residues 21-50, 190-423 of the pV set forth in SEQ ID NO:2. In some embodiments, the defective rBAV genome comprises a deletion of nucleotides encoding amino acid residues 21-50, 101-210 and 380-423 of the pV set forth in SEQ ID NO:2. In some embodiments, the defective rBAV genome comprises a deletion of nucleotides encoding amino acid residues 3-100, 190-210 and 380-423 of the pV set forth in SEQ ID NO:2. In some embodiments, the defective rBAV genome comprises a deletion of nucleotides encoding amino acid residues 21-50, 81-120, 190-210 and 380-423 of the pV set forth in SEQ ID NO:2. In some embodiments, the defective rBAV genome comprises a deletion of nucleotides encoding amino acid residues 81-120, 190-210 and 380-423 of the pV set forth in SEQ ID NO:2. In some embodiments, the defective rBAV genome comprises a deletion of nucleotides encoding amino acid residues 81-120, 190-210 and 390-423 of the pV set forth in SEQ ID NO:2.

In some embodiments, the rBAV particle comprises a defective rBAV genome comprising one or more amino acid substitutions in the pV coding region. In some embodiments, the defective rBAV genome comprises one or more amino acid substitutions in the pV coding region that alters one or more functions of the pV. In some embodiments, the defective rBAV genome comprises one or more amino acid substitutions in the pV coding region that reduces or obliterates one or more functions of the pV. In some embodiments, the defective BAV vector comprises one or more amino acid substitutions in the pV coding region that alters nuclear localization and/or nucleolar localization of the pV. In some embodiments, the defective rBAV genome comprises one or more amino acid substitutions in the pV coding region that reduces or obliterates nuclear localization and/or nucleolar localization of the pV. In some embodiments, substitution of the nucleic acid encoding pV results in the substitution of one or more of amino acid residues 21-50 or 380-389 of the pV set forth in SEQ ID NO:2. In some embodiments, the pV comprises the sequence set forth in SEQ ID NO:15.

In some embodiments, the defective rBAV genome of the rBAV particle further comprising a deletion of all or part of the E3 region.

In some embodiments the defective rBAV genome of the rBAV particle further comprises nucleic acid encoding one or more heterologous transgenes. In some embodiments, the defective rBAV genome comprised two, three, four, five or more than five heterologous transgenes. In some embodiments, the nucleic acid encoding the heterologous transgene is located in the E3 region. In some embodiments, the nucleic acid encoding the heterologous transgene is located in the pV region. In some embodiments, the nucleic acid encoding the heterologous transgene is located in the E3 region and in the pV region. In some embodiments, a first heterologous transgene is located in the E3 region and a second heterologous transgene is located in the pV region.

In some embodiments, the heterologous transgene encodes a therapeutic polypeptide or a therapeutic nucleic acid. In some embodiments, the heterologous transgene encodes a coagulation factor, a hormone, a cytokine, a lymphokine, an oncogene product, a tumor suppressor, a cell receptor, a ligand for a cell receptor, a protease inhibitor, an antibody, a toxin, an immunogenic polypeptide, an antibody, a dystrophin, a cystic fibrosis transmembrane conductance regulator (CFTR), siRNA, mRNA, miRNA, lncRNA, tRNA, or shRNA. In some embodiments, is any of the heterologous transgenes described herein.

In some embodiments, the nucleic acid encoding the transgene is operably linked to a promoter. Examples of promoters include, but are not limited to, the cytomegalovirus (CMV) immediate early promoter, the GUSB promoter, the RSV LTR, the MoMLV LTR, the phosphoglycerate kinase-1 (PGK) promoter, a simian virus 40 (SV40) promoter and a CK6 promoter, a transthyretin promoter (TTR), a TK promoter, a tetracycline responsive promoter (TRE), an HBV promoter, an hAAT promoter, a LSP promoter, chimeric liver-specific promoters (LSPs), the E2F promoter, the telomerase (hTERT) promoter; the cytomegalovirus enhancer/chicken beta-actin/Rabbit β-globin promoter (CAG promoter) and the elongation factor 1-alpha promoter (EF1-alpha) promoter.

In some embodiments, the rBAV particle is a BAV serotype 1, 2, 3, 4, 5, 6, 7, 8, 9 or 10 particle. In some embodiments, the rBAV particle is a BAV serotype 1, 2, 3 or 10 particle. In some embodiments, the rBAV particle is a BAV serotype 3 particle.

### C. Vaccines

In some aspects, the invention provides vaccines comprising a rBAV particle, wherein the rBAV particle comprises a defective rBAV genome comprising inverted terminal repeat sequences and BAV packaging sequences, wherein the rBAV genome lacks pV functions. In some embodiment, the rBAV particle comprises a rBAV genome comprising one or more modifications of the nucleic acid encoding pV wherein the pV lacks nuclear localization functions and/or nucleolar localization functions. Disruption of pV function may be accomplished by substitution, insertion or deletion of the pV region of the rBAV genome. In some embodiments, the substitution, insertion or deletion may affect transcription, translation or post-translational modification of the pV. In some embodiments, the substitution, insertion, or deletion alters the function of a pV polypeptide produced from the pV region; for example, the substitution, insertion or deletion may alter nuclear localization and/or nucleolar localization of pV.

In some embodiment, the vaccine comprises a rBAV particle with a defective rBAV genome comprising a deletion of part of or all of the coding region for pV. In some embodiments, the defective rBAV genome comprises a deletion of all of the coding region for pV. In some embodiments, the defective rBAV genome comprises a deletion corresponding to nucleotides 15068 to 16299 of SEQ ID NO:1. In some embodiments, the defective rBAV genome comprises a deletion of nucleotides encoding amino acid residues 1-423 of the pV set forth in SEQ ID NO:2.

In some embodiments, the vaccine comprises a rBAV particle with a defective rBAV genome comprising a deletion of part of the pV coding region. In some embodiments, the defective rBAV genome comprises a deletion of part of the pV coding region that alters one or more functions of the pV. In some embodiments, the defective rBAV genome comprises a deletion of part of the pV coding region that reduces or obliterates one or more functions of the pV. In some embodiments, the defective rBAV genome comprises a deletion of part of the pV coding region that alters nuclear localization and/or nucleolar localization of the pV. In some embodiments, the defective rBAV genome comprises a deletion of part of the pV coding region that reduces or obliterates nuclear localization and/or nucleolar localization of the pV. In some embodiments, the defective rBAV genome comprises a deletion of nucleotides encoding amino acid residues 21-50 and 380-389 of the pV set forth in SEQ ID NO:2. In some embodiments, the defective rBAV genome comprises a deletion of nucleotides encoding amino acid residues 21-50, 190-210 and 380-389 of the pV set forth in SEQ ID NO:2. In some embodiments, the defective rBAV genome comprises a deletion of nucleotides encoding amino acid residues 21-50 and 380-423 of the pV set forth in SEQ ID NO:2. In some embodiments, the defective rBAV genome comprises a deletion of nucleotides encoding amino acid residues 21-50, 190-210 and 380-423 of the pV set forth in SEQ ID NO:2. In some embodiments, the defective rBAV genome comprises a deletion of nucleotides encoding amino acid residues 21-50, 190-210 and 323-423 of the pV set forth in SEQ ID NO:2. In some embodiments, the defective rBAV genome comprises a deletion of nucleotides encoding amino acid residues 21-50, 190-423 of the pV set forth in SEQ ID NO:2. In some embodiments, the defective rBAV genome comprises a deletion of nucleotides encoding amino acid residues 21-50, 101-210 and 380-423 of the pV set forth in SEQ ID NO:2. In some embodiments, the defective rBAV genome comprises a deletion of nucleotides encoding amino acid residues 3-100, 190-210 and 380-423 of the pV set forth in SEQ ID NO:2. In some embodiments, the defective rBAV genome comprises a deletion of nucleotides encoding amino acid residues 21-50, 81-120, 190-210 and 380-423 of the pV set forth in SEQ ID NO:2. In some embodiments, the defective rBAV genome comprises a deletion of nucleotides encoding amino acid residues 81-120, 190-210 and 380-423 of the pV set forth in SEQ ID NO:2. In some embodiments, the defective rBAV genome comprises a deletion of nucleotides encoding amino acid residues 81-120, 190-210 and 390-423 of the pV set forth in SEQ ID NO:2.

In some embodiments, the vaccine comprises a rBAV particle with a defective rBAV genome comprising one or more amino acid substitutions in the pV coding region. In some embodiments, the defective rBAV genome comprises one or more amino acid substitutions in the pV coding region that alters one or more functions of the pV. In some embodiments, the defective rBAV genome comprises one or more amino acid substitutions in the pV coding region that reduces or obliterates one or more functions of the pV. In some embodiments, the defective BAV vector comprises one or more amino acid substitutions in the pV coding region that alters nuclear localization and/or nucleolar localization of the pV. In some embodiments, the defective rBAV genome comprises one or more amino acid substitutions in the pV coding region that reduces or obliterates nuclear localization and/or nucleolar localization of the pV. In some embodiments, substitution of the nucleic acid encoding pV results in the substitution of one or more of amino acid residues 21-50 or 380-389 of the pV set forth in SEQ ID NO:2. In some embodiments, the pV comprises the sequence set forth in SEQ ID NO:15.

In some embodiments, the vaccine comprises a rBAV particle with a defective rBAV genome, wherein the rBAV genome further comprises a deletion of all or part of the E3 region.

In some embodiments the defective rBAV genome of the vaccine further comprises nucleic acid encoding one or more heterologous transgenes. In some embodiments, the defective rBAV genome comprised two, three, four, five or more than five heterologous transgenes. In some embodiments, the nucleic acid encoding the heterologous transgene is located in the E3 region. In some embodiments, the nucleic acid encoding the heterologous transgene is located in the pV region. In some embodiments, the nucleic acid encoding the heterologous transgene is located in the E3 region and in the pV region. In some embodiments, a first heterologous transgene is located in the E3 region and a second heterologous transgene is located in the pV region.

In some embodiments, the heterologous transgene encodes a therapeutic polypeptide or a therapeutic nucleic acid. In some embodiments, the heterologous transgene encodes a coagulation factor, a hormone, a cytokine, a lymphokine, an oncogene product, a tumor suppressor, a cell receptor, a ligand for a cell receptor, a protease inhibitor, an antibody, a toxin, an immunogenic polypeptide, an antibody, a dystrophin, a cystic fibrosis transmembrane conductance regulator (CFTR), siRNA, mRNA, miRNA, lncRNA, tRNA, or shRNA. In some embodiments, is any of the heterologous transgenes described herein.

In some embodiments, the nucleic acid encoding the transgene is operably linked to a promoter. Examples of promoters include, but are not limited to, the cytomegalovirus (CMV) immediate early promoter, the GUSB promoter, the RSV LTR, the MoMLV LTR, the phosphoglycerate kinase-1 (PGK) promoter, a simian virus 40 (SV40) promoter and a CK6 promoter, a transthyretin promoter (TTR), a TK promoter, a tetracycline responsive promoter (TRE), an HBV promoter, an hAAT promoter, a LSP promoter, chimeric liver-specific promoters (LSPs), the E2F promoter, the telomerase (hTERT) promoter; the cytomegalovirus enhancer/chicken beta-actin/Rabbit β-globin promoter (CAG promoter) and the elongation factor 1-alpha promoter (EF1-alpha) promoter.

In some embodiments, the vaccine comprises a rBAV particle wherein the rBAV particle is a BAV serotype 1, 2, 3, 4, 5, 6, 7, 8, 9 or 10 particle. In some embodiments, the rBAV particle is a BAV serotype 1, 2, 3 or 10 particle. In some embodiments, the rBAV particle is a BAV serotype 3 particle.

### D. Pharmaceutical Compositions of BAV

The present invention also includes pharmaceutical compositions comprising a therapeutically effective amount of a defective BAV vector, recombinant BAV particle or vaccine as described herein. In some embodiments, the pharmaceutical composition comprises a defective BAV vector, recombinant BAV particle or vaccine as described in combination with a pharmaceutically acceptable excipient, vehicle and/or an adjuvant. In some embodiments, a defective BAV vector, recombinant BAV particle or vaccine, is prepared according to the methods of the invention in combination with a pharmaceutically acceptable excipient, vehicle and/or an adjuvant. Such a pharmaceutical composition can be prepared and dosages determined according to techniques that are well-known in the art. The pharmaceutical compositions of the invention can be administered by any known administration route including, but not limited to, systemically (for example, intravenously, intratracheally, intravascularly, intrapulmonarilly, intraperitoneally, intranasally, parenterally, enterically, intramuscularly, subcutaneously, intratumorally or intracranially) or by aerosolization or intrapulmonary instillation.

### IV. Host Cells

The invention provides host cells including any cell that will support production of the defective BAV vectors, rBAV particles or vaccines of the present invention. In some embodiments of the invention, recombinant cell lines are produced by constructing an expression cassette comprising the BAV pV region and transforming host cells therewith to provide complementing cell lines or cultures expressing the pV proteins. These recombinant complementing cell lines are capable of allowing a defective recombinant BAV lacking pV function to replicate. Complementing cell lines can provide pV functions through, for example, co-infection with a helper virus or by cointroduction of nucleic acid encoding the pV function. In other embodiments, complementing cell lines can provide pV functions by integrating or otherwise maintaining in stable form a fragment of a viral genome encoding a particular viral function (e.g., pV function).

In some embodiments, the invention provides a mammalian cell comprising nucleic acid encoding a BAV pV, said cell is capable of providing BAV pV function. In some embodiments, the BAV pV is BAV-3 pV. In some embodiments, the cell comprises nucleic acid encoding the BAV pV of SEQ ID NO:2. In some embodiments, the cell comprises nucleic acid encoding a BAV pV which has an amino acid sequence more than about 70%, 75%, 80%, 85%, 90%, 95%, or 99% identical to the amino acid sequence of SEQ ID NO:2. In some embodiments, the cell comprises nucleic acid of SEQ ID NO:X. In some embodiments, the cell comprises nucleic acid which has a nucleotide sequence more than about 40%, 50%, 60%, 70%, 80%, 90%, or 95% identical to the nucleotide sequence of SEQ ID NO:X.

In some embodiments, the nucleic acid encoding BAV pV is operably linked to a promoter. Examples of promoters include, but are not limited to, the cytomegalovirus (CMV) immediate early promoter, the GUSB promoter, the RSV LTR, the MoMLV LTR, the phosphoglycerate kinase-1 (PGK) promoter, a simian virus 40 (SV40) promoter and a CK6 promoter, a transthyretin promoter (TTR), a TK promoter, a tetracycline responsive promoter (TRE), an HBV promoter, an hAAT promoter, a LSP promoter, chimeric liver-specific promoters (LSPs), the E2F promoter, the telomerase (hTERT) promoter; the cytomegalovirus enhancer/chicken beta-actin/Rabbit β-globin promoter (CAG promoter; Niwa et al., Gene, 1991, 108(2):193-9) and the elongation factor 1-alpha promoter (EF1-alpha) promoter. In some embodiments, the nucleic acid encoding pV of the cell is operably linked to a CMV promoter.

In some embodiments, the host cell is a mammalian cell. In some embodiments, the host cell is a bovine cell. Exemplary cells include but are not limited to CRL cells or Madin-Darby bovine kidney (MDBK) cells. In some embodiments, the host cell is a CRL cell comprising nucleic acid encoding BAV pV. In some embodiments, the host cell is a CRL cell comprising nucleic acid encoding BAV pV having the amino acid sequence of SEQ ID NO:2. In some embodiments, the host cell is a CRL cell comprising nucleic acid encoding BAV pV having the amino acid sequence of SEQ ID NO:2 wherein the nucleic acid encoding the BAV pV is stably integrated into the host cell chromosome. In some embodiments, the host cell is a CRL cell comprising nucleic acid encoding BAV pV under the control of a CMV promoter. In some embodiments, the host cell is a CRL cell comprising nucleic acid encoding BAV pV having the amino acid sequence of SEQ ID NO:2 under the control of a CMV promoter. In some embodiments, the host cell is a CRL cell comprising nucleic acid encoding BAV pV having the amino acid sequence of SEQ ID NO:2 under the control of a CMV promoter wherein the nucleic acid encoding the BAV pV is stably integrated into the host cell chromosome.

### IV. Methods to Produce BAV Vectors

Methods to produce recombinant BAV vectors and to generate recombinant BAV particles are known in the art. For example, see WO 95/16048, WO 98/59063, WO 00/26395, WO 01/92547. Suitable host cells include any cell that will support recombination between a BAV genome and a plasmid containing BAV sequences, or between two or more plasmids, each containing BAV sequences. Recombination is generally performed in prokaryotic cells, such as E. coli, while transfection of a plasmid containing a viral genome, to generate virus particles, is conducted in eukaryotic cells, for example mammalian cells. In some embodiments, the mammalian cells are bovine cell cultures; for example MDBK or PFBR cells, and their equivalents. The growth of bacterial cell cultures, as well as culture and maintenance of eukaryotic cells and mammalian cell lines are procedures which are well-known to those of skill in the art.

Deletion of BAV pV can be accomplished by methods well-known to those of skill in the art. For example, for BAV sequences cloned in a plasmid, digestion with one or more restriction enzymes (with at least one recognition sequence in the BAV insert) followed by ligation will, in some cases, result in deletion of sequences between the restriction enzyme recognition sites. In some embodiments, nucleic acid amplification may be used to amplify specific regions of the BAV genome encompassing the desired region to be deleted, followed by restriction or other means to remove the sequences to be deleted.

One or more heterologous transgene sequences can be inserted into one or more regions of the BAV genome to generate a recombinant BAV, limited only by the insertion capacity of the BAV genome and ability of the recombinant BAV to express the inserted heterologous transgene sequences. In general, adenovirus genomes can accept inserts of approximately 5% of genome length and remain capable of being packaged into virus particles. The insertion capacity can be increased by deletion of non-essential regions and/or deletion of essential regions, such as, for example, pV function, whose function is provided by a helper cell line, such as one providing pV function. In some embodiments, a heterologous polynucleotide encoding a protein is inserted into an adenovirus E3 gene region. In other embodiments, an adenovirus has a deletion of part or all of the E3 region.

In one embodiment of the invention, insertion can be achieved by constructing a plasmid containing the region of the BAV genome into which insertion is desired, such as the E3 region. The plasmid is then digested with a restriction enzyme having a recognition sequence in the BAV portion of the plasmid, and a heterologous transgene sequence is inserted at the site of restriction digestion. The plasmid, containing a portion of the BAV genome with an inserted heterologous transgene sequence, is co-transformed, along with a BAV genome or a linearized plasmid containing a BAV genome, into a bacterial cell (such as, for example, E. coli), wherein the BAV genome can be a full-length genome or can contain one or more deletions. Homologous recombination between the plasmids generates a recombinant BAV genome containing inserted heterologous transgene sequences.

Deletion of BAV sequences, to provide a site for insertion of heterologous transgene sequences or to provide additional capacity for insertion at a different site, can be accomplished by methods well-known to those of skill in the art. For example, for BAV sequences cloned in a plasmid, digestion with one or more restriction enzymes (with at least one recognition sequence in the BAV insert) followed by ligation will, in some cases, result in deletion of sequences between the restriction enzyme recognition sites. Alternatively, digestion at a single restriction enzyme recognition site within the BAV insert, followed by exonuclease treatment, followed by ligation will result in deletion of BAV sequences adjacent to the restriction site. A plasmid containing one or more portions of the BAV genome with one or more deletions, constructed as described above, can be co-transfected into a bacterial cell along with a BAV genome (full-length or deleted) or a plasmid containing either a full-length or a deleted BAV genome to generate, by homologous recombination, a plasmid containing a recombinant BAV genome with a deletion at one or more specific sites. BAV virions containing the deletion can then be obtained by transfection of mammalian cells (including, but not limited to, MDBK or PFBR cells and their equivalents) with the plasmid containing the recombinant BAV genome.

In one embodiment of the invention, insertion sites are adjacent to and downstream (in the transcriptional sense) of BAV promoters. Locations of BAV promoters, and restriction enzyme recognition sequences downstream of BAV promoters, for use as insertion sites, can be easily determined by one of skill in the art from the BAV nucleotide sequence provided herein. Alternatively, various in vitro techniques can be used for insertion of a restriction enzyme recognition sequence at a particular site, or for insertion of heterologous transgene sequences at a site that does not contain a restriction enzyme recognition sequence. Such methods include, but are not limited to, oligonucleotide-mediated heteroduplex formation for insertion of one or more restriction enzyme recognition sequences (see, for example, Zoller et al. (1982) Nucleic Acids Res. 10:6487-6500; Brennan et al. (1990) Roux's Arch. Dev. Biol. 199:89-96; and Kunkel et al. (1987) Meth. Enzymology 154:367-382) and PCR-mediated methods for insertion of longer sequences. See, for example, Zheng et al. (1994) Virus Research 31: 163-186.

It is also possible to obtain expression of a heterologous transgene sequence inserted at a site that is not downstream from a BAV promoter, if the heterologous transgene sequence additionally comprises transcriptional regulatory sequences that are active in eukaryotic cells. Such transcriptional regulatory sequences can include cellular promoters such as, for example, the bovine hsρ70 promoter and viral promoters such as, for example, herpesvirus, adenovirus and papovavirus promoters and DNA copies of retroviral long terminal repeat (LTR) sequences.

In another embodiment, homologous recombination in a prokaryotic cell can be used to generate a cloned BAV genome; and the cloned BAV genome can be propagated as a plasmid. See for example, U.S. Pat. No. 5,922,576. Infectious virus can be obtained by transfection of mammalian cells with the cloned BAV genome rescued from plasmid-containing cells.

The invention also provides BAV regulatory sequences which can be used to regulate the expression of heterologous genes. A regulatory sequence can be, for example, a transcriptional regulatory sequence, a promoter, an enhancer, an upstream regulatory domain, a splicing signal, a polyadenylation signal, a transcriptional termination sequence, a translational regulatory sequence, a ribosome binding site and a translational termination sequence. In another embodiment, the cloned BAV genome can be propagated as a plasmid and infectious virus can be rescued from plasmid-containing cells.

The presence of viral nucleic acids can be detected by techniques known to one of skill in the art including, but not limited to, hybridization assays, polymerase chain reaction, and other types of amplification reactions. Similarly, methods for detection of proteins are well-known to those of skill in the art and include, but are not limited to, various types of immunoassay, ELISA, Western blotting, enzymatic assay, immunohistochemistry, etc. Diagnostic kits comprising the nucleotide sequences of the invention may also contain reagents for cell disruption and nucleic acid purification, as well as buffers and solvents for the formation, selection and detection of hybrids. Diagnostic kits comprising the polypeptides or amino acid sequences of the invention may also comprise reagents for protein isolation and for the formation, isolation, purification and/or detection of immune complexes.

Various foreign genes or nucleotide sequences or coding sequences (prokaryotic, and eukaryotic) can be inserted in the bovine adenovirus nucleotide sequence, e.g., DNA, in accordance with the present invention, particularly to provide protection against a wide range of diseases and many such genes are already known in the art. The problem heretofore has been to provide a safe, convenient and effective vaccine vector for the genes or sequences, as well as safe, effective means for gene transfer to be used in various gene therapy applications.

A heterologous nucleotide sequence can consist of one or more gene(s) of interest, and preferably of therapeutic interest. In the context of the present invention, a heterologous transgene of interest can code either for a regulatory RNA (e.g., siRNA, miRNA, lncRNA, tRNA, or shRNA), a ribozyme or for an mRNA which will then be translated into a protein of interest. A transgene of interest may be of genomic type, of complementary DNA (cDNA) type or of mixed type (minigene, in which at least one intron is deleted). It can code for a mature protein, a precursor of a mature protein, in particular a precursor intended to he secreted and accordingly comprising a signal peptide, a chimeric protein originating from the fusion of sequences of diverse origins, or a mutant of a natural protein displaying improved or modified biological properties. Such a mutant may be obtained by, deletion, substitution and/or addition of one or more nucleotide(s) of the gene coding for the natural protein, or any other type of change in the sequence encoding the natural protein, such as, for example, transposition or inversion.

A heterologous transgene of interest may be placed under the control of elements (DNA control sequences) suitable for its expression in a host cell. Suitable DNA control sequences are understood to mean the set of elements needed for transcription of a gene into RNA (regulatory RNA, mRNA, etc.) and in some examples, for the translation of an mRNA into protein. In some embodiments, the promoter can be a constitutive promoter or a regulatable promoter, and can be isolated from any gene of eukaryotic, prokaryotic or viral origin, and even adenoviral origin. Alternatively, it can be the natural promoter of the gene of interest. Generally speaking, a promoter used in the present invention may be modified so as to contain regulatory sequences. As examples, a gene of interest in use in the present invention is placed under the control of the promoter of the immunoglobulin genes when it is desired to target its transfer to lymphocytic host cells. There may also be mentioned the HSV-1 TK (herpesvirus type 1 thymidine kinase) gene promoter, the adenoviral MLP (major late promoter), in particular of human adenovirus type 2, the RSV (Rous Sarcoma Virus) LTR (long terminal repeat), the CMV (Cytomegalovirus) early promoter, and the PGK (phosphoglycerate kinase) gene promoter, for example, permitting expression in a large number of cell types.

Heterologous transgenes of interest for use in the defective BAV vectors and of the present invention include but are not limited to the following: genes coding for cytokines such as interferons and interleukins; genes encoding lymphokines; genes coding for membrane receptors such as the receptors recognized by pathogenic organisms (viruses, bacteria or parasites), for example, receptors recognized by the HIV virus (human immunodeficiency virus); genes coding for coagulation factors such as factor VIII and factor IX; genes coding for dystrophins; genes coding for insulin; genes coding for proteins participating directly or indirectly in cellular ion channels, such as the CFTR (cystic fibrosis transmembrane conductance regulator) protein; genes coding for regulatory RNAs (e.g., siRNA, miRNA, lncRNA, tRNA, or shRNA), or proteins capable of inhibiting the activity of a protein produced by a pathogenic gene which is present in the genome of a pathogenic organism, or proteins (or genes encoding them) capable of inhibiting the activity of a cellular gene whose expression is deregulated, for example an oncogene; genes coding for a protein inhibiting an enzyme activity, such as a-antitrypsin or a viral protease inhibitor, for example; genes coding for variants of pathogenic proteins which have been mutated so as to impair their biological function, such as, for example, trans-dominant variants of the tat protein of the HIV virus which are capable of competing with the natural protein for binding to the target sequence, thereby preventing the activation of HIV; genes coding for antigenic epitopes in order to increase the host cell's immunity; genes coding for major histocompatibility complex classes I and II proteins, as well as the genes coding for the proteins which are inducers of these genes; genes coding for antibodies; immunosuppressant genes; immunostimulatory genes; genes encoding nucleic acid and enzymes for gene editing; genes coding for immunotoxins; genes encoding toxins; genes encoding growth factors or growth hormones; genes encoding cell receptors and their ligands; genes encoding tumor suppressors; genes involved in cardiovascular disease including, but not limited to, oncogenes; genes encoding growth factors including, but not limited to, fibroblast growth factor (FGF), vascular endothelial growth factor (VEGF), and nerve growth factor (NGF); e-nos, tumor suppressor genes including, but not limited to, the Rb (retinoblastoma) gene; lipoprotein lipase; superoxide dismutase (SOD); catalase; oxygen and free radical scavengers; apolipoproteins; and pai-1 (plasminogen activator inhibitor-1); genes coding for cellular enzymes or those produced by pathogenic organisms; and suicide genes. The HSV-1 TK suicide gene may be mentioned as an example (this viral TK enzyme displays markedly greater affinity compared to the cellular TK enzyme for certain nucleoside analogues (such as acyclovir or gancyclovir). It converts them to monophosphorylated molecules, which can themselves be converted by cellular enzymes to nucleotide precursors, which are toxic. These nucleotide analogues can be incorporated into replicating DNA molecules, hence incorporation occurs chiefly in the DNA of dividing cells. This incorporation can result in specific destruction of dividing cells such as cancer cells.). This list is not restrictive, and other genes of interest may be used in the context of the present invention. In some embodiments, only fragments of nucleic acid sequences of genes can be used (where these are sufficient to generate a protective immune response or a specific biological effect) rather than the complete sequence as found in the wild-type organism.

In some embodiments, synthetic genes or fragments thereof can also be used.

However, the present invention can be used with a wide variety of genes, fragments and the like, and is not limited to those set out above.

In some cases the gene for a particular antigen can contain a large number of introns or can be from an RNA virus, in these cases a complementary DNA copy (cDNA) can be used.

In order for successful expression of the gene to occur, it can be inserted into an expression vector together with a suitable promoter including enhancer elements and polyadenylation sequences. A number of eukaryotic promoter and polyadenylation sequences which provide successful expression of foreign genes in mammalian cells and construction of expression cassettes, are known in the art, for example in U.S. Pat. No. 5,151,267, the disclosures of which are incorporated herein by reference. The promoter is selected to give optimal expression of immunogenic protein which in turn satisfactorily leads to humoral, cell mediated and mucosal immune responses according to known criteria.

The foreign protein produced by expression in vivo in a recombinant virus-infected cell may be itself immunogenic. More than one foreign gene can be inserted into the viral genome to obtain successful production of more than one effective protein.

Thus with the recombinant viruses of the present invention, it is possible to provide protection against a wide variety of diseases affecting cattle, humans and other mammals.

Any of the recombinant antigenic determinants or recombinant live viruses of the invention can be formulated and used in substantially the same manner as described for antigenic determinant vaccines or live vaccine vectors.

The present invention also includes pharmaceutical compositions comprising a therapeutically effective amount of a recombinant adenovirus vector, recombinant adenovirus or recombinant protein, prepared according to the methods of the invention, in combination with a pharmaceutically acceptable vehicle and/or an adjuvant. Such a pharmaceutical composition can be prepared and dosages determined according to techniques that are well-known in the art. The pharmaceutical compositions of the invention can be administered by any known administration route including, but not limited to, systemically (for example, intravenously, intratracheally, intravascularly, intrapulmonarilly, intraperitoneally, intranasally, parenterally, enterically, intramuscularly, subcutaneously, intratumorally or intracranially) or by aerosolization or intrapulmonary instillation. Administration can take place in a single dose or in doses repeated one or more times after certain time intervals. The appropriate administration route and dosage will vary in accordance with the situation (for example, the individual being treated, the disorder to be treated or the gene or polypeptide of interest), but can be determined by one of skill in the art.

In some embodiments, the invention provides a method of treatment, according to which a therapeutically effective amount of a BAV vector, recombinant BAV, or host cell of the invention is administered to a mammalian subject requiring treatment.

The antigens used in the present invention can be either native or recombinant antigenic polypeptides or fragments. They can be partial sequences, full-length sequences, or even fusions (e.g., having appropriate leader sequences for the recombinant host, or with an additional antigen sequence for another pathogen). The preferred antigenic polypeptide to be expressed by the virus systems of the present invention contain full-length (or near full-length) sequences encoding antigens. Alternatively, shorter sequences that are antigenic (i.e., encode one or more epitopes) can be used. The shorter sequence can encode a “neutralizing epitope,” which is defined as an epitope capable of eliciting antibodies that neutralize virus infectivity in an in vitro assay. In some embodiments, the peptide encodes a “protective epitope” that is capable of raising in the host a “protective immune response;” i.e., an antibody- and/or a cell-mediated immune response that protects an immunized host from infection.

In some embodiments, the antigens used in the present invention (e.g., when comprised of short oligopeptides) can be conjugated to a vaccine carrier. Vaccine carriers are well known in the art: for example, bovine serum albumin (BSA), human serum albumin (HSA) and keyhole limpet hemocyanin (KLH). A preferred carrier protein, rotavirus VP6, is disclosed in EPO Pub. No. 0259149, the disclosure of which is incorporated by reference herein.

In some embodiments, genes for desired antigens or coding sequences thereof which can be inserted include those of organisms which cause disease in mammals, particularly bovine pathogens such as bovine rotavirus, bovine coronavirus, bovine herpes virus type 1, bovine respiratory syncytial virus, bovine parainfluenza virus type 3 (BPI-3), bovine diarrhea virus, Pasteurella haemolytica, Haemophilus somnus and the like. Genes encoding antigens of human pathogens also useful in the practice of the invention. The vaccines of the invention carrying foreign genes or fragments can also be orally administered in a suitable oral carrier, such as in an enteric-coated dosage form. Oral formulations include such normally-employed excipients as, for example, pharmaceutical grades of mannitol, lactose, starch, magnesium stearate, sodium saccharin cellulose, magnesium carbonate, and the like. Oral vaccine compositions may be taken in the form of solutions, suspensions, tablets, pills, capsules, sustained release formulations, or powders, containing from about 10% to about 95% of the active ingredient, preferably about 25% to about 70%. Oral and/or intranasal vaccination may be preferable to raise mucosal immunity (which plays an important role in protection against pathogens infecting the respiratory and gastrointestinal tracts) in combination with systemic immunity.

In addition, the vaccine can be formulated into a suppository. For suppositories, the vaccine composition will include traditional binders and carriers, such as polyalkaline glycols or triglycerides. Such suppositories may be formed from mixtures containing the active ingredient in the range of about 0.5% to about 10% (w/w), preferably about 1% to about 2%.

Protocols for administering to animals the vaccine composition(s) of the present invention are within the skill of the art in view of the present disclosure. Those skilled in the art will select a concentration of the vaccine composition in a dose effective to elicit an antibody and/or T-cell mediated immune response to the antigenic fragment. Within wide limits, the dosage is not believed to be critical. Typically, the vaccine composition is administered in a manner which will deliver between about 1 to about 1,000 micrograms of the subunit antigen in a convenient volume of vehicle, e.g., about 1-10 cc. Preferably, the dosage in a single immunization will deliver from about 1 to about 500 micrograms of subunit antigen, more preferably about 5-10 to about 100-200 micrograms (e.g., 5-200 micrograms).

The timing of administration may also be important. For example, a primary inoculation preferably may be followed by subsequent booster inoculations if needed. It may also be preferred, although optional, to administer a second, booster immunization to the animal several weeks to several months after the initial immunization. To insure sustained high levels of protection against disease, it may be helpful to readminister a booster immunization to the animals at regular intervals, for example once every several years. Alternatively, an initial dose may be administered orally followed by later inoculations, or vice versa. Preferred vaccination protocols can be established through routine vaccination protocol experiments.

The dosage for all routes of administration of in vivo recombinant virus vaccine depends on various factors including, the size of patient, nature of infection against which protection is needed, carrier and the like and can readily be determined by those of skill in the art. By way of non-limiting example, a dosage of between 10³ pfu and 10¹⁵ pfu, between 10⁵ and 10¹³ pfu, or between 10⁶ to 10¹¹ pfu and the like can be used. As with in vitro subunit vaccines, additional dosages can be given as determined by the clinical factors involved.

The invention also provides methods for providing gene delivery to a mammal, such as a bovine or a human or other mammal in need thereof, to control a gene deficiency, to provide a therapeutic gene or nucleotide sequence and/or to induce or correct a gene mutation. The method can be used, for example, in the treatment of conditions including, but not limited to hereditary disease, infectious disease, cardiovascular disease, and viral infection. The method comprises administering to said mammal a live recombinant bovine adenovirus comprising a modification in a capsid protein, or fragment thereof, wherein said capsid protein is associated with tropism and said modification is associated with altered tropism and wherein said adenovirus vector further comprises a foreign polynucleotide sequence encoding a non-defective form of said gene under conditions wherein the recombinant virus vector genome is incorporated into said mammalian genome or is maintained independently and extrachromosomally to provide expression of the required gene in the target organ or tissue. These kinds of techniques are currently being used by those of skill in the art for the treatment of a variety of disease conditions, non-limiting examples of which are provided above. Examples of foreign genes, nucleotide sequences or portions thereof that can be incorporated for use in a conventional gene therapy include, cystic fibrosis transmembrane conductance regulator gene, human minidystrophin gene, alpha-1-antitrypsin gene, genes involved in cardiovascular disease, and the like.

In some embodiments, the practice of the present invention in regard to gene delivery in humans is intended for the prevention or treatment of diseases including, but not limited to, genetic diseases (for example, hemophilia, thalassemias, emphysema, Gaucher's disease, cystic fibrosis, Duchenne muscular dystrophy, Duchenne's or Becker's myopathy, etc.), cancers, viral diseases (for example, AIDS, herpesvirus infection, cytomegalovirus infection and papillomavirus infection), cardiovascular diseases, and the like. For the purposes of the present invention, the vectors, cells and viral particles prepared by the methods of the invention may be introduced into a subject either ex vivo, (i.e., in a cell or cells removed from the patient) or directly in vivo into the body to be treated.

Compositions of the invention (e.g., defective BAV vectors) can be used either alone or in combination with one or more additional therapeutic agents for treating any or all of the disorders described herein. The interval between sequential administration can be in terms of at least (or, alternatively, less than) minutes, hours, or days.

### VI. Articles of Manufacture and Kits

The compositions as described herein (e.g., comprising a defective BAV vector) may be contained in an article of manufacture or kit, e.g., within a system, designed for use in one of the methods of the invention as described herein. The kits may comprise any of the nucleic acids, chimeric introns, 5′UTRs, expression constructs, vectors, defective BAV vectors, cells, viral particles, rABAV particles, and/or pharmaceutical compositions of the invention.

In some embodiments, the kits further contain buffers and/or pharmaceutically acceptable excipients. As is well known in the art, pharmaceutically acceptable excipients are relatively inert substances that facilitate administration of a pharmacologically effective substance and can be supplied as liquid solutions or suspensions, as emulsions, or as solid forms suitable for dissolution or suspension in liquid prior to use. For example, an excipient can give form or consistency, or act as a diluent. Suitable excipients include but are not limited to stabilizing agents, wetting and emulsifying agents, salts for varying osmolarity, encapsulating agents, pH buffering substances, and buffers. In some embodiments, such excipients include any pharmaceutical agent suitable for direct delivery to the eye which may be administered without undue toxicity. Pharmaceutically acceptable excipients include, but are not limited to, sorbitol, any of the various TWEEN compounds, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in REMINGTON'S PHARMACEUTICAL SCIENCES (Mack Pub. Co., N. J. 1991).

In some embodiments, pharmaceutically acceptable excipients may include pharmaceutically acceptable carriers. Such pharmaceutically acceptable carriers can be sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. Additional ingredients may also be used, for example preservatives, buffers, tonicity agents, antioxidants and stabilizers, nonionic wetting or clarifying agents, viscosity-increasing agents, and the like. The kits described herein can be packaged in single unit dosages or in multidosage forms. The contents of the kits are generally formulated as sterile and substantially isotonic solution.

In some embodiments, the kits further include instructions for delivery of the composition (e.g., of defective BAV vectrs or viral particles). The kits described herein may further include other materials desirable from a commercial and user standpoint, including other buffers, diluents, filters, needles, syringes, and package inserts with instructions for performing any methods described herein. Suitable packaging materials may also be included and may be any packaging materials known in the art, including, for example, vials (such as sealed vials), vessels, ampules, bottles, jars, flexible packaging (e.g., sealed Mylar or plastic bags), and the like. These articles of manufacture may further be sterilized and/or sealed. In some embodiments, the kits comprise instructions for treating a disorder described herein using any of the methods and/or compositions described herein.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Materials and Method

The following materials and methods were used for the Examples unless indicated otherwise.

### Cells and Viruses

Madin Darby bovine kidney (MDBK), CRL (Cotton rat lung) cells (Papp et al., 1997), VIDO-DT1 (cotton rat lung (CRL) cells expressing I-SceI) and CRL.pV cells (described below) were cultivated in minimal essential medium (MEM) (Sigma) supplemented with 10% heat-inactivated fetal bovine serum (FBS, Invitrogen). The HEK293T cells (ATCC® CRL-3216TM) were cultivated in Dulbecco's minimal essential medium (DMEM) (Sigma) with 10% FBS. BAV304a (BadV-3 E3 region was replaced by a EYFP gene) was propagated in MDBK cells, and mutant BadV-3s were propagated in MDBK or CRL.pV cells.

### Antibodies

To produce BAdV-3 pV specific sera, two peptides representing amino acid 1-24 (XZ1) and amino acids 180-212 (XZ2) were synthesized by Genscript. Rabbits were immunized with individual (500 μg/rabbit) peptide conjugated to key hole limpet haemocyanin emulsified with Freund's complete adjuvant followed by two injections of ovalbumin conjugated individual peptide (300 μg/rabbit) in Freund's incomplete adjuvant three weeks apart. Sera were collected ten days after third injection and tested for specificity by Western blotting. Commercial antibodies used include RPA194 Antibody (C-1) (Santa Cruz Biotechnology), Anti-β-actin monoclonal antibody (Sigma-Aldrich), Alexa Fluor 488-conjugated goat anti-rabbit IgG (Jackson Immunoresearch), TRITC-conjugated goat anti-mouse IgG (Jackson Immunoresearch), TRITC-conjugated goat anti-rabbit IgG (Jackson Immunoresearch), Alexa Fluor 647-conjugated goat anti-rabbit IgG (Invitrogen), Alexa Fluor 680 conjugated goat anti-rabbit antibody (Invitrogen), and IRDye800 conjugated goat anti-mouse antibody (Rockland).

### Construction of pV Expressing Cell Line CRL.pV

Earlier, a lentivirus system was successfully used to isolate VIDO DT1 cells expressing I-SceI endonuclease) (Du and Tikoo, 2010). To isolate the cell line stably expressing BAdV-3 pV, a second generation replication defective lentivirus system was used containing cloning plasmid pTrip-puro, plasmid pSPAX expressing HIV Gag/Pol proteins and plasmid pMD2. G, expressing vesicular stomatitis virus G protein. Briefly, a 1.2 kb DNA fragment containing BAdV-3 pV gene was ligated to EcoRV-XhoI digested plasmid pTrip-puro (containing a puromycin resistant marker), creating plasmid pTrip-pV-Puro. The HEK293T cells were co-transfected with plasmid (pTrip-pV-Puro, pSPAX2 and pSPAX) DNAs. At 48 h post-transfection, the lentivirus in media was collected and used to transduce CRL cells with 8 μg/μl polybrene. At 24 h post-transduction, the transduced cells were transferred to 10 cm2 dishes. After 24 h, media were replaced by fresh selection media containing 5 μg/ml of puromycin. The puromycin resistant cell clones were picked, propagated in puromycin containing media and tested for the expression of BAdV-3 pV.

### Western Blot Analysis

Proteins from purified virus, virus infected cell lysates or pV expressing cell lysates were separated by Sodium dodecyl-sulfate (SDS) polyacrylamide gel electrophoresis (PAGE), transferred to nitrocellulose membrane (Bio-Rad) and probed by Western blot using protein specific anti-serum and Alexa Fluor 680, alkaline phosphatase (AP)-conjugated goat anti-rabbit IgG (Sigma), or IRDye800 conjugated antibodies. The membranes probed with fluorophore-conjugated secondary antibody were scanned and analyzed by Odyssey® CLx Imaging System (LI-COR).

### Confocal Microscopy

Cells in 2-well chamber slides were fixed with 3.7% paraformaldehyde for 15 min and permeabilized with 0.1 M PBS containing 0.5% Triton X-100. After blocking with 5% goat serum, the cells were stained with rabbit anti-serum and fluorophor conjugated goat anti-rabbit IgG (Jackson Immunoresearch). Finally, the cells were mounted by mounting buffer (Vector Laboratories Inc.) containing DAPI and imagined under confocal microscope TCS SP5 (Leica).

### GST-Pull Down Assay

GST fusion proteins (Importin α1, α3, α5, α7, β1 or transportin-1) were purified from plasmids transfected E. coli BL21 by glutathione sepharose beads (GE Healthcare). [³⁵S] methionine-labeled pV was synthesized and labeled by TNT T7 Quick Coupled Transcription/Translation System (Invitrogen). After incubation of GST fusion proteins and [35S] methionine-labeled pV overnight, the samples were separated by 10% SDS-PAGE. After exposed the gel overnight, the phosphor screen was scanned by Molecular Imager FX (Bio-Rad).

### Isolation of Mutant BAdV-3s

VIDO DT1 (CRL cells expressing I-SceI recombinase) cells or CRL. PV(CRL cells expressing BAdV-3 pV) cells in six-well plate were transfected with individual plasmid DNA with Lipofectamine 2000 (Invitrogen). At 4 hrs posttransfection, the media were replaced with fresh MEM containing 2% FBS. Transfected cells showing cytopathic effect (CPE) were harvested, freeze-thawed three times. The recombinant virus was propagated in MDBK or CRL.pV cells.

### Virus DNA Replication

The CRL cells were infected with BAV304a or BAV.dV at a MOT of 2. At 12, 24 and 36 hrs post infection, the infected cells were washed in phosphate buffered saline and used to purify low molecular weight DNA as described (Farina et al., 2001). Equal amount of DNA was digested with Bmt1 restriction enzyme and separated by agarose gel electrophoresis and analyzed by Gel DocTM XR+System (Bio-Rad).

### CsCl Gradient Centrifugation

Monolayers of MDBK or CRL.pV (CRL cells expressing BAdV-3 pV) cells in T-150 Flasks were infected with wild-type or mutant BAdV-3s at a multiplicity of infection of 5. At 48 h post-infection, the cells were collected and resuspended in 5 ml medium. After three times freeze-thawing, the cell lysates were subjected to CsCl density gradient centrifugation at 35 000 rpm for 1 hr at 4° C. The bands containing viruses were collected, and subjected to a second centrifugation at 35 000 rpm for overnight at 4° C. At last, the virus band was collected, dialyzed three times to remove the trace amount of cesium chloride and stored in small aliquots at −80° C.

### Virus Single Cycle Growth Curve

MDBK or CRL cells in 24-well plates were infected with wild-type or mutant BAdV-3s at a multiplicity of infection of 1 or 2. At indicated time points post infection, the infected cells were harvested, lysed by freeze-thawing three times to release the virus into medium, and then used to determine virus titer by TCID₅₀ in CRL.pV cells (CRL cells expressing BAdV-3 pV) as described elsewhere (Kulshreshtha et al., 2004).

### Virus Thermostability Assay

To determine the thermostability of BAV304a and mutant BAdV-3s, 10⁵ purified infectious viral particles were incubated at different temperatures (−80° C., −20° C., 4° C., 25° C. and 37° C.) for three days in PBS containing 10% glycerol. To assess the different dynamics of viral inactivation, 10⁵ infectious purified viral particles were incubated at different temperatures (−80° C., 4° C. and 37° C.) for 0, 1, 3 and 7 days in PBS containing 10% glycerol. At last, TCID₅₀ was used to titrate the remaining infectivity.

### Example 1. Expression of pV During BAdV-3 Infection

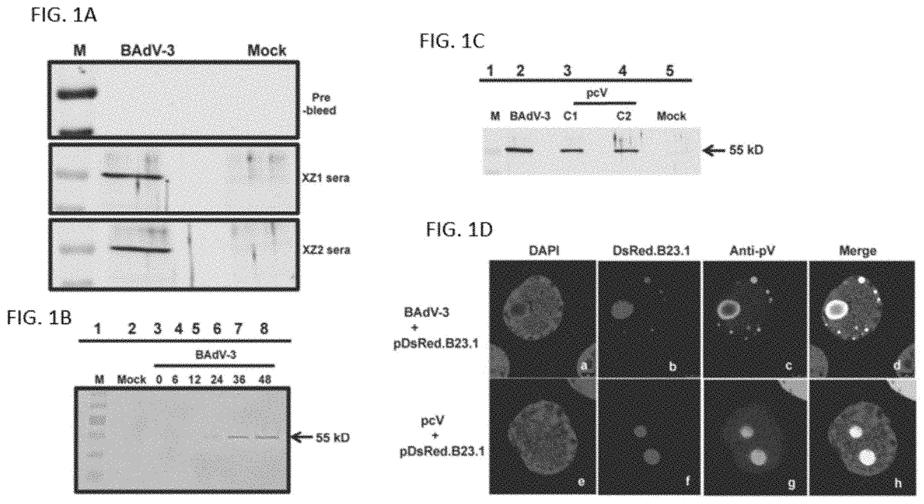

To characterize BAdV-3 pV, peptides ZX1 (¹MASSRLIKEEMLDIVAPEIYKRKR²⁴ (SEQ ID NO:16)) and peptide ZX2 (¹⁸⁰SRKRGVGKVEPTIQVLASKKRRMA²¹² (SEQ ID NO:17)) were synthesized and used to generate anti-pV sera designated as XZ1 and XZ2 sera, respectively. The specificity of the sera was analyzed by Western blot using BAdV-3 infected MDBK cells. As seen in FIG. 1A, both XZ1 serum and XZ2 serum detected a protein of 55 kDa in BAdV-3 infected cells. No such protein could be detected in mock-infected cells using XZ1 or XZ2 sera or BAdV-3 infected cells using pre-bleed sera. The protein could be detected at 24-48 hrs post infection (FIG. 1B lanes 6-8) but not at 12 hrs post infection (FIG. 1B lane 5).

Similarly, anti-pV pooled sera detected a 55 kDa protein in HEK293T cells transfected with plasmid pcDNA3-pV (pcV) (FIG. 1C, lanes 3-4) DNA. No such protein could be detected in plasmid pcDNA3 DNA transfected cells (FIG. 1C, lane 5).

### Subcellular Localization of pV

To determine the subcellular localization of pV, CRL cells were transfected with plasmid pDsRed. B23 (Gomez Corredor and Archambault, 2009) and infected with BAdV-3 at 48 hrs post-transfection. At 24 hrs post-infection, the cells were analyzed by indirect immunofluorescence assay using anti-pV serum. As seen in FIG. 1D, pV colocalized predominantly with nucleolar protein B23 fused to DsRed (pDsRed. B23) suggesting that pV localizes in the nucleolus of the infected cells. To determine if nucleolar localization is dependent on other viral proteins, we determined the localization of pV in VERO cells co-transfected with plasmid pcV and pDsRed. B23 DNAs by florescence microscopy. As seen in FIG. 1D, pV co-localizes predominantly with nucleolar marker B23 fused to DsRed in the nucleolus of the co-transfected cells.

### Example 2. Identification of pV Nucleolar Localization Signal

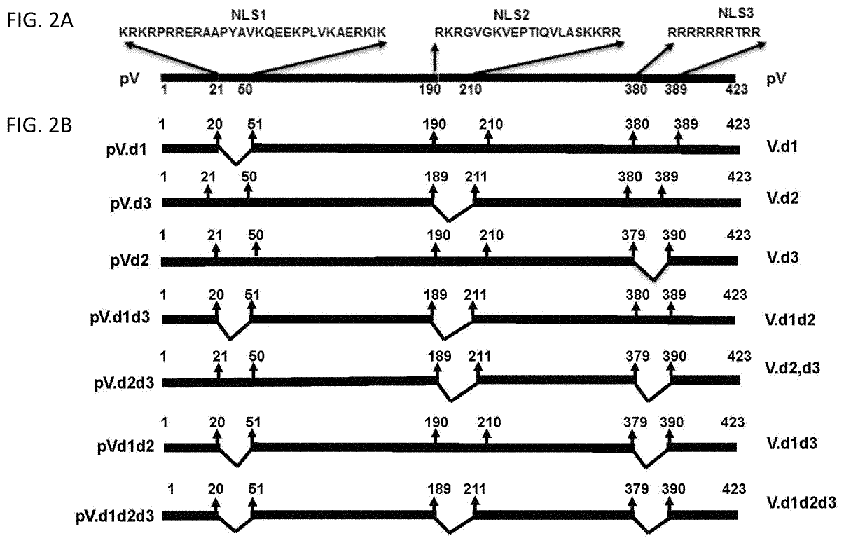

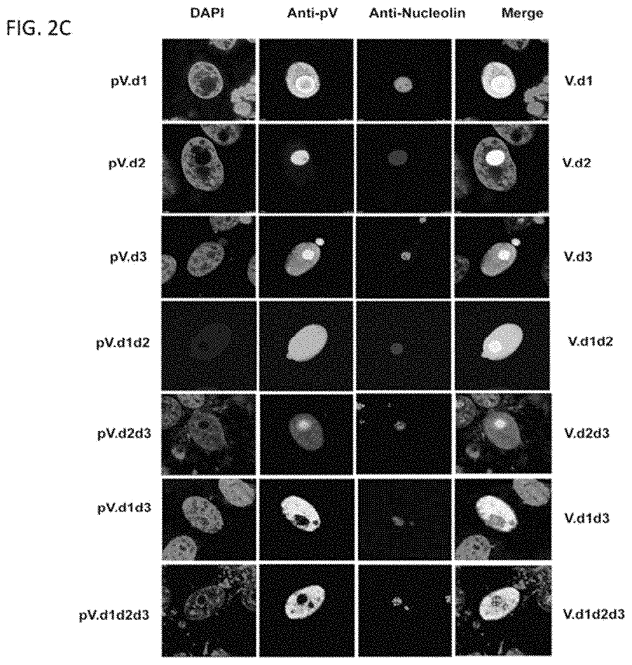

Bioinformatic analysis of pV protein sequence using motif prediction algorithms by such as “PredictProtein” predicted that the amino acids ²¹KRKRPRRERAAPYAVKQEEKPLVKAERKIK⁵⁰ (SEQ ID NO:18), ¹⁹⁰RKRGVGKVEPTIQVLASKKRR²¹⁰ (SEQ ID NO:19) and ³⁸⁰RRRRRRRTRR³⁸⁹ (SEQ ID NO:20) of BAdV-3 pV may act as potential nuclear localization signals (NLSs) (FIG. 2A). To determine if these domains act as NLS, we constructed plasmids expressing mutant pV containing specific NLS domain deletions (FIG. 2B). Vero cells co-transfected with plasmid pDsRed. B23 DNA and individual plasmid DNA expressing mutant pV protein were analyzed with immunofluorescence assay at 48 hrs post transfection. As seen in FIG. 2C, the mutant pV containing deletion of amino acid 21-50 (V.d1) localized both in the nucleus and the nucleolus of the transfected cells. Similarly, mutant pV containing deletion of amino acid 380-389 (V.d3) localized both in the nucleus and the nucleolus of the transfected cells. However, mutant pV containing deletion of amino acid 190-210 (V.d2) localize to the nucleolus of the transfected cells. Interestingly, mutant pV containing a deletion of amino acids 21-50 and 190-210 (v.d1d2) or deletion of amino acids 190-210 and 380-389(V.d2d3) could be detected in the nucleus and nucleolus of the transfected cells. In contrast, mutant pV containing the deletion of amino acids 21-50 and 380-389 (pV.d1d3), or deletion of amino acids 21-50,190-210 and 380-389 (V.d1d2d3) localized predominantly in the nucleus of the transfected cells.

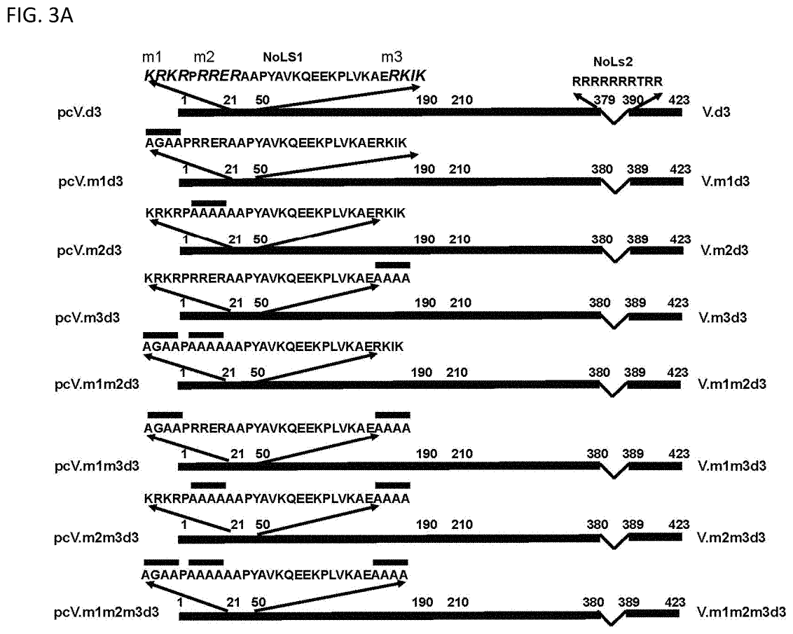

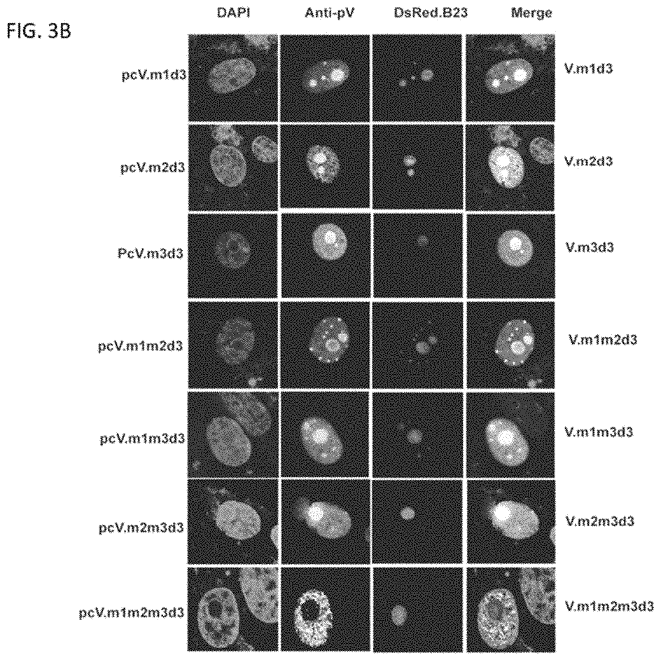

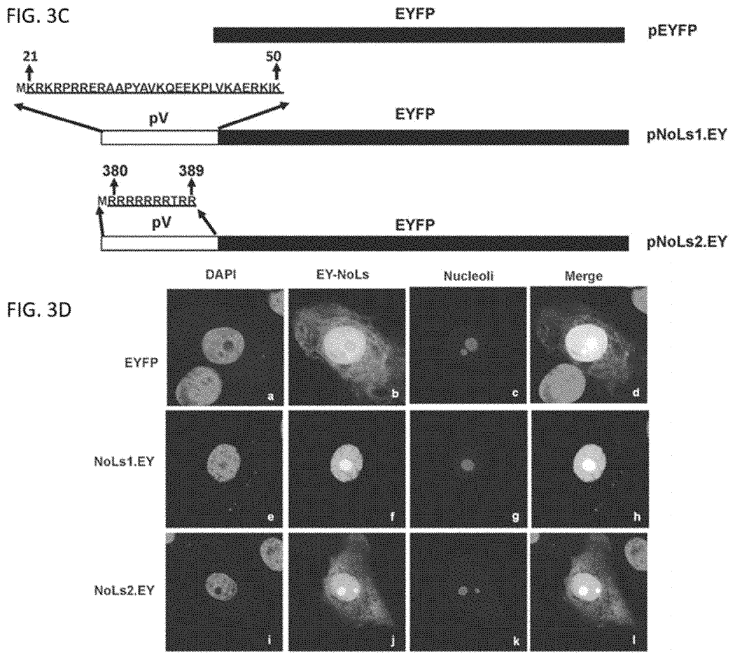

Earlier, Weber et al (Weber et al., 2000) suggested that the basic amino acid rich sequence K/R-K/R—X-K/R (SEQ ID NO:21), wherein X stands for any amino acids, may play a role in protein nucleolar localization. Our analysis of nucleolar localization sequences (NoLSs) NoLS1 (amino acid 20-50) and NoLS2 (amino acid 380-389) sequence identified three motifs (²¹KRKR²⁴ (SEQ ID NO:22), ²⁶RRER²⁹ (SEQ ID NO:23) and ⁴⁷RKIK⁵⁰ (SEQ ID NO:24)) in NoLS1, which have the potential to act as NoLs (FIG. 3A). To determine the role of each motif (m1, m2, m3) in nucleolar localization, we constructed a panel of plasmids expressing mutant pV proteins (amino acid 380-389 deleted) in which the basic residues of identified potential NoLs motifs were replaced with glycine\alanine residues (FIG. 3A). Vero cells were co-transfected with plasmid pDsRed. B23 and individual plasmid DNA expressing mutant pV protein, and analyzed with immunofluorescence assay using anti-pV sera. As seen in FIG. 3B, pV is predominantly localized in the nucleolus of cells transfected with plasmids expressing mutant pV containing deletion of amino acid substitution in single motif (V.m1d3, V.m2d3, V.m3d3,) or double motif (V.m1m2d3, V.m2m3d3, V.m1m3d3). In contrast, pV is predominantly localized in the nucleus of the cells transfected with plasmid expressing mutant pV containing basic amino acid substitution in all three potential NoLS (v.m1m2m3d3). To confirm the nucleolar retention function of BAdV-3 pV amino acids 21-50 and 380-389, DNA fragments encoding amino acids 21-50 and 380-389 were fused in-frame with enhanced yellow fluorescent protein (EYFP) gene to create plasmids pNoLs1. EY and pNoLs2. EY expressing fusion proteins (FIG. 3C). Vero cells co-transfected with plasmid pDsRed. B23 DNA and either plasmid pNoLs1. EY DNA or plasmid pNoLS2. EY DNA were analyzed by confocal microscopy at 48 hrs post transfection. As seen in FIG. 3D, EYFP was detected both in the nucleus and in the cytoplasm of the transfected cells as EYFP could diffuse passively in the nucleus due to its small size (26 kDa). In contrast, NoLS1 (amino acids 21-50) and NoLS2 (amino acids 380-389) were able to direct the heterologous protein (EYFP) predominantly to the nucleus and the nucleolus of the transfected cells.

### Example 3. Identification of pV Nuclear Localization Signal

To determine the nuclear localization signal(s) of BAdV-3 pV, we constructed plasmids expressing mutant BAdV-3 pV containing truncations and/or internal deletions (FIG. 4A). Vero cells were transfected with individual recombinant plasmid DNA. At 48 hrs post transfection, transfected cells were analyzed by indirect fluorescence using antipV sera. As seen in FIG. 4B, mutant pV containing deletions of amino acids 191-423 (V.d4,V.d5,V.d6 and V.d7) appear to be localized in the nucleus of the transfected cells. Moreover, mutant pV containing deletion of amino acid 21-50 and 190-423 (V.d8) also localized to the nucleus of the transfected cells. As expected, V.d4, V.d5 and V.d7 (contain one or both identified NoLS) localized to the nucleolus of the transfected cells, while V.d6 (absence of identified NoLs1 and 2) localized to the nucleus of the transfected cells. In contrast, analysis of mutant V.d9 (containing deletion of amino acids 21-50+101-210+380-423), V.d10 (containing deletion of amino acid 2-100+190-210+380-423) and V.d11 (containing deletion of amino acids 21-50+81-120+190-210+380-423) suggested that amino acids 21-50, 81-120, 190-210 and 380-423 may contain nuclear localization signal(s) motifs which may have redundant function. To further determine the importance of these sequences in nuclear localization of pV, we constructed plasmids (FIG. 4C) containing deletion of three of the four regions of pV containing potential NLS. Vero cells were transfected with individual plasmid DNAs and subcellular localization of mutant pVs were analyzed at 48 hrs post transfection by indirect immunofluorescence using anti-pV sera. As seen in FIG. 4D, mutant pV containing amino acids 81-120 (V.d12), 190-210 (V.d13) or amino acid 380-423 (V.d14) localized to nucleus\nucleolus. In contrast, mutant pV containing amino acid 21-50 (V.d15) localized predominantly in the cytoplasm of the transfected cell.

To examine if pV NoLSs can serve as NLSs, plasmids expressing pV.d16 (containing deletion of amino acids 81-120+190-210+390-423) (FIG. 4C) and NoLs1-GFPβGal (FIG. 4E) were constructed and used to transfect Vero cells. As shown in FIGS. 4D and 4F, pV.d16 and NoLs1-GFPβGal were localized in nucleus and cytoplasm, respectively.

### Example 4. Interaction of pV with Importins

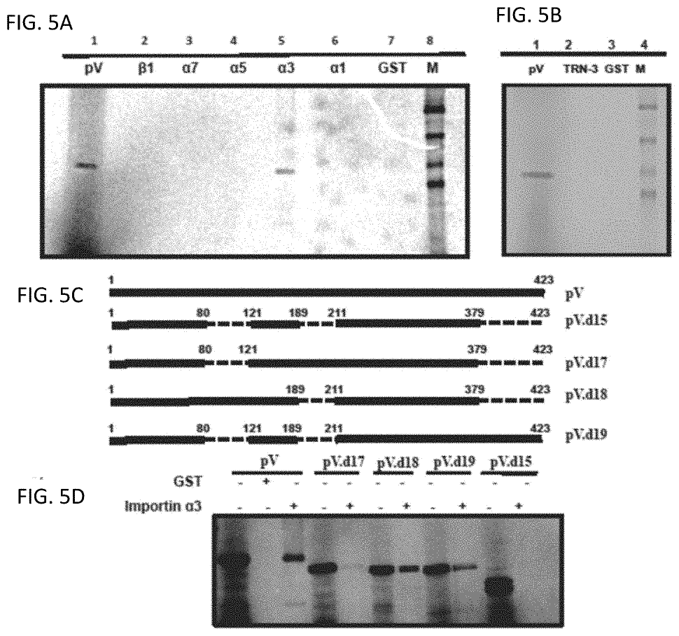

Members of the importin super family play an important role in nuclear transport of proteins. Since transport of some adenovirus proteins requires importins (Kohler et al., 1999; Kulshreshtha et al., 2014; Paterson et al., 2012; Wodrich et al., 2006), we performed a GST pull down assay using purified GST-fusion proteins of importin α1, importin α3, importin α5, impotin α7 or importin β1 individually immobilized on glutathione-sepharose beads with radiolabelled in vitro synthesized BAdV-3 pV. As seen in FIG. 5A, GST-importin α3 was able to bind radiolabelled pV (lane 5) as similar protein was observed in input protein control (lane 1). No radiolabelled pV was observed when purified GST alone (lane 7) or GST fusions of importin β1 (lane 2), importin α7 (lane 3), importin α5 (lane 4) or importin α1 (lane 6) bound to glutathione-sepharose beads were used in pull down assays.

Like pV (FIG. 5C,D), GST-importin α3 bound to glutathione-Sepharose was able to bind radiolabeled pV.d18 (deletion of amino acids 190-210 and 380-423), pV.d19 (deletion of amino acids 81-120 and 190-210) and pV.d17 (deletion of amino acids 81-120 and 380-423) albeit with less intensity. However, no such interaction was observed when GST-importin α3 fusion bound to glutathione-sepharose was used to pull down pV.d15 containing deletion of amino acids 81-120, 190-210 and 380-423.

Recently, we demonstrated that BAdV-3 33K interacts with transportin-3 (Kulshreshtha et al., 2014). To determine if pV binds to transportin-3 (Hindley et al., 2007; Kulshreshtha et al., 2014), GST pull down assay was performed using GST alone or GST-Transportin fusion protein and in vitro [35S] methionine labeled pV. As seen in FIG. 5B, a protein could be observed in input protein control (lane 1). However, no similar protein could be detected bound to GST-TRN-SR2 (transportin-3) fusion protein (Lane 2) or GST alone (lane 3).

### Example 5. Construction and Localization of BAdV-3s Expressing Mutant pV Proteins

### Materials and Methods—Isolation of pV Nucleolar Localization Signal BAdV-3 Mutants

To isolate mutant BAdV-3s, we constructed full length BAdV-3 plasmids containing mutant BAdV-3 genomic DNAs as described (Chartier et al., 1996).

a) Plasmid pUC304a.pVd1.

A 972 bp DNA fragment was amplified by PCR using primers M-F and d(21-50)-F1-R (Table 1), and plasmid pcDNA3-pV DNA as the template. Similarly, an 1134-bp DNA fragment was amplified by PCR using primers d(21-50) F2-F and pV-XhoI-R (Table 1), and plasmid pcDNA3-pV DNA as the template. In the third PCR, these two PCR fragments were annealed and used as DNA template to amplify a 2068-bp DNA fragment by overlapping PCR using primers M-F and pV-XhoIR (Table 1). A 1171-bp EcoRI-XhoI DNA fragment of the final PCR product (2068 bp) was isolated and ligated to EcoRI-XhoI digested plasmid pcDNA3 to create plasmid pcDNA3-pV-d(21-50). A 528-bp EcoRI-NheI DNA fragment of plasmid pcDNA3-pVd(21-50) was isolated and ligated to EcoRI-NheI digested pMCS.pV to create plasmid pMCS.pVd1.

TABLE 1
List of primers

| | |
|---|---|
| M-F | 5-TCTGCTCTGA |
| | TGCCGCATAG |
| | TTAAGCC-3 |
| | (SEQ ID NO: 3) |
| d(21-50)- | 5-CGCTTTCTAG |
| F1-R | AGCCGCGGTA |
| | AATCTCAGGC |
| | GCCACGATGT |
| | C-3 |
| | (SEQ ID NO: 4) |
| d(21-50- | 5-TCGTGGCGCC |
| F2-F | TGAGATTTAC |
| | CGCGGCTCTA |
| | GAAAGCGGGC |
| | CTTG-3 |
| | (SEQ ID NO: 5) |
| pV-XhoI-R | 5-AATACTCGAG |
| | AGCGCTTAAC |
| | GGCGGAGCCG |
| | GGTTAC-3 |
| | (SEQ ID NO: 6) |
| M12-F1-R | 5-CTGCAGCAGC |
| | TGCTGCGGGT |
| | GCAGCTCCTG |
| | CGTAAATCTC |
| | AGGCGCCACG |
| | ATG-3 |
| | (SEQ ID NO: 7) |
| M12-F2-F | 5-CGCAGGAGCT |
| | GCACCCGCAG |
| | CAGCTGCTGC |
| | AGCACCGTAT |
| | GCTGTGAAG-3 |
| | (SEQ ID NO: 8) |
| M3-F1-R | 5-TTTCTAGAGC |
| | CGCGAGCAGC |
| | TGCTGCCTCC |
| | GCCTTTACTA |
| | AAGGCTTCTC |
| | -3′ |
| | (SEQ ID NO: 9) |
| M3-F2-F | 5-TTAGTAAAGG |
| | CGGAGGCAGC |
| | AGCTGCTCGC |
| | GGCTCTAGAA |
| | AGCGGGCCTT |
| | G-3 |
| | (SEQ ID NO: 10) |
| pV-EcoRI-F | 5-GGAGCCGAAT |
| | TCATGGCCTC |
| | CTCTCGGTTG |
| | ATTAAAGAA |
| | G-3 |
| | (SEQ ID NO: 11) |
| pV-d | 5-CAGCGCTGAG |
| (380-389) | GCGGGGAGTC |
| F1-R | GCGACTGCAG |
| | GCAGGCGCAC |
| | AC-3 |
| | (SEQ ID NO: 12) |
| pV-d | 5-GTGTGCGCCT |
| (380-389) | GCCTGCAGTC |
| F2-F | GCGACTCCCC |
| | GCCTCAGCGC |
| | TG-3 |
| | (SEQ ID NO: 13) |
| dV-F2-R | 5-GTCCATGGCG |
| | TGTTAACAAG |
| | CTGTG-3 |
| | (SEQ ID NO: 14) |

At last, a 6.2-kb EcoRV-Bst1107I fragment of plasmid pMCS.pVd1 was isolated and recombined with Shill digested plasmid pUC304a.dV DNA in Escherichia coli BJ5183 (Chartier et al., 1996) to generate plasmid pUC304a.pVd1.

b) Plasmid pUC304a.pVm123.

A 986-bp DNA fragment was amplified by PCR using primers M-F and M12-F1-R (Table 1) and plasmid pcDNA3-pV DNA as the template. Similarly, a1025-bp DNA fragment was amplified by PCR using primers M12-F2-F and pV-XhoI-R (Table 1), and plasmid pcDNA3-pV DNA as the template. In the third PCR, two fragments were annealed and used to amplify a 2159-bp DNA fragment by overlapping PCR using primers M-F and pV-XhoI-R (Table 1). At last, a 1261-bp EcoRIXhoI DNA fragment of the PCR product (2159 bp) was isolated and ligated to EcoRIXhoI digested plasmid pcDNA3 to generate plasmid pcDNA3-pV-m12.

To create pcDNA3-pV-m123, a 1059-bp DNA fragment was amplified by PCR using primers M-F and M3-F1-R (Table 1), and plasmid pcDNA3-pV-m12 as the template. An 1141-bp DNA fragment was amplified by PCR using primers M3-F2-F and pV-XhoI-R (Table 1), and plasmid pcDNA3-pV-M12 DNA as the template. In the third PCR, these two DNA fragments were annealed and used to amplify a 2159-bp DNA fragment by overlapping PCR using primers M-F and pV-XhoI-R (Table 1). At last, a 1261-bp DNA fragment of the PCR product (2159-bp) was isolated and ligated to EcoRIXhoI digested plasmid pcDNA3 to generate plasmid pcDNA3-pV-m123.

A 618-bp EcoRI-NheI fragment of plasmid pcDNA3-pV-m(1+2+3) was isolated and ligated to EcoRI-NheI digested plasmid pMCS.pV to create plasmid pMCS.pVm123. The SbfI digested plasmid pUC304-dV was recombined with a 6.3-kb EcoRV-Bst1107I DNA fragment of plasmid pMCS.pVm123 in Escherichia coli BJ5183 (Chartier et al., 1996) creating plasmid pUC304a.pVm123.

c) Plasmid pUC304a.pVd3 and pUC304a.pVd1d3.

An 1171-bp fragment was amplified by PCR using primers pV-EcoRI-F and F1-R (Table 1), and plasmid pMCS.pV DNA as the template. Similarly, a 661-bp fragment was amplified by PCR using primers pVd(380-389) F2-F and dV-F2-R (Table 1), and plasmid pMCS.pV DNA as the template. In the third PCR, two PCR fragments were annealed and used to amplify a 1790-bp DNA fragment by overlapping PCR using primers pV-EcoRI-F and dV-F2-R (Table 1). At last, a 650-bp SacI-HpaI fragment of PCR product (1790 bp) was isolated and ligated to SacIHpaI digested plasmid pMCS.pV and pMCS.pVd1 to create plasmid pMCS.pVd3 and pMCS.pVd1d3, respectively.

The SbfI digested plasmid pUC304a.dV was recombined with a 6.2-kb EcoRVBst1107I fragment of plasmid pMCS.pVd3 or plasmid pMCS.pVd1d3 in Escherichia coli BJ5183 (Chartier et al., 1996) to generate plasmid pUC304a.pVd3 and plasmid pUC304a.pVd1d3, respectively.

### Results

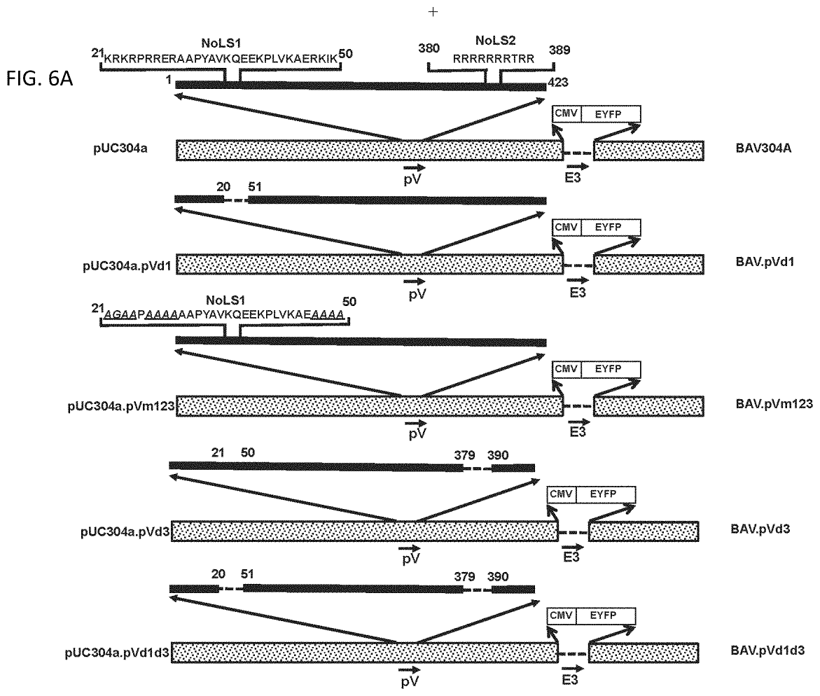

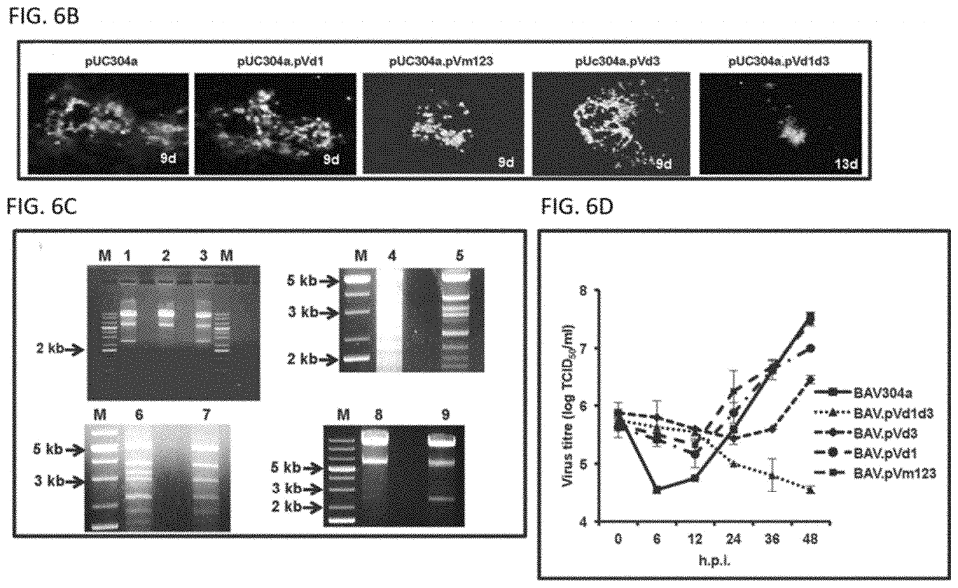

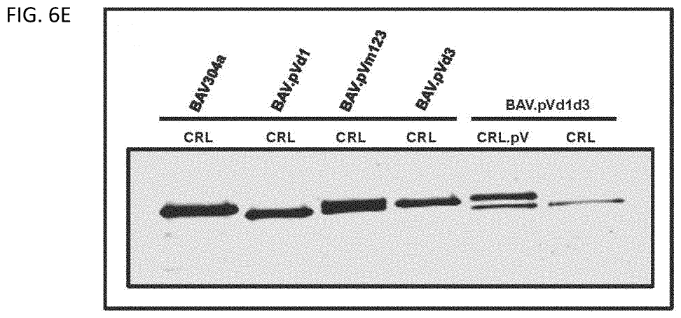

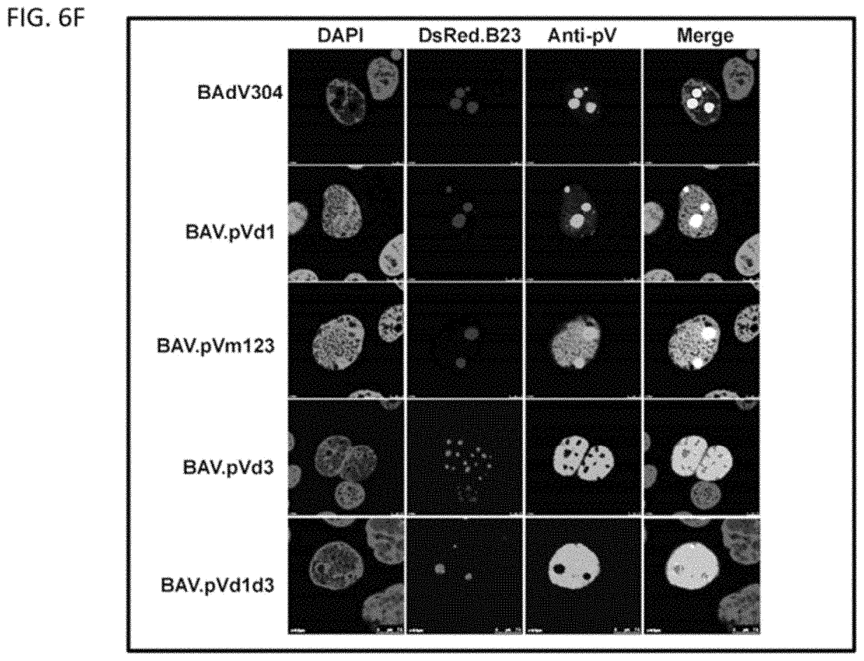

To determine if the potential NoLSs are required for efficient replication of BAdV-3, we constructed full length plasmid genomic clones expressing mutant pV containing deletion of potential NoLSs and \ or substitutions of basic residues with alanine\glycine of potential NoLS1 (FIG. 6A). Monolayer of VIDO DT1 (cotton rat lung cells expressing I-Scel recombinase) cells were transfected with 5-7.5 μg of individual plasmid DNAs. The cytopathic effects appeared between 9-15 days (FIG. 6B). However, repeated transfection of VIDO DT1 (cotton rat lung cells expressing I-SceI recombinase) cells with plasmid pUC304a.pVd1d3 did not produce any cytopathic effects. Moreover, reinfection of fresh VIDO DT1 (cotton rat lung cells expressing I-SceI recombinase) with supernatants of infected cell lysates containing mutant viruses (FIG. 6A) named BAV.pVd1 (deletion of amino acid 21-50), BAV.pVm123 (containing substitutions of basic residues of all three motifs of amino acid s21-50) and BAV.pVd3 (containing deletion of amino acids 380-389) produced infectious virions. In contrast, reinfection of fresh VIDO DT1 (cotton rat lung cells expressing I-SceI recombinase) with supernatant of cell lysates potentially containing mutant BAV.pVd1d3 (containing deletion of amino acid 21-50 and amino acid 380-389) did not produce any infectious virion (Data not shown). To produce mutant BAV.pVd1d3, CRL. PV cells (CRL cells expressing BAdV-3 pV) were transfected with 5-7.5 μg of PacI digested plasmid pUC304a.pVd1d3 DNA. The cytopathic effects were observed in 13 days (FIG. 6B). To purify the mutant viruses, the MDBK cells infected individually with BAV.pVd1, BAV.pVm123 or BAV.pVd3 (FIG. 6A) or CRL. PV cells infected with BAV.pVd1d3 were collected, freeze-thawed and purified by CsCl density gradient purification.

The presence of the desired mutations was confirmed by DNA sequencing and restriction enzyme digestion of virion DNAs. Since an additional XbaI recognition site was introduced into mutant BAV.pVd1 or BAV.pVm123 genomes, the viral genomes were digested with XbaI. As seen in FIG. 6C, BAV.pVd1 (Lane 1) and BAV.pVm123 (lane 3) genomes had a band of 2.4 kb, which was missing in BAV304a (lane 2). Similar analysis of XbaI digested BAVd1d3 (lane 9) genome detected an expected band of 2.4 kb, but not in BAV304a (lane 8). Since an additional Pst1 recognition site was introduced to the viral genome BAV.pVd3, the viral genome was digested with Pst1. As seen in FIG. 6C, a 3.2 kb band was detected in BAV304a (lane 5) but not in BAV.pVd3 (lane 4). Similar analysis of Pst1 digested BAV.pVd1d3 genome detected an expected band of 3.2 kb in BAV304a (lane 6) but not in BAV.pVd1d3 (lane 7).

The ability of the mutant BAdV-3s to express pV protein was analyzed by Western blot analysis of proteins from the lysates of virus infected cells. As seen in FIG. 6E, protein bands of expected molecular weight could be detected in lysates of CRL cells infected with mutant BAV.pVd1, BAV.pVm123, BAV,pVd3 or BAV.pVd1d3. The ability of mutant BAV.pVd1d3 to express pV protein was analyzed in CRL. PV (CRL expressing BAdV-3 pV) cells. As expected, two proteins of 55 kDa (representing wildtype pV expressed in CRL.pV cells) and 53 kDa (representing mutant pV expressed in BAV.pVd1d3) were detected in lysates of CRL. PV cells infected with BAV.pVd1d3.

### Sub Cellular Localization of Mutant pV Protein in Recombinant BAdV-3 Infected Cells.

To determine the effect of deletions or amino acid substitutions on nucleolar localization of pV, CRL cells were transfected with plasmid pDsRed. B23 DNA. At 48 hrs post transfection, the cells were infected with BAV304a or individual mutant BAdV-3s. At 24 hrs post infection, the cells were analyzed by immunofluorescence using antipV sera. As seen in FIG. 6F, pV appears localized mainly in the nucleoli of BAV304a or BAV.pVd1 infected cells. Similarly, pV appears localized predominantly in the nucleoli of BAV.pVm123 infected cells. In contrast, pV appears localized in the nucleus of BAV.pVd3 or BAV.pVd1d3 infected cells.

### Growth Kinetics of Viruses

To examine if the deletion\mutation of pV nucleolar localization signals affects BAdV-3 replication, we compared the ability of the mutant viruses and BAV304a to grow on MDBK cells. The virus infected cells were harvested at indicated time points post infection, freeze-thawed 3-5 times and cell lysates were used to determine the virus titers by TCID₅₀ assay. As seen in FIG. 6D, at 48 hrs, virus titer of mutant BAVpVd1, BAV.pVm123, BAV.pVd3 appeared 0.6 to 1.0 log less compared to BAV304a. In contrast, mutant BAV.pVd1d3 did not replicate in MDBK cells.

### Example 6. Analysis of Gene Expression in Mutant Virus-Infected Cells

### Materials and Methods—Antibodies

The production and characterization of antibodies raised against BAdV-3 DBP (Zhou et al., 2001), fiber (Wu and Tikoo, 2004) and 100K (Makadiya et al., 2015) have been described. Anti-hexon serum detects a protein of 98 kDa in BAdV-3 infected cells (Kulshreshtha et al., 2004). Anti-pVII serum detects two proteins of 22 and 20 kDa in BAdV-3 infected cells (Paterson, 2010). Anti-pX recognizes a protein of 25 kDa in BAdV-3 infected cells.

### Materials and Methods—Protein Expression Analysis

Monolayers of MDBK in six-well plate were infected with BAV304a or mutant BAdV-3s at multiplicity of infection of 1. At 24 h post-infection, infected cells were harvested and probed by Western blotting using protein-specific rabbit antisera and mouse anti-β-actin as primary antibodies (Sigma), Alexa Fluor 680 goat anti-rabbit (Invitrogen) and IRDye 800 goat anti-mouse (Rockland), respectively, as secondary antibodies. At last, the membranes were imagined and analyzed by using the Odyssey® CLx Imaging System (LI-COR).

### Results

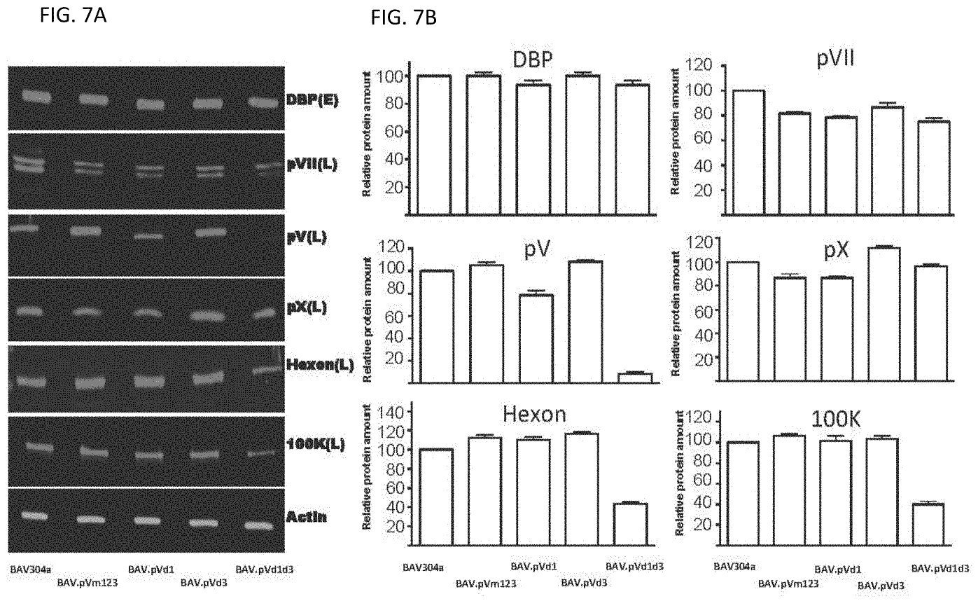

Since the deletion\mutation of pV NoLS influences the viral growth kinetics, we investigated the effects of pV NoLS deletion/mutations on the expression of early and late proteins in mutant BAdV-3 infected cells by Western blot using protein specific antisera. As seen in FIG. 7A, anti-DBP serum, anti-pVII serum, anti-pV serum, anti-pX serum, anti-hexon serum and anti-100K serum detected proteins of expected molecular weights both in BAV304a and mutant virus infected cells. Densitometer analysis of protein production (FIG. 7B) showed no significant differences in the expression of DBP, pX in any mutant BAdV-3 infected cells compared to BAV304a infected cells. Similarly, there was no dramatic change in the expression of pVII in mutant infected cells compared to BAV304a infected cells. However, expression of hexon and 100K was significantly reduced in BAV.pVd1d3 infected cells compared to BAV304a infected cells. Moreover, the expression of pV was severely reduced in mutant BAVd1d3 infected cells compared to BAV304a, BAV.pVd1, BAV.pVm123, and BAV.pVd3 infected cells.

### Example 7. Structural Protein Incorporation Assay

### Materials and Methods—Antibodies

The production and characterization of antibodies raised against BAdV-3 DBP (Zhou et al., 2001), fiber (Wu and Tikoo, 2004) and 100K (Makadiya et al., 2015) have been described. Anti-hexon serum detects a protein of 98 kDa in BAdV-3 infected cells (Kulshreshtha et al., 2004). Anti-pVII serum detects two proteins of 22 and 20 kDa in BAdV-3 infected cells (Paterson, 2010). Anti-pX recognizes a protein of 25 kDa in BAdV-3 infected cells.

### Results

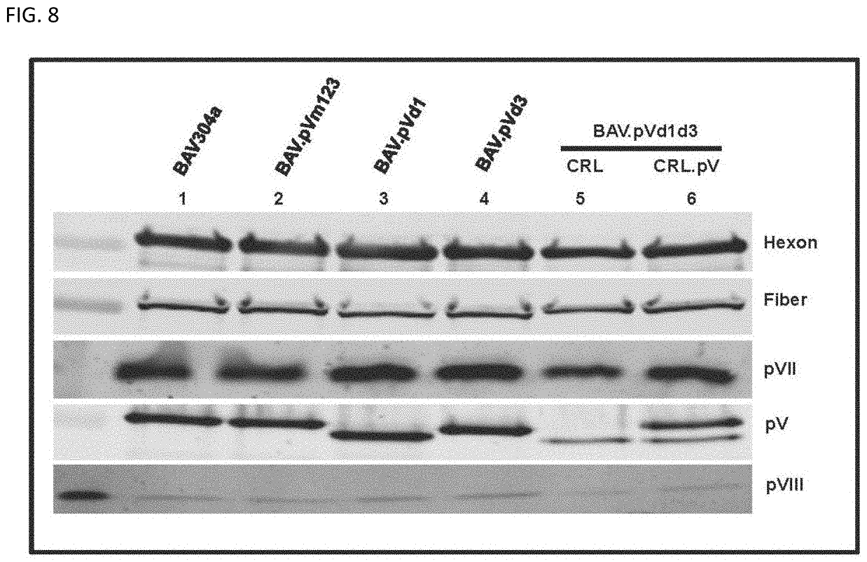

To determine if the decreased late protein express influences the structural protein incorporation, structural proteins in purified virus were separated by 10% SDS-PAGE, transferred to nitrocellulose membrane and probed with western blot by anti-Hexon (Kulshreshtha et al., 2004), anti-Fiber (Wu and Tikoo, 2004), anti-pVII (Paterson, 2010), anti-pV and anti-pVIII (Ayalew, 2014) antisera. As shown in FIG. 8, there was no detectable difference between Hexon, Fiber, pVII and pVIII expression among these recombinant BAdV-3s. However, the expression of pV mutants was different. Firstly, different sized pV mutants were detected from different recombinant BAdV-3s, indicating the deletion or mutation of pV NoLS(s). Secondly, different pV expression patterns shown in BAV.pVd1d3 purified from CRL and CRL.pV cells. Two pV bands were detected in BAV.pVd1d3 purified from CRL.pV cells, while only the lower one was detected in the BAV.pVd1d3 purified from CRL cells.

### Example 8. Analysis of BAdV-3 Capsid Assembly

### Materials and Methods—Transmission Electron Microscopy

Monolayers of MDBK cells were infected with BAV304a or BAV.pVd1d3 at MOI of 5. At 48 h post-infection, the cells were harvested and fixed in 2.5% glutaraldehyde in 0.1 M PBS, followed by post-fixation in 1% OsO4 and dehydration in a graded ethanol series and propylene oxide. Dehydrated cells were infiltrated in mixtures of propylene oxide and EMbed-812 embedding medium, and then polymerized in embedding capsules at 60° C. for 24-48 h. At last, the pellet was sectioned with a Reichert ultracut microtome, each section stained with 2% uranyl acetate and viewed on a Philips CM10 TEM.

### Results

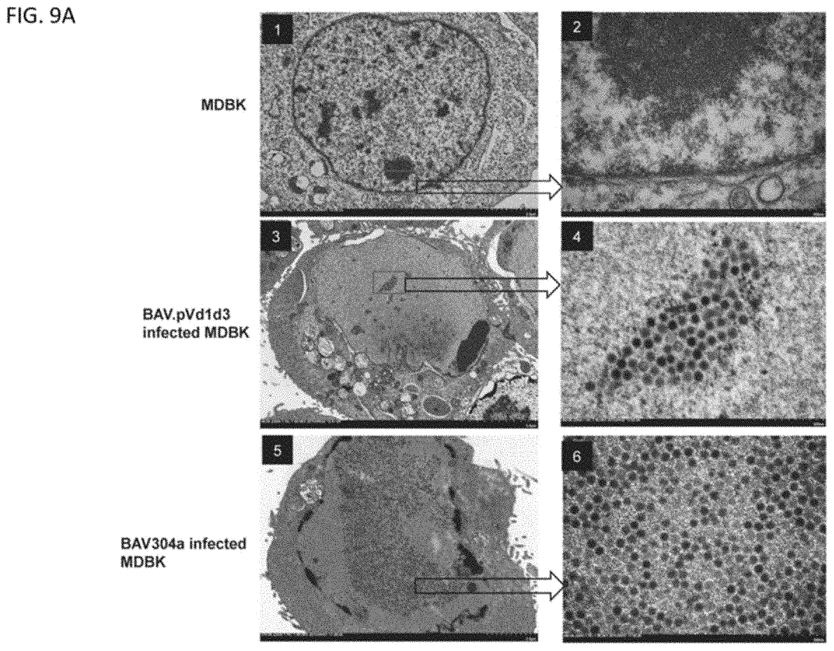

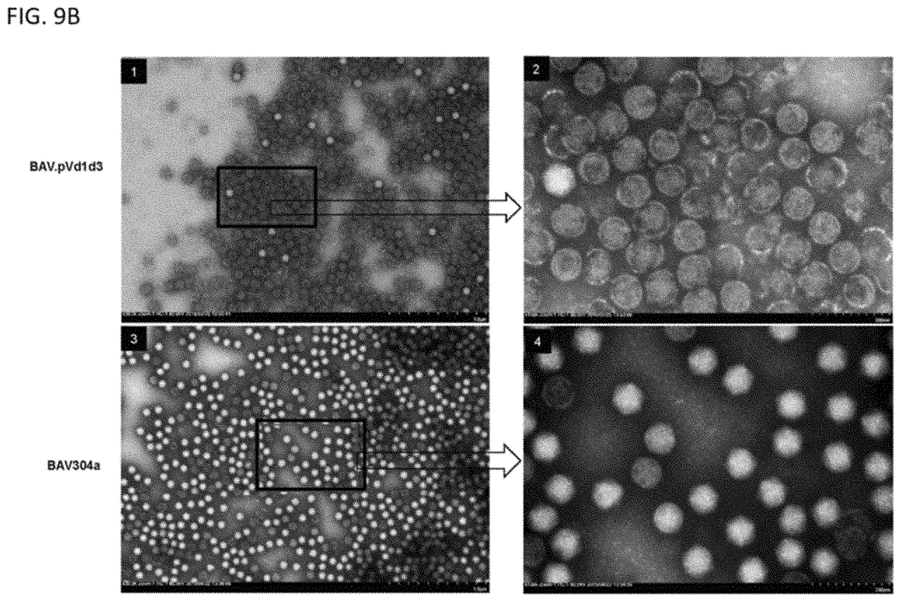

Since expression of BAdV-3 proteins (hexon, 100, and pV) was significantly reduced in NoLSs deleted BAdV-3 (BAV.pVd1d3), viral capsid assembly was analyzed initially in BAV.pVd1d3 and BAV304a infected MDBK cells. Capsid formation was analyzed in BAV.pVd1d3 infected cells by TEM. As seen in FIG. 9A, although capsid formation was observed in BAV.pVd1d3 infected cells, many of the capsids were lightly stained. Moreover, there were less amount of capsids observed in BAV.pVd1d3 infected cells than BAV304a infected cells.

Viral capsid assembly was analyzed by using BAV.pV1d3 and BAV304a viral particles purified from MDBK cells. The infected cells were harvested, freeze-thawed, and the virions were purified using CsCl gradients. As seen in FIG. 9B, few mature virions were present in purified BAV.pVd1d3. Moreover, the amount of mature virions present in purified BAV.pVd1d3 was significantly lower than the amount detected in BAV304a infected cells.

### Example 9. Thermostability of Recombinant BAdV-3s

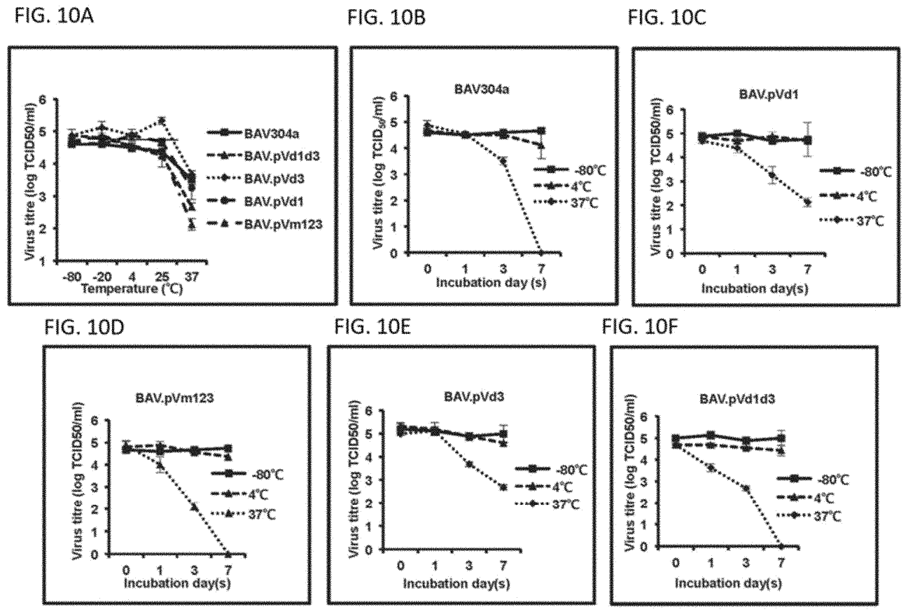

Deletions and mutations in viral genome are always associated with thermo vulnerability (Ugai et al., 2007). To examine if the deletion or mutation of pV NoLSs leads to the decrease of BAdV-3 thermostability, wild-type and recombinant BAdV-3s were treated as described above (Ugai et al., 2007). As seen in FIG. 10A, there is no titer difference when the temperature was under 25° C. However, when viruses were incubated at 37° C., the titers dropped significantly, especially for BAV.pVd1d3 and BAV.pVm123. To assess the different dynamics of viral inactivation, wild-type and recombinant BAdV-3s were treated at −80° C., 4° C. or 37° C. for 0, 1, 3 or 7 days. As seen in FIG. 10B-F, after seven days incubation at −80° C. or 4° C., there was no detectable change in all these five BAdV-3s. However, after seven days incubation at 37° C., BAV304a, BAV.pVm123 and BAV.pVd1d3 lost all their infectivity, while for the single NoLS deleted recombinant viruses BAV.pVd3 and BAV.pVd1, ˜103 infectious VP were still remaining.

### Example 10. Isolation of BAV.dV in CRL Cells

Materials and Methods—Construction of Plasmid pUC304A. dV

A 6.4-kb EcoRV-Bst1107I DNA fragment of plasmid pUC304A+(E3 deleted BAdV-3 containing CMV. EYFP inserted in E3 region), was isolated and ligated to a 2.1-kb EcoRV-Bst1107I fragment of plasmid pMCS1 (Thanbichler et al., 2007) creating plasmid pMCS-pV. To delete pV from pMSC-pV, a 465-bp fragment was amplified by using primers dV-F1-F: 5′-TGATCCGGTGGCCGACACAATCGAG-3′(SEQ ID NO:25); dV-F1-R: 5′-TGTGGCCGCTTGGCGGATGCCTGCAGGCACAGTGGGTTTATCGGCGCG-3′ (SEQ ID NO:26) and plasmid pMCS-pV DNA as a template. Similarly, a 602-bp fragment was amplified by PCR using primers dV-F2-F: 5′-GCCGATAAACCCACTGTGCCTGCAG GCATCCGCCAAGCGGCCACAGTAAC-3′ (SEQ ID NO:27); dV-F2-R: 5′-GTCCATGGCGTGTTAA CAAGCTGTG-3′ (SEQ ID NO:14) and plasmid pMCS-pV DNA as a template. In the third PCR, these two fragments were annealed and used as DNA temple to amplify the 1040-bp DNA fragment without pV by overlapping PCR using primers dV-F1-F and dV-F2-R. Finally, a 622-bp EcoRI-HpaI DNA fragment of the third PCR product was isolated and ligated to EcoRI-HpaI digested plasmid pMSC-pV creating plasmid pMSC.dV.

A 1.6-kb SbfI fragment (containing kanamycin resistant gene) of plasmid pUC4K (Taylor and Rose, 1988) was isolated and ligated to SbJI digested plasmid pMCS.dV to create plasmid pMSC-dV-Kan. The recombinant plasmid pUC304-dV-Kan was generated by homologous recombination in E. coli BJ5183 between the plasmid pUC304A+DNA and a 6.4-kb EcoRV-Bst1107I DNA fragment of plasmid pMCS-dV-Kan. Finally, plasmid pUC304.dV-Kan was digested with SbfI and large fragment was religated to create plasmid pUC304A.dV.

### Results

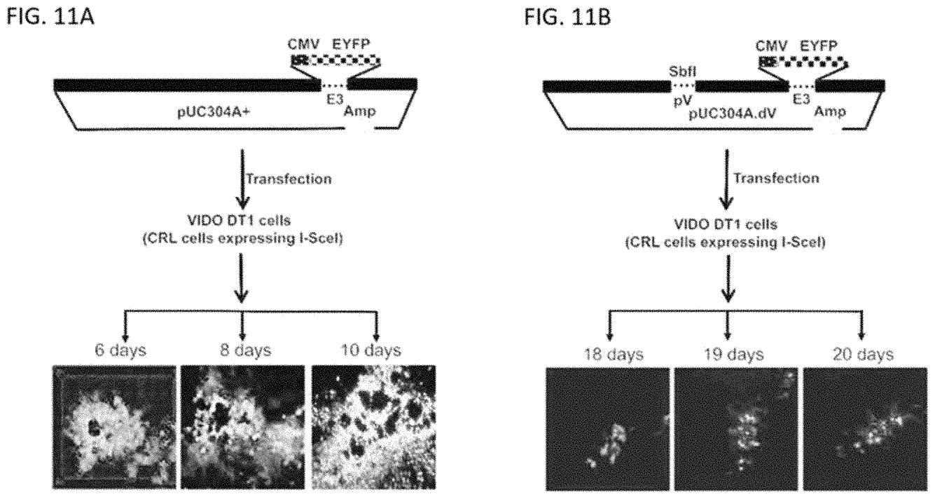

To determine if pV is essential for BAV304a (Du and Tikoo, 2010) replication, we constructed a plasmid pUC304A.dV containing BAdV-3 genome with deletion of pV and insertion of CMV-EYFP gene cassette in E3 deleted region (FIG. 11B). Individual plasmid pUC304A.dV or pUC304A+(containing BAdV-3 genome with insertion of CMV-EYFP gene cassette in E3 deleted region)(FIG. 11A) DNA were used to transfect VIDO DT1 cells.

At 6 days post-transfection, the EYFP expression and cytopathic effects were visible in the cells transfected with plasmid pUC304A+DNA (FIG. 11A). However, repeated transfection of VIDO DT1 cells with plasmid pUC304A.dV DNA did show EYFP expression in few cells but no any cytopathic effects even after 20 days post transfection (FIG. 11B). Moreover, while the lysates from the cells transfected with plasmid pUC304A+DNA produced cytopathic effects in freshly infected VIDO DT1 cells, the lysates from the cells transfected with plasmid pUC304A.dV DNA did not produce any cytopathic effect or expression of EYFP in freshly infected VIDO DT1 cells (data not shown). These results suggest that pV is essential for the replication of BAV304a.

### Example 11. Construction of CRL.pV Cells Expressing BAdV-3 pV

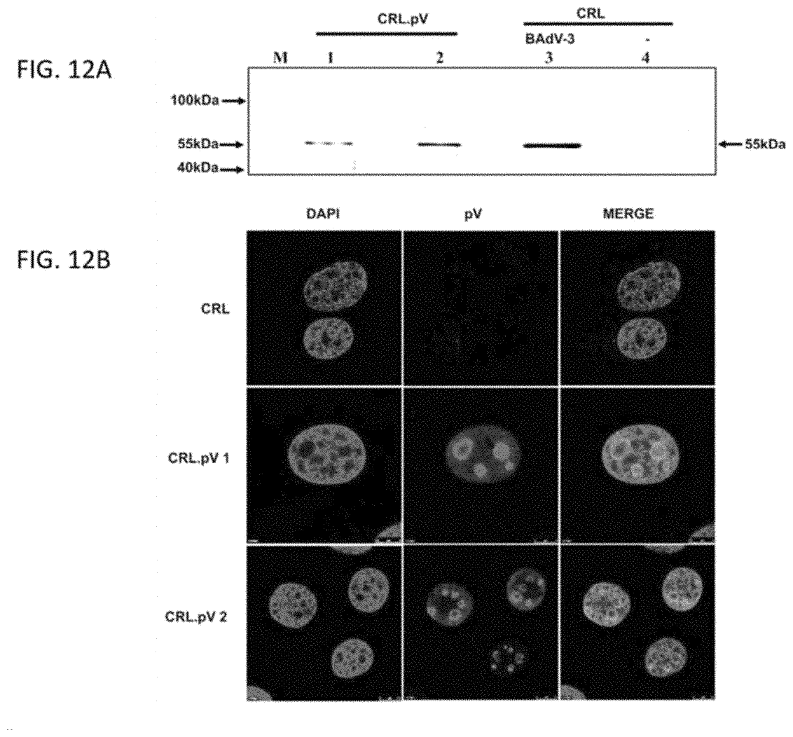

To isolate a cell line expressing BAdV-3 pV, CRL cells were transduced with lentivirus expressing BAdV-3 pV and grown in the presence of puromycin as described earlier (Du and Tikoo, 2010). The puromycin resistant clones were analyzed initially for the expression of pV by Western blot and immunofluorescence assay using pV specific antiserum. Earlier analysis using anti-pV serum suggested that pV is expressed as 55 kDa in BAdV-3 infected cells and localizes predominantly in the nucleolus of BAdV-3 infected cells (Zhao and Tikoo, 2016, manuscript in preparation). As shown in FIG. 12A, anti-pV serum detected a protein of 55 kDa in BAdV-3 infected cells. Similar protein could be detected in two puromycin resistant clones using anti-pV serum (FIG. 12A, lanes 1,2). No such protein could be detected in CRL cells (FIG. 12A, lane 4). Secondly, the sub cellular location of pV in puromycin resistant clones was analyzed by confocal microscopy. As seen in FIG. 12B anti-pV serum detected protein predominantly localized in the nucleolus of pV expressing cells (CRL.pV1, CRL.pV2). No such protein could be detected in the nucleolus of CRL cells.

### Example 12. Isolation of BAV.dV in CRL.pV Cells

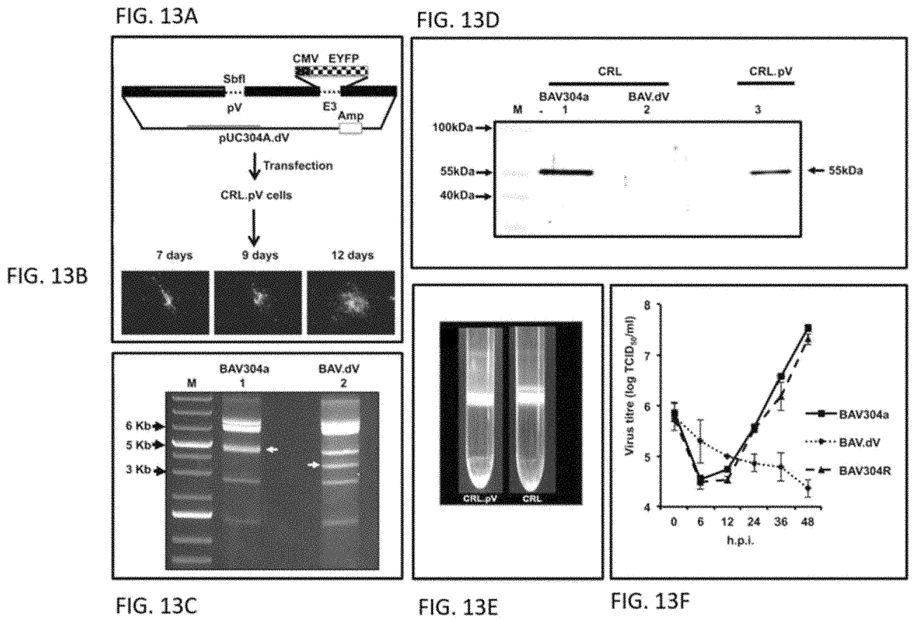

To isolate pV deleted BAV304a, CRL.pV cells were transfected with PacI digested plasmid pUC304A.dV DNA and observed for the development of cytopathic effects (FIG. 13A). As shown in FIG. 13B, the cytopathic effect and EYFP expression was firstly observed at 7 days post-transfection, which increased by day 12. To confirm the identity of recombinant virus named BAV.dV, first viral DNA was purified from infected CRL.pV cells, digested with KpnI and analyzed by agarose gel electrophoresis. As shown in FIG. 13C, BAV304a (Lane 1) contains a fragment of 4.7 kb, which was missing in BAV.dV. Instead, BAV.dV (lane 2) contain a fragment of 3.5 kb because of the deletion of pV gene. Secondly, the expression of pV in virus infected CRL cells was analyzed by Western blot using anti-pV serum. As seen in FIG. 13D, a 55 kDa protein could be detected in BAV304a infected CRL cells (lane 1). Similar mol wt protein could be detected in uninfected CRL.pV cells (lane 3). No such band could be detected in BAV.dV infected CRL cells (lane 2).

To determine the influence of pV on the formation of BAdV-3 particle, CRL cells or CRL.pV cells were infected with purified BAV.dV (grown in CRL.pV cells) at a MOI of 2. At 48 hrs post infection, the lysates of infected cells were used to purify virions by CsCl gradient centrifugation. As seen in FIG. 13E, deletion of pV predominantly produced population of virus representing mature virions. Moreover, no visible decrease in the production of the mature virus particles could be observed in BAV.dV grown in CRL.pV cells or CRL cells.

### Growth of BAV.dV in CRL Cells

To determine if BAV.dV can produce infectious viral particles in pV negative CRL cells, viral growth characteristics of CsCl purified BAV304a (grown in CRL cells) and BAV.dV (grown in CRL.pV cells) was analyzed. Monolayers of CRL cells in 24 well were infected with BAV304a or BAV.dV at MOI of 2. The infected cells were harvested at different times (0, 6, 12, 24, 36, 48 hrs) post-infection. After freeze-thawing three times, the samples were titrated by TCID₅₀ in CRL.pV cells. As shown in FIG. 13F, BAV304a grew to a titer of ˜10⁸ TCID₅₀/ml at 48 h post-infection of CRL.pV cells. In contrast, there was no detectable increase in the titer of BAV.dV. Similarly, no detectable increase in the titer of BAV.dV could be observed in MDBK cells (data not shown).

### Example 13. Analysis of Protein Expression in BAV.dV Infected Cells

### Materials and Methods—Antibodies

Production and characterization of anti-DBP (Kulshreshtha et al., 2004), which detect a protein of 48 and 102 kDa in BAdV-3 infected cells, respectively, has been described. The anti-pX serum detects a protein of 25 kDa, anti hexon serum detects a protein of 103 kDa and anti-pVII serum detects proteins of 22 and 20 kDa (Paterson, 2010) in BAdV-3 infected cells. Results

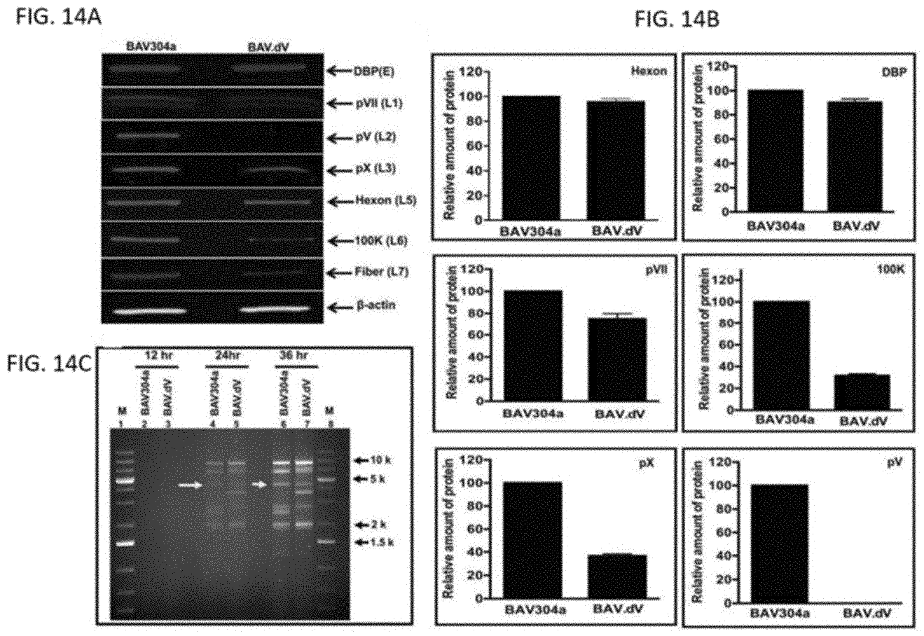

To analyze if deletion of pV modulates the expression of viral proteins, monolayers of CRL cells were infected with BAV304a or BAV.dV at MOI of 2. At 24 hrs post-infection, the cells were harvested and lysed. The proteins from the cell lysates were separated by SDS-PAGE, transferred to nitrocellulose membrane and probed by protein specific anti-serum and secondary antibodies conjugated with fluorophores. Finally, the membranes were scanned and analyzed by Odyssey CLx Imaging System. As expected (FIG. 14A,B), the expression of pV could be detected in BAV304a infected CRL cells but not in BAV.dV infected CRL cells. No appreciable difference could be detected in the expression of early DBP protein in CRL cells infected with BAV304a or BAV.dV. However, compared to BAV304a, reduced expression of some late proteins particularly 100K, pX and pVII were observed in BAV.dV infected cells. Moreover both precursor and cleaved form of pVII could be detected in BAV304a or BAV.dV infected cells.

### Analysis of BAV.dV DNA Replication

The CRL cells were infected with purified BAV304a or BAV.dV (grown in CRL.pV cells) at a MOI of 2. At 12, 24 or 36 hrs post infection, the cells were collected, washed with PBS and used to extract DNA as described (Farina et al., 2001). The DNA isolated from equal number of cells was digested with restriction enzyme Bmt1. Analysis of restriction enzyme digested DNA (FIG. 14C) suggested that both BAV304a and BAV.dV replicated to similar levels in CRL cells.

### Example 14. Analysis of Protein Incorporation in BAV.dV Viral Particles

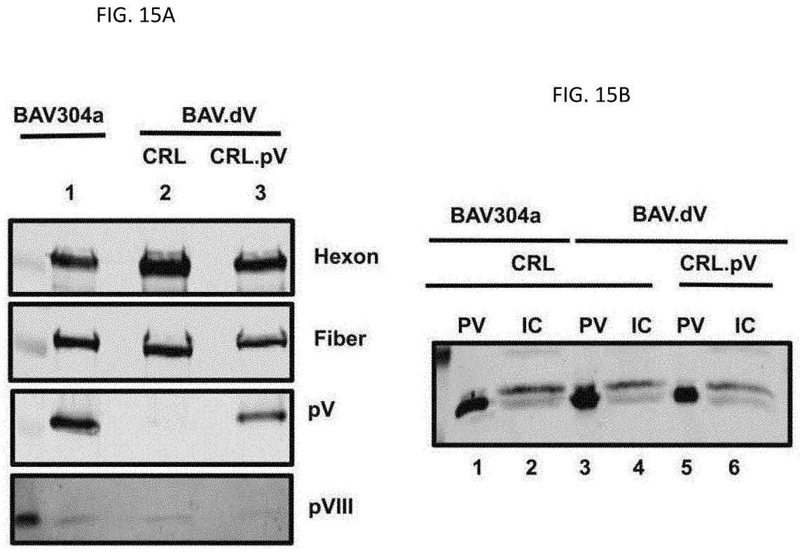

To determine the incorporation of pV in the progeny virions, proteins from purified virions were separated by 10% SDS-PAGE, transferred to nitrocellulose and probed in Western blot using anti-pV serum. As seen in FIG. 15A, anti-pV detected a protein of 55 kDa in purified BAV304a grown in CRL cells (lane 1). Similar protein band could be detected in purified BAV.dV grown in CRL.pV cells (lane 3). However, no such protein could be detected in BAV.dV grown in CRL cells (lane 2). Moreover, there was no detectable difference in the incorporation of the viral proteins in purified BAV304a or BAV.dV virions (grown in CRL cells or CRL.pV cells). As seen in FIG. 15A, hexon and protein were efficiently incorporated in purified BAV304a grown in CRL cells (lane 1), 6 purified BAV.dV grown in CRL cells (lane 2) or purified BAV.dV grown in CRL.pV cells (lane 3).

Anti-pVII serum detected both precursor and cleaved form of pVII in BAV304a infected cells (FIG. 15B, lane 2) or BAV.dV infected CRL cells (FIG. 15B, lane 4) or CRL.pV cells (FIG. 15B, lane 6). As expected, a protein consistent with the cleaved form of pVII could be detected in purified BAV304a grown in CRL cells (FIG. 15B, lane 1), purified BAV.dV grown in CRL cells (FIG. 15B, lane 3) or grown in CRL.pV cells (FIG. 15B, lane 5). Similarly, a cleaved form of pVIII is incorporated in purified BAV304a grown in CRL cells (FIG. 15A, lane 1), BAV.dV grown in CRL cells (FIG. 15A, lane 2) or BAV.dV grown in CRL.pV cells (FIG. 15A, lane 3).

### Example 15. Analysis of BAV.dV by Transmission Electron Microscopy

### Materials and Methods—Transmission Electron Microscopy

CRL cells were infected with BAV304a or BAV.dV at MOI of 2. At 24 hrs post-infection, the cells were collected and fixed in 2.5% glutaraldehyde, and with 1% 0s04 in 0.1M PBS. After dehydration with a graded ethanol series and propylene oxide, the samples were infiltrated with a mixture of propylene oxide and EMbed-812 embedding medium and polymerized in embedding capsules at 60□ C for 24-48 hrs. The pellets were sectioned by using a Reichert ultracut microtome, the sections were stained with 2% uranyl acetate and lead citrate. Finally, the stained sections were viewed using a Philips CM10 TEM.

### Results

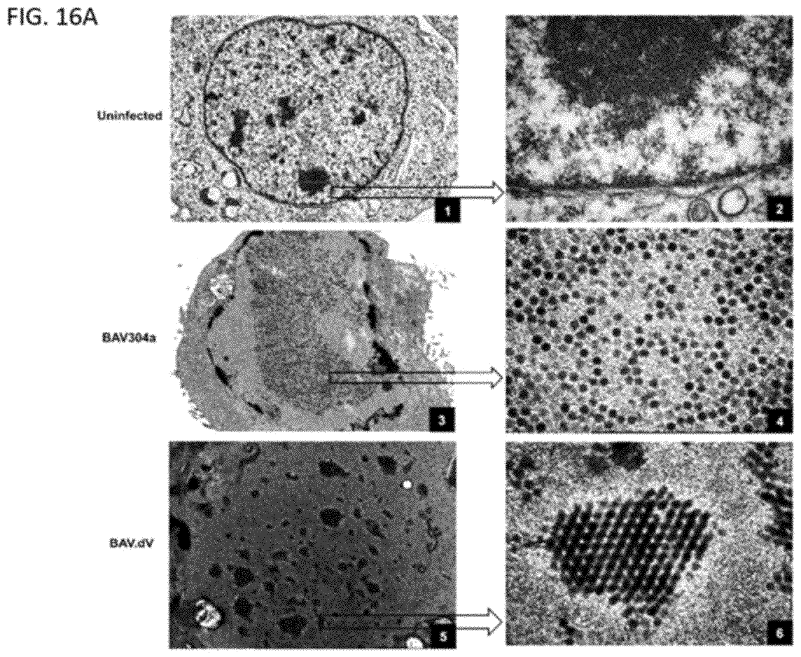

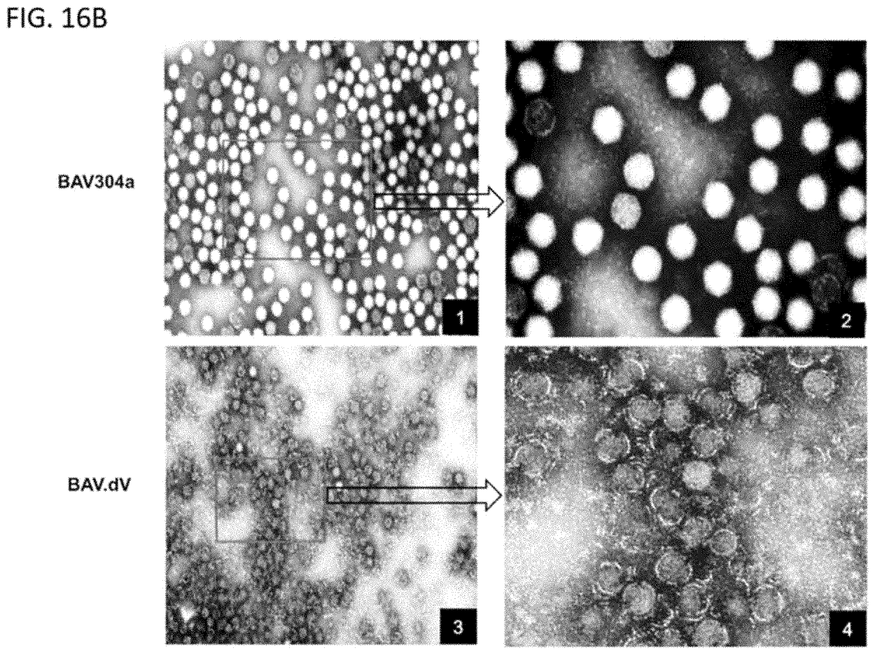

To examine if the deletion of pV affects the formation of BAdV-3 particles, CRL cells were infected with BAV304a or BAV.dV at an MOI of 2. At 24 hrs post infection, the cells were collected, processed and analyzed by TEM. As seen in FIG. 16A, BAV304a (panel 3,4) appeared to produce more viral particles than BAV.dV (panel 5,6) in infected CRL cells. Moreover, BAV304a particles were uniform and loosely arranged (panel 3). In contrast, BAdV.dV particles appeared to be clustered together and appeared tightly organized in rows (panel 5). Analysis of the enlargement of selected areas of TEM images suggested that BAV304a are clearly of typical icosahedral in shape (panel 4). However, BAV.dV showed less clear morphology and did not possess clear icosahedral shape (panel 6). No such virions could be detected in mock infected CRL cells (FIG. 16A, panel 1,2).

Next, we analyzed the CsCl purified BAV304a or BAV.dV (grown in CRL cells) by TEM. The analysis of mature BAV304a virions detected intact capsids with typical icosahedral shape (FIG. 16B, panel 1, 2). In contrast, most of the BAV.dV particles appeared circular in shape with partially degraded capsids (FIG. 16B, panel 3, 4).

### Example 16. Thermostability of BAdV.dV

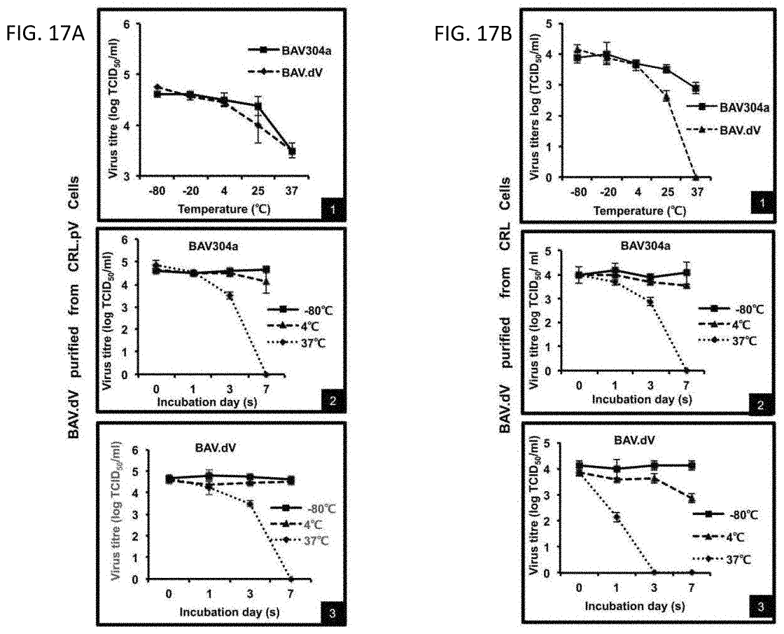

To determine if the deletion of pV alter viral thermostability, purified viral particles in PBS containing 10% glycerol were incubated at different temperatures (−80° C., −20° C., 4° C., 25° C. and 37° C.) for 3 days or incubated at different temperatures (−80° C., 4° C. and 37° C.) for 0, 1, 3 and 7 days. Finally, the infectivity was measured by TCID₅₀ assay. As seen in FIG. 17A, there appeared no difference in the thermostability or dynamics of viral inactivation of BAV304a or BAV.dV grown in CRL.pV cells. In contrast, both thermostability and dynamics of viral inactivation of BAV.dV grown in CRL cells appeared significantly different from BAV304a (FIG. 17B).

### Example 17. Deletion of pV does not Result in Compensatory Mutations

Unlike primary cells (Ugai et al., 2012), HAdV-5 pV is not required for virus replication and formation of infectious virus particles in cancer cells (Ugai et al., 2012). This is due to apparent thermostable mutations (G13E and R17I) in the less conserved region of core protein X/Mu, which compensate for the lack of pV (Ugai et al., 2007). Moreover, analysis of CsCl gradient purified pV deleted HAdV-5 grown in cancer cells show increased incorporation of protein X\Mu in mature virions. In contrast, pV appears essential for the replication of BAdV-3 CRL or MDBK cells. Despite conservation of arginine residue at amino acid 20 of BAdV-3 pV (Ugai et al., 2007), analysis of DNA sequence of different clones of BAV.dV grown (different passages) in CRL or MDBK cells did not reveal any mutation in the core proteins X\Mu or pVII (data not shown). Because of unavailability of reagents, the incorporation of the X\Mu could not be analyzed in CsCl gradient purified BAV.dV grown in CRL cell. Our results suggest that deletion of pV does not introduce compensatory mutations in core proteins X\Mu or pVII.

In summary, we have demonstrated that BAdV-3 pV is essential for the replication of BAdV-3 in CRL (primary) and MDBK (continuous) cells. Analysis of BAV.dV suggested that pV appears to be required for maintaining the integrity of the capsid structure and helps in stability of BAdV-3 capsid. However, lack of pV did not introduce any compensatory mutations in other core proteins x\Mu or pVII. Moreover, pV may have a role in the proteolytic cleavage of pVII.

### REFERENCES

Adachi, Y., Copeland, T. D., Hatanaka, M., and Oroszlan, S. (1993). Nucleolar targeting signal of Rex protein of human T-cell leukemia virus type I specifically binds to nucleolar shuttle protein B-23. The Journal of Biological Chemistry 268, 13930-13934.
Ali, H., LeRoy, G., Bridge, G., and Flint, S. J. (2007). The adenovims L4 33-kilodalton protein binds to intragenic sequences of the major late promoter required for late phasespecific stimulation of transcription. Journal of Virology 81, 1327-1338.
Anand, S K, Gaba, A., Singh, J., and Tikoo, S. K. (2014). Bovine adenovirus 3 core protein precursor pVII localizes to mitochondria, and modulates ATP synthesis, mitochondrial Ca2+ and mitochondrial membrane potential. The Journal of General Virology 95, 442-452.
Anderson, C. W., Baum, P. R., and Gesteland, R. F. (1973). Processing of Adenovirus 2-Induced Proteins. Journal of Virology 12, 241-252.
Ayalew, L. E. (2014). The role of bovine adenovirus (BAdV)-3 protein pVIII in viral replication. PhD Thesis, University of Saskatchewan, Saskatoon.
Ayalew, L. E., Gaba, A., Kumar, P., and Tikoo, S. K. (2014). Conserved regions of bovine adenovirus-3 pVIII contain functional domains involved in nuclear localization and packaging in mature infectious virions. Journal of General Virology 95, 1743-1754.
Bangari, D. S., and Mittal, S. K. (2006). Development of nonhuman adenovimses as vaccine vectors. Vaccine 24, 849-862.
Blanchette, P., Wimmer, P., Dallaire, F., Cheng, C. Y., and Branton, P. E. (2013). Aggresome formation by the adenoviral protein E1B55K is not conserved among adenovims species and is not required for efficient degradation of nuclear substrates. Journal of Virology 87, 4872-4881.
Benko, M., Elo, P., Ursu, K., Ahne, W., LaPatra, S. E., Thomson, D., and Harrach, B. (2002). First molecular evidence for the existence of distinct fish and snake adenovimses. Journal of Virology 76, 10056-10059.
Boulikas, T. (1993). Nuclear localization signals (NLS). Critical Reviews in Eukaryotic Gene Expression 3, 193-227.
Chartier, C., Deglyse, E., Gantzer, M., Dieterle, A., Pavirani, A., and Mehtali, M. (1996). Efficient generation of recombinant adenovims vectors by homologous recombination in Escherichia coli. Journal of Virology 70, 4805-4810.
Chatterjee, P. K., Vayda, M. E., and Flint, S. J. (1985). Interactions among the three adenovirus core proteins. Journal of Virology 55, 379-386.
Cheng, G., Brett, M. E., and He, B. (2002). Signals that dictate nuclear, nucleolar, and cytoplasmic shuttling of the gamma(1)34.5 protein of herpes simplex virus type 1. Journal of Virology 76, 9434-9445.
Cheng, P. H., Rao, X. M., McMasters, K. M., and Zhou, H. S. (2013). Molecular basis for viral selective replication in cancer cells: activation of CDK2 by adenovirus-induced cyclin E. PloS one 8, e57340.
Chiocca, S., Kurzbauer, R., Schaffner, G., Baker, A., Mautner, V., and Cotten, M. (1996). The complete DNA sequence and genomic organization of the avian adenovirus CELO. Journal of Virology 70, 2939-2949.
Cros, J. F., Garcia-Sastre, A., and Palese, P. (2005). An unconventional NLS is critical for the nuclear import of the influenza A virus nucleoprotein and ribonucleoprotein. Traffic (Copenhagen, Denmark) 6, 205-213.
Davison, A. J., Benko, M., and Harrach, B. (2003). Genetic content and evolution of adenoviruses. Journal of General Virology 84, 2895-2908.
Davison, A. J., Wright, K. M., and Harrach, B. (2000). DNA sequence of frog adenovirus. Journal of General Virology 81, 2431-2439.
Du, E., and Tikoo, S. K. (2010). Efficient replication and generation of recombinant bovine adenovirus-3 in nonbovine cotton rat lung cells expressing I-SceI endonuclease. The Journal of Gene Medicine 12, 840-847.
Earley, L. F., Kawano, Y., Adachi, K., Sun, X. X., Dai, M. S., and Nakai, H. (2015). Identification and characterization of nuclear and nucleolar localization signals in the adeno-associated virus serotype 2 assembly-activating protein. Journal of Virology 89, 3038-3048.
Farina, S. F., Gao, G. P., Xiang, Z. Q., Rux, J. J., Burnett, R. M., Alvira, M. R., Marsh, J., Ertl, H. C., and Wilson, J. M. (2001). Replication-defective vector based on a chimpanzee adenovirus. Journal of Virology 75, 11603-11613.
Finnen, R. L., Biddle, J. F., and Flint, J. (2001). Truncation of the human adenovirus type 5 L4 33-kDa protein: evidence for an essential role of the carboxy-terminus in the viral infectious cycle. Virology 289, 388-399.
Gomez Corredor, A., and Archambault, D. (2009). The Bovine Immunodeficiency Virus Rev Protein: Identification of a Novel Lentiviral Bipartite Nuclear Localization Signal Harboring an Atypical Spacer Sequence. Journal of Virology 83, 12842-12853.
Hindley, C. E., Davidson, A. D., and Matthews, D. A. (2007). Relationship between adenovirus DNA replication proteins and nucleolar proteins B23.1 and B23.2. The Journal of General Virology 88, 3244-3248.
Hiscox, J. A. (2007). RNA viruses: hijacking the dynamic nucleolus. Nature Reviews. Microbiology 5, 119-127.
Hong, S. S., Szolajska, E., Schoehn, G., Franqueville, L., Myhre, S., Lindholm, L., Ruigrok, R. W., Boulanger, P., and Chroboczek, J. (2005). The 100K-chaperone protein from adenovirus serotype 2 (Subgroup C) assists in trimerization and nuclear localization of hexons from subgroups C and B adenoviruses. Journal of Molecular Biology 352, 125-138.
Horne, R., Brenner, S., Waterson, A., and Wildy, P. (1959). The icosahedral form of an adenovirus. Journal of Molecular Biology 1, 84-IN15.
Howes, S. H., Bockus, B. J., and Schaffhausen, B. S. (1996). Genetic analysis of polyomavims large T nuclear localization: nuclear localization is required for productive association with pRb family members. Journal of Virology 70, 3581-3588.
Idamakanti, N, Reddy, P. S., Babiuk, L. A., and Tikoo, S. K. (1999). Transcription mapping and characterization of 284R and 121R proteins produced from early region 3 of bovine adenovirus type 3. Virology 256, 351-359.
Kohler, M., Speck, C., Christiansen, M., Bischoff, F R., Prehn, S., Haller, H., Gorlich, D., and Hartmann, E (1999). Evidence for distinct substrate specificities of importin alpha family members in nuclear protein import. Molecular and Cellular Biology 19, 7782-7791.
Kosugi, S., Hasebe, M., Matsumura, N., Takashima, H, Miyamoto-Sato, E., Tomita, M., and Yanagawa, H. (2009). Six classes of nuclear localization signals specific to different binding grooves of importin alpha. The Journal of Biological Chemistry 284, 478-485.
Kovacs, G. M., LaPatra, S. E., D'Halluin, J. C., and Benko, M. (2003). Phylogenetic analysis of the hexon and protease genes of a fish adenovirus isolated from white sturgeon (Acipenser transmontanus) supports the proposal for a new adenovirus genus. Virus Research 98, 27-34.
Kulshreshtha, V., Ayalew, L E., Islam, A., and Tikoo, S. K. (2014). Conserved arginines of bovine adenovirus-3 33K protein are important for transportin-3 mediated transport and virus replication. PloS 1 9, e101216.
Kulshreshtha, V., Babiuk, L. A., and Tikoo, S. K. (2004). Role of bovine adenovirus-3 33K protein in viral replication. Virology 323, 59-69.
Kulshreshtha, V., Islam, A., Ayalew, L. E., and Tikoo, S. K. (2015). Leucine residues in conserved region of 33K protein of bovine adenovirus-3 are important for binding to major late promoter and activation of late gene expression. Virology 483, 174-184.
Ladd, A. N., and Cooper, T. A. (2004). Multiple domains control the subcellular localization and activity of ETR-3, a regulator of nuclear and cytoplasmic RNA processing events. Journal of Cell Science 117, 3519-3529.
Lam, Y. W., Evans, V. C., Heesom, K. J., Lamond, A. I., and Matthews, D. A. (2010). Proteomics analysis of the nucleolus in adenovirus-infected cells. Molecular & Cellular Proteomics: MCP 9, 117-130.
Lehmberg, E., Traina, J. A., Chakel, J. A., Chang, R. J., Parkman, M., McCaman, M T, Murakami, P. K., Lahidji, V., Nelson, J. W., Hancock, W. S., et al. (1999). Reversed-phase high-performance liquid chromatographic assay for the adenovirus type 5 proteome. Journal of Chromatography. B, Biomedical sciences and applications 732, 411-423.
Leong, K., Lee, W., and Berk, A. J. (1990). High-level transcription from the adenovirus major late promoter requires downstream binding sites for late-phase-specific factors. Journal of Virology 64, 51-60.
Li, X., Bangui, D. S., Sharma, A., and Mittal, S. K. (2009). Bovine adenovirus serotype 3 utilizes sialic acid as a cellular receptor for virus entry. Virology 392, 162-168.
Lutz, P., Puvion-Dutilleul, F., Lutz, Y., and Kedinger, C. (1996). Nucleoplasmic and nucleolar distribution of the adenovirus IVa2 gene product. Journal of Virology 70, 3449-3460.
Makadiya, N., Gaba, A., and Tikoo, S. K. (2015). Cleavage of bovine adenovirus type 3 non-structural 100K protein by protease is required for nuclear localization in infected cells but is not essential for virus replication. Journal of General Virology 96, 2749-2763.
Mangel, W. F., and San Martin, C. (2014). Structure, Function and Dynamics in Adenovirus Maturation. Viruses 6, 4536-4570.
Matthews, D. A. (2001). Adenovirus protein V induces redistribution of nucleolin and B23 from nucleolus to cytoplasm. Journal of Virology 75, 1031-1038.
Matthews, D. A., and Russell, W. C. (1998). Adenovirus core protein V interacts with p32—a protein which is associated with both the mitochondria and the nucleus. Journal of General Virology 79, 1677-1685.
Melen, K., Kinnunen, L., Fagerlund, R., Ikonen, N., Twu, K. Y., Krug, R. M., and Julkunen, I. (2007). Nuclear and nucleolar targeting of influenza A virus NS1 protein: striking differences between different virus subtypes. Journal of Virology 81, 5995-6006.
Musinova, Y. R., Kananykhina, E Y, Potashnikova, D. M., Lisitsyna, O. M., and Sheval, E. V. (2015). A charge-dependent mechanism is responsible for the dynamic accumulation of proteins inside nucleoli. Biochimica et Biophysica Acta 1853, 101-110.
Nigg, E. A. (1997). Nucleocytoplasmic transport: signals, mechanisms and regulation. Nature 386, 779-787.
Olson, M. O., and Dundr, M. (2005). The moving parts of the nucleolus. Histochemistry and Cell Biology 123, 203-216.
Papp, Z., Middleton, D. M., Mittal, S. K., Babiuk, L. A., and Baca-Estrada, M. E. (1997). Mucosal immunization with recombinant adenoviruses: induction of immunity and protection of cotton rats against respiratory bovine herpesvirus type 1 infection. Journal of General Virology 78, 2933-2943.
Paterson, C. P. (2010). Molecular characterization of 52K protein of bovine adenovirus type 3. PhD Thesis, University of Saskatchewan, Saskatoon.
Paterson, C. P., Ayalew, L E., and Tikoo, S. K. (2012). Mapping of nuclear import signal and importin alpha3 binding regions of 52K protein of bovine adenovirus-3. Virology 432, 63-72.
Reddy, P. S., Idamakanti, N., Zakhartchouk, A. N., Baxi, M. K., Lee, J. B., Pyne, C., Babiuk, L. A., and Tikoo, S. K. (1998). Nucleotide sequence, genome organization, and transcription map of bovine adenovirus type 3. Journal of Virology 72, 1394-1402.
Reed, M. L., Dove, B. K., Jackson, R. M., Collins, R., Brooks, G., and Hiscox, J. A. (2006). Delineation and modelling of a nucleolar retention signal in the coronavirus nucleocapsid protein. Traffic (Copenhagen, Denmark) 7, 833-848.
Richardson, W. D., Roberts, B. L., and Smith, A. E. (1986). Nuclear location signals in polyoma virus large-T. Cell 44, 77-85.
Salvetti, A., and Greco, A. (2014). Viruses and the nucleolus: the fatal attraction. Biochimica et Biophysica Acta 1842, 840-847.
Scott, M. S., Boisvert, F. M., McDowall, M. D., Lamond, A. I., and Barton, G. J. (2010). Characterization and prediction of protein nucleolar localization sequences. Nucleic Acids Res 38, 7388-7399.
Sheng, Z., Lewis, J. A., and Chirico, W. J. (2004). Nuclear and nucleolar localization of 18-kDa fibroblast growth factor-2 is controlled by C-terminal signals. The Journal of Biological Chemistry 279, 40153-40160.
Stracker, T. H., Lee, D. V., Carson, C. T., Araujo, F. D., Ornelles, D. A., and Weitzman, M D (2005). Serotype-specific reorganization of the Mre 11 complex by adenoviral E4orf3 proteins. Journal of Virology 79, 6664-6673.
Taylor, L. A., and Rose, R. E. (1988). A correction in the nucleotide sequence of the Tn903 kanamycin resistance determinant in pUC4K. Nucleic Acids Res 16, 358.
Thanbichler, M., Iniesta, A. A., and Shapiro, L. (2007). A comprehensive set of plasmids for vanillate- and xylose-inducible gene expression in Caulobacter crescentus. Nucleic Acids Res 35, e 137.
Thompson, K. G., Thomson, G. W., and Henry, J. N. (1981). Alimentary tract manifestations of bovine adenovirus infections. The Canadian Veterinary Journal. 22, 68-71.
Tollefson, A. E., Hermiston, T. W., and Wold, W. S. M. (1999). Preparation and Titration of CsCl-Banded Adenovirus Stock. In Adenovirus Methods and Protocols, W. S. M. Wold, ed. (Totowa, N. J.: Springer New York), pp. 1-9.
Ugai, H., Borovjagin, A. V., Le, L. P., Wang, M., and Curiel, D. T. (2007). Thermostability/infectivity defect caused by deletion of the core protein V gene in human adenovirus type 5 is rescued by thermo-selectable mutations in the core protein X precursor. Journal of Molecular Biology 366, 1142-1160.
Ugai, H., Dobbins, G. C., Wang, M., Le, L. P., Matthews, D. A., and Curiel, D. T. (2012). Adenoviral protein V promotes a process of viral assembly through nucleophosmin 1. Virology 432, 283-295.
Valdez, B. C., Perlaky, L., Henning, D., Saijo, Y., Chan, P. K., and Busch, H. (1994). Identification of the nuclear and nucleolar localization signals of the protein p120. Interaction with translocation protein B23. The Journal of Biological Chemistry 269, 23776-23783.
Vayda, M. E., Rogers, A. E., and Flint, S. J. (1983). The structure of nucleoprotein cores released from adenovirions. Nucleic Acids Res 11, 441-460.
Walton, T. H., Moen, P. T., Jr., Fox, E., and Bodnar, J. W. (1989). Interactions of minute virus of mice and adenovirus with host nucleoli. Journal of Virology 63, 3651-3660.
Weber, J. D., Kuo, M. L., Bothner, B., DiGiammarino, E. L., Kriwacki, R. W., Roussel, M. F., and Sherr, C. J. (2000). Cooperative signals governing ARF-mdm2 interaction and nucleolar localization of the complex. Molecular and Cellular Biology 20, 2517-2528.
Wodrich, H., Cassany, A., D'Angelo, M. A., Guan, T., Nemerow, G., and Gerace, L. (2006). Adenovirus core protein pVII is translocated into the nucleus by multiple import receptor pathways. Journal of Virology 80, 9608-9618.
Wu, Q., and Tikoo, S. K. (2004). Altered tropism of recombinant bovine adenovirus type-3 expressing chimeric fiber. Virus Research 99, 9-15.
Xi, Q., Cuesta, R., and Schneider, R. J. (2005). Regulation of translation by ribosome shunting through phosphotyrosine-dependent coupling of adenovirus protein 100k to viral mRNAs. Journal of Virology 79, 5676-5683.
Xing, L., and Tikoo, S. K. (2006). E1A promoter of bovine adenovirus type 3. The Journal of General Virology 87, 3539-3544.
Xing, L., and Tikoo, S. K. (2007). Bovine adenovirus-3 ElA coding region contain cisacting DNA packaging motifs. Virus Research 130, 315-320.
Xing, L., Zhang, L., Van Kessel, J., and Tikoo, S. K. (2003). Identification of cis-acting sequences required for selective packaging of bovine adenovirus type 3 DNA. The Journal Of General Virology 84, 2947-2956.
Zakhartchouk, A., Connors, W., van Kessel, A., and Tikoo, S. K. (2004). Bovine adenovirus type 3 containing heterologous protein in the C-terminus of minor capsid protein IX. Virology 320, 291-300.
Zhou, Y., Reddy, P. S., Babiuk, L. A., and Tikoo, S. K. (2001). Bovine adenovirus type 3 E1B(small) protein is essential for growth in bovine fibroblast cells. Virology 288, 264-274.

SEQUENCES
Bovine Adenovirus 3 genome nucleotide sequence
(SEQ ID NO: 1)
catcatcaat aatctacagt acactgatgg cagcggtcca actgccaatc atttttgcca 60
cgtcatttat gacgcaacga cggcgagcgt ggcgtgctga cgtaactgtg gggcggagcg 120
cgtcgcggag gcggcggcgc tgggcggggc tgagggcggc gggggcggcg cgcggggcgg 180
cgcgcggggc ggggcgaggg gcggagttcc gcacccgcta cgtcattttc agacattttt 240
tagcaaattt gcgccttttg caagcatttt tctcacattt caggtattta gagggcggat 300
ttttggtgtt cgtacttccg tgtcacatag ttcactgtca atcttcatta cggcttagac 360
aaattttcgg cgtcttttcc gggtttatgt ccccggtcac ctttatgact gtgtgaaaca 420
cacctgccca ttgtttaccc ttggtcagtt ttttcgtctc ctagggtggg aacatcaaga 480
acaaatttgc cgagtaattg tgcacctttt tccgcgttag gactgcgttt cacacgtaga 540
cagacttttt ctcattttct cacactccgt cgtccgcttc agagctctgc gtcttcgctg 600
ccaccatgaa gtacctggtc ctcgttctca acgacggcat gagtcgaatt gaaaaagctc 660
tcctgtgcag cgatggtgag gtggatttag agtgtcatga ggtacttccc ccttctcccg 720
cgcctgtccc cgcttctgtg tcacccgtga ggagtcctcc tcctctgtct ccggtgtttc 780
ctccgtctcc gccagccccg cttgtgaatc cagaggcgag ttcgctgctg cagcagtatc 840
ggagagagct gttagagagg agcctgctcc gaacggccga aggtcagcag cgtgcagtgt 900
gtccatgtga gcggttgccc gtggaagagg atgagtgtct gaatgccgta aatttgctgt 960
ttcctgatcc ctggctaaat gcagctgaaa atgggggtga tatttttaag tctccggcta 1020
tgtctccaga accgtggata gatttgtcta gctacgatag cgatgtagaa gaggtgacta 1080
gtcacttttt tctggattgc cctgaagacc ccagtcggga gtgttcatct tgtgggtttc 1140
atcaggctca aagcggaatt ccaggcatta tgtgcagttt gtgctacatg cgccaaacct 1200
accattgcat ctatagtaag tacattctgt aaaagaacat cttggtgatt tctaggtatt 1260
gtttagggat taactgggtg gagtgatctt aatccggcat aaccaaatac atgttttcac 1320
aggtccagtt tctgaagagg aaatgtgagt catgttgact ttggcgcgca agaggaaatg 1380
tgagtcatgt tgactttggc gcgccctacg gtgactttaa agcaatttga ggatcacttt 1440
tttgttagtc gctataaagt agtcacggag tcttcatgga tcacttaagc gttcttttgg 1500
atttgaagct gcttcgctct atcgtagcgg gggcttcaaa tcgcactgga gtgtggaaga 1560
ggcggctgtg gctgggacgc ctgactcaac tggtccatga tacctgcgta gagaacgaga 1620
gcatatttct caattctctg ccagggaatg aagctttttt aaggttgctt cggagcggct 1680
attttgaagt gtttgacgtg tttgtggtgc ctgagctgca tctggacact ccgggtcgag 1740
tggtcgccgc tcttgctctg ctggtgttca tcctcaacga tttagacgct aattctgctt 1800
cttcaggctt tgattcaggt tttctcgtgg accgtctctg cgtgccgcta tggctgaagg 1860
ccagggcgtt caagatcacc cagagctcca ggagcacttc gcagccttcc tcgtcgcccg 1920
acaagacgac ccagactacc agccagtaga cggggacagc ccaccccggg ctagcctgga 1980
ggaggctgaa cagagcagca ctcgtttcga gcacatcagt taccgagacg tggtggatga 2040
cttcaataga tgccatgatg ttttttatga gaggtacagt tttgaggaca taaagagcta 2100
cgaggctttg cctgaggaca atttggagca gctcatagct atgcatgcta aaatcaagct 2160
gctgcccggt cgggagtatg agttgactca acctttgaac ataacatctt gcgcctatgt 2220
gctcggaaat ggggctacta ttagggtaac aggggaagcc tccccggcta ttagagtggg 2280
ggccatggcc gtgggtccgt gtgtaacagg aatgactggg gtgacttttg tgaattgtag 2340
gtttgagaga gagtcaacaa ttagggggtc cctgatacga gcttcaactc acgtgctgtt 2400
tcatggctgt tattttatgg gaattatggg cacttgtatt gaggtggggg cgggagctta 2460
cattcggggt tgtgagtttg tgggctgtta ccggggaatc tgttctactt ctaacagaga 2520
tattaaggtg aggcagtgca actttgacaa atgcttactg ggtattactt gtaaggggga 2580
ctatcgtctt tcgggaaatg tgtgttctga gactttctgc tttgctcatt tagagggaga 2640
gggtttggtt aaaaacaaca cagtcaagtc ccctagtcgc tggaccagcg agtctggctt 2700
ttccatgata acttgtgcag acggcagggt tacgcctttg ggttccctcc acattgtggg 2760
caaccgttgt aggcgttggc caaccatgca ggggaatgtg tttatcatgt ctaaactgta 2820
tctgggcaac agaataggga ctgtagccct gccccagtgt gctttctaca agtccagcat 2880
ttgtttggag gagagggcga caaacaagct ggtcttggct tgtgcttttg agaataatgt 2940
actggtgtac aaagtgctga gacgggagag tccctcaacc gtgaaaatgt gtgtttgtgg 3000
gacttctcat tatgcaaagc ctttgacact ggcaattatt tcttcagata ttcgggctaa 3060
tcgatacatg tacactgtgg actcaacaga gttcacttct gacgaggatt aaaagtgggc 3120
ggggccaaga ggggtataaa taggtgggga ggttgagggg agccgtagtt tctgtttttc 3180
ccagactggg ggggacaaca tggccgagga agggcgcatt tatgtgcctt atgtaactgc 3240
ccgcctgccc aagtggtcgg gttcggtgca ggataagacg ggctcgaaca tgttgggggg 3300
tgtggtactc cctcctaatt cacaggcgca ccggacggag accgtgggca ctgaggccac 3360
cagagacaac ctgcacgccg agggagcgcg tcgtcctgag gatcagacgc cctacatgat 3420
cttggtggag gactctctgg gaggtttgaa gaggcgaatg gacttgctgg aagaatctaa 3480
tcagcagctg ctggcaactc tcaaccgtct ccgtacagga ctcgctgcct atgtgcaggc 3540
taaccttgtg ggcggccaag ttaacccctt tgtttaaata aaaatacact catacagttt 3600
attatgctgt caataaaatt ctttattttt cctgtgataa taccgtgtcc agcgtgctct 3660
gtcaataagg gtcctatgca tcctgagaag ggcctcatat accatggcat gaatattaag 3720
atacatgggc ataaggccct cagaagggtt gaggtagagc cactgcagac tttcgtgggg 3780
aggtaaggtg ttgtaaataa tccagtcata ctgactgtgc tgggcgtgga aggaaaagat 3840
gtcttttaga agaagggtga ttggcaaagg gaggctctta gtgtaggtat tgataaatct 3900
gttcagttgg gagggatgca ttcgggggct aataaggtgg agtttagcct gaatcttaag 3960
gttggcaatg ttgcccccta ggtctttgcg aggattcatg ttgtgcagta ccacaaaaac 4020
agagtagcct gtgcatttgg ggaatttatc atgaagcttg gaggggaagg catgaaaaaa 4080
ttttgagatg gctttatggc gccccaggtc ttccatgcat tcgtccataa taatagcaat 4140
aggcccggtt ttggctgcct gggcaaacac gttctgaggg tgggcgacat catagttgta 4200
gtccatggtc aggtcttcat aggacatgat cttaaaggca ggttttaggg tgctgctttg 4260
aggaaccaga gttcctgtgg ggccgggggt gtagttccct tcacagattt gggtctccca 4320
agcaagcagt tcttgcgggg gtatcatgtc aacttggggg actataaaaa aaacagtttc 4380
gggaggtggt tgaatgaggc ccgtagacat aaggtttctg aggagctggg attttccaca 4440
accggttggt ccgtagacca ccccaataac gggttgcatg gtaaagttta aagatttgca 4500
tgaaccgtca gggcgcagat atggcatggt ggcattcatg gcatctctta tcgcctgatt 4560
atagtctgag agggcattga gtagggtggc gccccccata gccagtagct cgtccaagga 4620
agaaaagtgt ctaagaggtt tgaggccttc agccatgggc atggactcta agcactgttg 4680
catgagagca catttgtccc aaagctcaga gacgtggtct agtacatctc catccagcat 4740
agctctttgt ttcttgggtt ggggtggctg ttgctgtagg gggcgagacg gtgacggtcg 4800
atggcccgca gggtgcggtc tttccagggc ctgagcgtcc tcgccagggt cgtctcggtg 4860
accgtgaagg gctgctgatg cgtctgtctg ctgaccagcg agcgcctcag gctgagcctg 4920
ctggtgccga acttttcgtc gcctagctgt tcagtggaat aataacaagt caccagaagg 4980
tcgtaggaga gttgtgaggt ggcatggcct ttgctcgaag tttgccagaa ctctcggcgg 5040
cggcagcttg ggcagtagat gtttttaagg gcatatagtt tgggggctaa gaagacagat 5100
tcctggctgt gggcgtctcc gtggcagcgg gggcactggg tctcgcattc cacaagccaa 5160
gtcagctgag ggttggtggg atcaaagacc agaggacggt tattaccttt caggcggtgc 5220
ttgcctcggg tgtccatgag ttcctttccc ctttgggtga gaaacatgct gtccgtgtct 5280
ccgtagacaa atttgagaat ccggtcttct aggggagtgc ctctgtcttc taaatagagg 5340
atgtctgccc attcagagac aaaggctcta gtccacgcga ggacaaatga agctatgtgt 5400
gaggggtatc tgttattaaa tatgagagag gatttttttt gcaaagtatg caggcacagg 5460
gctgagtcat cagcttccag aaaggtgatt ggtttgtaag tgtatgtcac gtgatggttc 5520
tgggggtctc ccagggtata aaagggggcg tcttcgtctg aggagctatt gctagtgggt 5580
gtgcactgac ggtgcttccg cgtggcatcc gtttgctgct tgacgggtga gtaggtgatt 5640
tttagctctg ccatgacaga ggagctcagg ttgtcagttt ccacgaaggc ggtgcttttg 5700
atgtcgtagg tgccgtctga aatgcctcta acatatttgt cttccatttg gtcagaaaag 5760
acagtgactc tgttgtctag cttagtggca aagctgccat acagggcatt ggacagcagt 5820
ttggcaatgc ttctgagagt ttggtttttc tctttatccg ccctttcctt gggcgcaatg 5880
ttaagttgca cgtagtctct agccagacac tcccactggg gaaatactgt ggtgcggggg 5940
tcgttgagaa tttggactct ccagccgcgg ttatgaagcg tgatggcatc caaacaagtt 6000
accacttccc cccgtagtgt ctcgttggtc cagcagaggc gacctccttt tctggagcag 6060
aagggcggta taacgtccaa gaatgcttct gggggtgggt ctgcatcaat ggtgaatatc 6120
gcgggcagta gggtgcgatc aaaatagtca atgggtctgt gcaactgggt taggcggtct 6180
tgccagtttt taattgcaag cgctcgatca aaggggttca aaggttttcc cgctgggaaa 6240
ggatgggtga gggcgctggc atacatgccg cagatgtcat acacatagat ggcttctgtt 6300
aggacgccta tgtaggtagg atagcatcgg ccgccccgaa tactttctct aacgtaatca 6360
tacatttcat tggaaggggc tagtagaaag ttgcccagag agctcctgtt gggacgctgg 6420
gatcggtaga ctacctgtct gaagatggca tgggaattgg agctgatggt gggcctttgg 6480
aggacattga aattgcagtg gggcagcccc actgacgtgt gaacaaagtc caaataagat 6540
gcttggagtt ttttaaccaa ttcggccgta accagcacgt ccatagcaca gtagtccaag 6600
gtgcgttgca caatatcata ggcacctgaa ttctcttgca gccagagact cttattgaga 6660
aggtactcct cgtcgctgga ccagtagtcc ctctgaggaa aagaatctgc gtcggttcgg 6720
taggtaccta acatgtaaaa ttcatttaca gctttgtaag ggcagcagcc tttttccacg 6780
ggtaaagcgt aagcggcagc tgcgttcctg agactcgtgt gcgtgagagc aaaggtatct 6840
cggaccatga acttcacaaa ctgaaattta tagtctgctg aggtgggagt gccttcctcc 6900
cagtctttga agtcttttcg agcagcatgt gtggggttag gcagagcaaa agttaagtca 6960
ttgaaaagaa tcttgccaca acgaggcatg aaatttctac tgactttaaa agcagctgga 7020
ataccttgtt tgttgttaat gacttgtgcg gctagaacaa tctcatcaaa gccgtttatg 7080
ttgtgcccta cgacatagac ttccaagaaa gtcggttgcc ctttgagttc aagcgtacac 7140
agttcctcga aaggaatgtc gctggcatgg acatagccca gtttgaggca gaggttttct 7200
aagcacggat tatctgccag gaactggcgc caaagcaaag tgctggcagc ttcttgaagg 7260
gcatcccgat actgtttaaa caagctgcct actttgtttc tttgcgggtt gaggtagtag 7320
aaggtatttg cttgctttgg ccagcttgac cacttttgct ttttagctat gttaacagcc 7380
tgttcgcata gctgcgcgtc accaaacaaa gtaaacacga gcataaaagg catgagttgc 7440
ttgccaaagc taccgtgcca agtgtatgtt tccacatcat agacgacaaa gaggcgccgg 7500
gtgtcggggt gagcggccca ggggaaaaac tttatttctt cccaccagtc cgaagattgg 7560
gtgtttatgt ggtgaaagta aaagtcccgg cggcgagtgc tgcaggtgtg cgtctgctta 7620
aaatacgaac cgcagtcggc acatcgctgg acctctgcga tggtgtctat gagatagagc 7680
tttctcttgt gaataagaaa gttgaggggg aagggaaggc gcggcctgtc agcgcgggcc 7740
gggatgcttg taattttcag cttccccttg tatgttttgt aaacgcacat atttgcgttg 7800
cagaaccgga cgagcgtgtc ttggaatgaa aggatatttt ctggttttaa atcaaatggg 7860
cagtgctcca agtgcagttc aaaaaggttt cggagactgc tggaaacgtc tgcgtgatac 7920
ttgacttcca gggtggtccc gtcttcagtc tgaccgtgca gccgtagggt actgcgtttg 7980
gcgaccaggg gcccccttgg ggctttcttt aaaggggacg tcgagggccg aggggcggcc 8040
tttgcctttc gggcctgagg ggcggtagct ggaccggatc gttgagttcg ggcatgggtt 8100
gcagctgttg gcgcaggtct gatgcgtgct gcacgactct gcggttgatt ctctgaatct 8160
ccgggtgttg ggtgaatgct actggccccg tcactttgaa cctgaaagag aggtcgacag 8220
agttaataga tgcatcgtta agctccgcct gtctaataat ttcttccacg tcaccgctgt 8280
ggtctcggta agcaatgtct gtcataaacc gttcgatctc ttcctcgtcc agttctccgc 8340
gaccagctcg gtggaccgtg gctgccaagt ccgtgctaat gcgtcgcatg agctgggaaa 8400
aggcattggt tcccggttca ttccacactc tgctgtatat aacagcgcca tcttcgtctc 8460
gggctcgcat gaccacctgg cccaagttta gctccacgtg gcgagcaaag acggggctga 8520
ggcggaggtg gtggtgcaga taattgagag tggtggctat gtgctccacg atgaagaagt 8580
agatgaccca tctgcggatg gtcgactcgt taatgttgcc ctctcgctcc agcatgttta 8640
tggcttcgta aaagtccaca gcgaagttaa aaaactgctc gttgcgggcg gagactgtca 8700
gctcttcttg caggagacga atgacttcgg ctacggcggc gcggacttct tcggcaaagg 8760
agcgcggcgg cacgtcctcc tcctcctctt cttccccctc cagcgggggc atctccagct 8820
ctaccggttc cgggctgggg gacagggaag gcggtgcggg ccgaacgacc cgtcggcgtc 8880
gggtgggcaa ggggagactc tctatgaatc gctgcaccat ctcgccccgg cgtatccgca 8940
tctcctgggt aacggcacgc ccgtgttctc ggggtcggag ctcaaaagct ccgccccgca 9000
gttcggtcag aggccgcgcc gcgggctggg gcaggctgag tgcgtcaata acatgcgcca 9060
ccactctctc cgtagaggcg gctgtttcga accgaagaga ctgagcatcc acgggatcgc 9120
tgaagcgttg cacaaaagct tctaaccagt cgcagtcaca aggtaggctg agcataggtg 9180
aggctcgctc ggtgttgttt ctgtttggcg gcgggtggct gaggagaaaa ttaaagtacg 9240
cgcaccgcag gcgccggatg gttgtcagta tgatgagatc cctgcgaccc gcttgttgga 9300
ttctgatgcg gtttgcaaag ccccaggctt ggtcttggca tcgcccaggt tcatgcactg 9360
ttcttggagg aatctctcta cgggcacgtt gcggcgctgc gggggcaggg tcagccattt 9420
cggtgcgtcc aaacccacgc aatggttgga tgagagccaa gtccgctact acgcgctctg 9480
ctaggacggc ttgctggatc tgccgcagcg tttcatcaaa gttttccaag tcaatgaagc 9540
ggtcgtaggg gcccgcgttt atggtgtagg agcagtttgc catggtggac cagtccacaa 9600
tctgctgatc tacccgcacc gtttctcggt acaccagtcg gctataggct cgcgtctcga 9660
aaacatagtc gttgcaaacg cgcaccacgt attggtagcc gattaggaag tgcggcggcg 9720
ggtataagta gagcggccag ttttgcgtgg ccggctgtct ggcgcccaga ttccgtagca 9780
tgagtgtggg gtatcggtac acgtgacgcg acatccagga gatgcccgcg gccgaaatgg 9840
cggccctggc gtactcccgg gcccggttcc atatattcct gagaggacga aagattccat 9900
ggtgtgcagg gtctgccccg taagacgcgc gcaatctctc gcgctctgca aaaaacatac 9960
agatgaaaca tttttggggc ttttcagatg atgcatcccg ctttacggca aatgaagccc 10020
agatccgcgg cagtggcggg ggttcctgct gcggccgccg gcgcgagcgt tgactcaggc 10080
ggtactaccg cgccccctgg tgtcgagtgc ggcgaggggg aagggttagc tcggctgtac 10140
gcgcacccgg acacacaccc gcgcgtgtgc gtgaagcgcg atgcggcgga ggcgtacgtt 10200
ccccgggaga acttattccg cgaccgcagc ggggaggaac ccgaagggag ccgagaccta 10260
aagtacaagg ccggtcggca gttgcgcgcc ggcatgcccc gaaagcgggt gctgaccgaa 10320
ggggactttg aggtggatga gcgcactggc atcagctcag ccaaagccca catggaggcg 10380
gccgatctag tgcgggctta cgagcaaacg gtgaagcaag aggctaattt tcaaaagtca 10440
tttaataacc acgtgcggac actgatctcc cgcgaggaga ccaccctggg tttgatgcac 10500
ttgtgggact ttgcggaggc atacgcgcag aaccccggca gcaagaccct tacggcccaa 10560
gtctttctca tcgtgcagca cttgcaagat gagggcattt ttggggaagc tttcttaagc 10620
atagcagagc ccgagggacg atggatgcta gatctgctaa acatattgca gtccattgtg 10680
gtgcaagagc gccagctttc gctatctgaa aaggtagccg cggtgaacta ctccgtagtt 10740
accctgggca aacattatgc ccgcaagatc tttaagagcc cctttgtgcc gcttgacaag 10800
gaggtgaaga tcagtacatt ttatatgcgc gcggtgctta aggtcctggg tctaagtcac 10860
gacctgggca tgtacagaaa cgaaaaggtg gagaagctag ctagcatagg caggcgttcg 10920
ggagatgagc gacgcggagc tgctgttcaa cctccgccgc gcactaacca ctggcgattc 10980
tgaagcattc gatgaaggcg gggactttac ctgggctccg ccaactcgcg cgaccgcggc 11040
ggccgctttg ccggggcccg agtttgagag tgaagagacg gacgatgaag tcgacgaatg 11100
agtgatgcgg acccccgtat ctttcagctg gtcagtcggc aagagaccgt agccatggcc 11160
gaagcgcccc gaagcctggg ccccgcccct tccaatccta gtttgcaggc tttattccaa 11220
agccagccca gcgccgagca ggagtggcac ggcgtgctgg agagagtcat ggcccttaac 11280
aaaaatggag actttggctc gcagccccag gcgaaccggt ttggagccat cctcgaagcc 11340
gtggtgcccc cgcgctccga tcccacccat gaaaaagtgc tagctattgt gaatgcgctc 11400
ttggagactc aggccatccg tcgcgatgag gccggacaga tgtacaccgc gctgttgcag 11460
cgggtggcca gatacaacag tgtgaatgtg cagggcaatt tggacaggct gattcaggac 11520
gtgaaggagg ctctggcgca gcgcgagcgc accgggccgg gggccggcct agggtctgtg 11580
gtagccttga atgccttcct gagcacacag ccagcggtgg tggagagggg ccaggagaac 11640
tatgtggcct ttgtgagcgc cttaaaactc atggtgaccg aggcgccgca gtctgaggtt 11700
taccaggccg gacctagttt cttttttcaa accagccggc acggttcgca gacggtaaac 11760
ctcagtcagg cctttgataa cttgcgaccc ctctggggcg tgcgcgcgcc agtacacgag 11820
cgtactacca tctcctctct gctcacacca aacacccgct tgctcttgct cctcattgcg 11880
ccctttacgg acagcgtggg catatcccgg gacagttacc tggggcatct gctgaccctt 11940
taccgggaga ccataggtaa cactcgagtt gatgagacca cgtacaacga gatcacggaa 12000
gtgagtcggg ccctgggcgc cgaagacgcg tctaacttgc aagccactct caactactta 12060
ctcacaaata agcagagcaa gttgccacag gagttttctc tgagtcccga agaggagcgg 12120
gtgctgcgct acgtgcagca atctgtcagt ttatttttaa tgcaggatgg acacacggcc 12180
accactgctc tagatcaggc tgcggccaac atagcgccct cgttttacgc gtcccaccgc 12240
gactttataa accgactgat ggactatttc cagcgagctg cggctatggc ccctgactac 12300
tttttacagg ctgttatgaa tccccactgg ctcccgccgc cgggtttctt tactcaggag 12360
tttgactttc cggagcccaa cgaaggcttc ctgtgggatg atttggacag cgcgctccta 12420
cgcgcgcacg taaaagaaga ggaggatcaa ggagctgtgg gcggcacgcc ggcggcttcg 12480
gcgcccgcgt ctcgcgcgca cacaccaccg ccgccgcccg gtgccgcgga cctctttgct 12540
cctaacgcct tccgcaatgt gcaaaataac ggcgtggatg aacttattga cggcttaagc 12600
agatggaaga cttacgccca ggagaggcag gaagtcgttg agcggcacag gcgcagagag 12660
gcgcgtcgcc gggcgcgcga ggcgcgtcta gagtcgagcg atgatgacga cagcgaccta 12720
gggccgtttc tacggggcac ggggcacctc gttcacaacc agtttatgca tctgaagccc 12780
cggggtcccc gccagttttg gtaaccgcac tgtattaagc tgtaagtcct ctcatttgac 12840
acttaccaaa gccatggtct tgcttcgcct ctgacacttt ctctcccccc acacgcggca 12900
ccctacagcc taggggcgat gctccagccc gaactgcagc caattccgct gtcccgccgc 12960
cggcttatga ggcggtggtg gctggggcct tccagacgct ttctcttcga cgagatccac 13020
gtcccgccgc gatatgctgc cgcgtctgcg gggagaaaca gtatccgtta ttccatgctg 13080
cccccgttgt atgacaccac gaagatatac cttatcgaca acaaatcttc agacatccaa 13140
actctgaatt accaaaacga ccactcagat tacctcacta ccatcgtgca gaacagcgac 13200
ttcacgcccc tggaggctag caaccacagc atcgagctag acgagcggtc ccgctggggc 13260
ggaaacctta aaaccatcct ttatacaaac ctgcctaata tcacccagca catgttttct 13320
aactcttttc gggtaaagat gatggcctca aaaaaagacg gcgtgcccca gtacgagtgg 13380
ttccccctaa ggctgcccga gggtaacttt tctgagacta tggtcattga cctcatgaac 13440
aatgccatcg tagagctgta cttggctttg gggcgccagg agggcgtgaa ggaagaggac 13500
atcggggtaa agatcgatac gcgcaacttt agtctgggct atgacccgca gacccagtta 13560
gtgacgcccg gcgtatacac caatgaagct atgcatgcgg acatcgtgtt gctgccgggc 13620
tgtgctatag actttacgca ctcccgatta aacaacctct tgggcatacg caagcgtttt 13680
ccgtaccaag agggcttcgt catctcctat gaggacctta aggggggtaa catccccgct 13740
ttgatggacg tggaggagtt taacaagagc aagacggttc gagctttgcg ggaggacccc 13800
aaggggcgca gttatcacgt gggcgaagac ccagaagcca gagaaaacga aaccgcctac 13860
cgcagctggt acctggctta caattacggg gacccagaaa aaggggtgcg ggccaccaca 13920
ctgctgacta ccggcgacgt gacctgcggg gtggaacaga tctactggag cttgccggac 13980
atggcactgg acccagtcac tttcaaggct tcgctgaaaa ctagcaatta ccccgtggtg 14040
ggcacagaac ttttgccact ggtgccgcgt agcttttata acgctcaggc tgtgtactca 14100
cagtggatac aagaaaaaac taaccagacc cacgttttca atcgctttcc cgaaaatcag 14160
atcttggtgc ggccccctgc gcctaccatc acgtccataa gtgaaaataa gcccagcttg 14220
acagatcacg gaatcgtgcc gctccggaac cgcttggggg gcgtgcaacg tgtgactttg 14280
actgacgcgc ggcgaagatc ctgcccctac gtctacaaga gcttaggcat tgtgacgccg 14340
caagtgctat ctagccgcac gttttaagca gacaggggca cagcagccgt tttttttttt 14400
tttttttcgc tccaccaggg actgtcagga acatggccat tctaatctct cctagcaata 14460
acacgggctg gggcctggga tgcaataaga tgtacggggg cgctcgcata cgttcagact 14520
tgcatccagt gaaggtgcgg tcgcattatc gggccgcctg gggcagccgc accggtcggg 14580
tgggtcgccg cgcaaccgca gctttagccg atgccgtcgc ggccaccggt gatccggtgg 14640
ccgacacaat cgaggcggtg gtggctgacg cccgccagta ccggcgccgc agacggcgag 14700
gggtgcgccg agtcagaagg ttgcgtcgga gcccccgcac tgccctgcag cgacgggttc 14760
gtagcgtacg ccgacaagtg gcgagggccc gcagggtggg ccggcgcgcg gccgctatcg 14820
cagcagacgc ggccatggcc atggcggcgc cagctcggcg acgccgtaac atctactggg 14880
tacgcgatgc ggcaaccgga gcccgcgttc cggtgacaac ccggcctacg gtcagcaaca 14940
ccgtttgaaa tgtctgctac ttttttttgc ttcaataaaa gcccgccgac tgatcagcca 15000
caccttgtca cgcagaattc tttcaaacca ttgcgctctc agcgcgcgcg ccgataaacc 15060
cactgtgatg gcctcctctc ggttgattaa agaagaaatg ttagacatcg tggcgcctga 15120
gatttacaag cgcaaacggc ccaggcgaga acgcgcagca ccgtatgctg tgaagcagga 15180
ggagaagcct ttagtaaagg cggagcgcaa aattaagcgc ggctccagaa agcgggcctt 15240
gtcaggcgtt gacgttcctc tgcccgatga cggctttgag gacgacgagc cccacataga 15300
atttgtgtct gcgccgcgtc ggccctacca gtggaagggc aggcgggtgc gccgggtttt 15360
gcgtcccggc gtggccgtta gtttcacgcc cggcgcgcgc tccctccgtc cgagttccaa 15420
gcgggtgtat gacgaggtgt acgcagacga cgacttctta gaagcggccg cggcccgtga 15480
gggggagttt gcttacggaa agcggggacg cgaggcggcc caggcccagc tgctaccggc 15540
tgtggccgtg ccggaaccga cttacgtagt tttggatgag agcaacccca ccccgagcta 15600
caagcctgta accgagcaga aagttattct ttcccgcaag cggggtgtgg ggaaggtaga 15660
gcctaccatc caggttttag ctagcaagaa gcggcgcatg gccgagaatg aggatgaccg 15720
cggggccggc tccgtggccg aagtgcagat gcgagaagtt aaaccggtaa ccgctgcctt 15780
gggtattcag accgtggatg ttagcgtgcc cgaccacagc actcccatgg aggtcgtgca 15840
gagtctcagt cgggcggctc aagtagctca acgcctgacc caacaacagg tgcggccttc 15900
ggctaagatt aaagtggagg ccatggatct ttctgctccc gtagacgcaa agcctcttga 15960
cttaaaaccc gtggacgtaa agccgacccc gaccttcgtg cttcccagct ttcgttcact 16020
cagcacccaa actgactctt tgcccgcggc agtggtcgtg ccgcgcaagc cccgcgtgca 16080
ccgtgctact aggcgtactg cgcgcggctt gctgccctat taccgcctgc atcctagcat 16140
cacgccgaca ccgggttacc gaggatctgt ctacacgagc tcgggtgtgc gcctgcccgc 16200
cgtccgggcg ccgccgtcgc cgccgtaccc gcagggcgac tccccgcctc agcgctgccg 16260
cggccgcggc gctgctgccc ggcgtgcgct atcaccctag catccgccaa gcggccacag 16320
taacccggct ccgccgttaa gcgctgtgaa actgcaacaa caacaacaaa aataaaaaaa 16380
agtctccgct ccactgtgca ccgttgtcca tcggctaata aagtcccgct ttgtgcgccg 16440
caggaaccac tatccgtaac ctgcgaaaat gagtccccgc ggaaatctga cttacagact 16500
gagaataccg gtcgccctca gtggccggcg ccggcgccga acaggcttgc gaggagggtc 16560
tgcgtacctg ctcggccgcc gcagaaggcg cgcgggcggc ggccgcctgc gcgggggctt 16620
ccttcccctc ctggctccca tcattgcagc cgccatcggc gcaatccccg gcatcgcatc 16680
agtggccatt caggcggccc acaacaaata gggacagtgt aaagaaagct caatctcaat 16740
aaaacaaacc gctcgatgtg cataacgctc tcggcctgca acttctgctg cttacgtctt 16800
tgaccaaagt cactactgtt ttccttttac ccagagccgg cgccagcccc acacagcttg 16860
ttaacacgcc atggacgaat acaattacgc ggctcttgct ccccggcaag gctcccgacc 16920
catgctgagc cagtggtccg gcatcggcac gcacgaaatg cacggcggac gttttaatct 16980
gggcagtttg tggagcggga tcaggaatgt gggcagcgcg ttaagaactg gggctctcgg 17040
gcctggcaca gcaatgcggg caagcgttgc gcgcccagct gaaaaagacg ggcttgcaag 17100
aaaagatatt gagggcgtta gcgccggtat ccacggagcc gtggatctgg gccgtcagca 17160
gctagagaaa gctattgagc agcgcctaga gcgtcgcccc accgctgccg gtgtggaaga 17220
ccttccgctt cccccgggaa cagtcttaga agctgatcgt ttaccgccct cctacgccga 17280
agcggtggct gagcgcccgc cgccggctga cgttctcctg cccgcatcct caaagccgcc 17340
ggtggcggtg gtgaccttgc ccccgaaaaa gagagtgtct gaagagcctg tggaggaagt 17400
tgtgattcgt tcctccgcac cgccgtcgta cgacgaggtt atggcaccgc agccgactct 17460
ggtagccgag cagggcgcca tgaaagcagt gcccgtgatt aagccggctc aaccttttac 17520
cccagctgtg cacgaaacgc aacgcatagt gaccaacttg ccaatcacca cagctgtgac 17580
acggcgacgc gggtggcagg gcactctgaa tgacatcgtg ggcctcggcg ttcgtaccgt 17640
gaagcgccgg cggtgctatt gagggggcgc gcagcggtaa taaagagaac ataaaaaagc 17700
aggattgtgt tttttgttta gcggccactg actctccctc tgtgtgacac gtcctccgcc 17760
agagcgtgat tgattgaccg agatggctac cccgtcgatg ctgccgcaat ggtcctactg 17820
cacatcgccg gtcaggacgc gtccgagtac ctgtcccccg gcttggtgca attcgcacaa 17880
gccaccgaat cctactttaa cattgggaac aagtttagaa accccaccgt cgccccgacg 17940
cacgatgtca ccacggagcg ttcgcagcgt ctgcagctcc gcttcgtgcc cgtagaccgg 18000
gaggacacac agtactccta caaaacccgc ttccagctag ccgtgggcga caaccgggtg 18060
ctggacatgg ccagcacgta ttttgacatc cgcggtacgc tggagagggg cgccagtttc 18120
aagccttaca gcggcacggc ctacaactcc tttgccccca acagtgcccc taacaatacg 18180
cagtttaggc aggccaacaa cggtcatcct gctcagacca tagctcaagc ttcttacgtg 18240
gctaccatcg gcggtgccaa caatgacttg caaatgggtg tggacgagcg tcagcagccg 18300
gtgtatgcga acactacgta ccagccggaa cctcagctcg gcattgaagg ttggacagct 18360
ggatccatgg cggtcatcga tcaagcaggc gggcgggttc tcaggaaccc tactcaaact 18420
ccctgctacg ggtcctatgc taagccgact aacgagcacg ggggcattac taaagcaaac 18480
actcaggtgg agaaaaagta ctacagaaca ggggacaacg gtaacccgga aacagtgttt 18540
tatactgaag aggctgacgt gctaacgccc gacacccacc ttgttcacgc ggtaccggcc 18600
gcggatcggg caaaggtgga ggggctatct cagcacgcag ctcccaacag gccgaacttt 18660
atcggctttc gggactgctt tgtaggcttg atgtattata acagcggggg caacctgggc 18720
gtcttagcgg gtcaatcctc tcagctgaat gccgtggtag acctgcaaga ccgcaacact 18780
gagctttcct atcagatgct tcttgcaaac acgacggaca gatcccgcta ttttagcatg 18840
tggaaccaag ccatggactc gtacgacccg gaggtcaggg tgatagataa cgtgggcgta 18900
gaggacgaga tgcctaatta ctgctttccg ttgtcggggg ttcagattgg aaaccgtagc 18960
cacgaggttc aaagaaacca acaacagtgg caaaatgtag ctaatagtga caacaattac 19020
ataggcaagg ggaacctacc ggccatggag ataaatctag cggccaatct ctggcgttcc 19080
tttttgtaca gtaatgtggc gttgtacttg ccagacaacc ttaaattcac ccctcacaac 19140
attcaactcc cgcctaacac gaacacctac gagtacatga acgggcgaat ccccgttagc 19200
ggccttattg atacgtacgt aaatataggc acgcggtggt cgcccgatgt gatggacaac 19260
gtgaatccct ttaaccacca ccgcaactcg ggcctgcgtt accgctccca gctgctgggc 19320
aacggccgct tctgcgactt tcacattcag gtgccacaaa agttttttgc tattcgaaac 19380
ctgcttctcc tgcccggcac gtacacttac gagtggtcct ttagaaagga cgtaaacatg 19440
atccttcaga gcactctggg caatgatctg cgggtcgatg gggccactgt taatattacc 19500
agcgtcaacc tctacgccag cttctttccc atgtcacata acaccgcttc cactttggaa 19560
gctatgctcc gcaacgacac taatgaccag tcttttaatg actatctctc ggcggctaac 19620
atgttgtatc ccattccgcc caatgccacc caactgccca tcccctcacg caactgggca 19680
gcgttccgtg gctggagtct cacccggcta aaacagaggg agacaccggc gctggggtcc 19740
ccgttcgatc cctatttcac ctattcgggc accatcccgt acctggacgg cactttttac 19800
ctcagccaca cctttcgcaa ggtggccatc cagtttgact cttctgtgac ctggcccggc 19860
aatgacaggc ttttaacccc taacgagttc gaaataaaaa taagtgtgga cggtgaaggc 19920
tacaacgtgg ctcagagcaa tatgactaag gactggttcc tggtgcagat gctagcgaat 19980
tacaacatag gctaccaggg atatcacctg cccccggact acaaggacag gacattttcc 20040
ttcctgcata acttcatacc catgtgccga caggttccca acccagcaac cgagggctac 20100
tttggactag gcatagtgaa ccatagaaca actccggctt attggtttcg attctgccgc 20160
gctccgcgcg agggccaccc ctacccccaa ctggccttac cccctcattg ggacccacgc 20220
catgccctcc gtgacccaga gagaaagttt ctctgcgacc gcaccctctg gcgaatcccc 20280
ttctcctcga acttcatgtc catggggtcc ctcacagatc tcggacagaa cctactgtat 20340
gccaatgccg cgcatgccct agacatgact tttgagatgg atcccatcaa tgagcccact 20400
ctgctgtacg ttctgtttga ggtgtttgac gtggcccgcg ttcaccagcc ccacagaggc 20460
gtgatcgaag tggtgtactt gagaacgcca ttctcagccg gcaacgctac cacataagtg 20520
ccggcttccc tctcaggccc cgcgatgggt tctcgggaag aggagctgag attcatcctt 20580
cacgatctcg gtgtggggcc atacttcctc ggcactttcg ataaacactt tccggggttc 20640
atctccaaag accgaatgag ctgtgccata gtcaacactg ccggacgcga aaccgggggc 20700
gtgcattggc tggccatggc ttggcaccca gcctcgcaga ccttttacat gtttgaccct 20760
ttcggtttct cggatcaaaa gctaaagcaa atttacaact ttgagtatca gggcctccta 20820
aagcgcagcg ccctgacttc cactgctgac cgctgcctga cccttattca aagcactcaa 20880
tctgtccagg gacccaacag cgccgcctgc ggtctgttct gctgcatgtt cctccacgcc 20940
tttgtccgct ggccgcttag ggccatggac aacaatccca ccatgaacct catccacgga 21000
gttcccaaca acatgttgga gagccccagc tcccaaaatg tgtttttgag aaaccagcaa 21060
aatctgtacc gtttcctaag acgccactcc ccccattttg ttaagcatgc ggctcaaatt 21120
gaggctgaca ccgcctttga taaaatgtta acaaattaga ccgtgagcca tgattgcaga 21180
agcatgtcat ttttttttta ttgtttaaaa taaaaacaac acataacatc tgccgcctgt 21240
cctcccgtga tttcttctgc tttatttgca aatggggggc accttaaaac aaagagtcat 21300
ctgcatcgta ctgatcgatg ggcagaataa cattctgatg ctggtactgc gggtcccagc 21360
ggaattcggg aatggtaatg ggggggctct gtttaaccag cgcggaccac atctgcttaa 21420
ccagctgcaa ggctgaaatc atatctggag ccgaaatctt gaaatcgcag tttcgctggg 21480
cattagcccg cgtctgccgg tacacagggt tacagcactg aaatactaac accgatgggt 21540
gttctacgct ggccaggagt ttgggatctt ctacgaggct cttatctacc gcagagcccg 21600
cgttgatatt aaagggcgtt atcttgcata cctgacggcc taggaggggc aattgggagt 21660
gaccccagtt acaatcacac tttaaaggca taagcagatg agttccggca ctttgcatcc 21720
tggggtaaca ggctttctga aaggtcatga tctgccagaa agcctgcaaa gccttgggcc 21780
cctcgctgaa aaacatacca caagactttg aggtaaagct gccggccggc aaagcggcgt 21840
caaagtgaca gcaagccgcg tcttcattct ttagctgcac tacgttcata ttccaccggt 21900
tggtggtgat ctttgtctta tgcggggtct cttttaaagc ccgctgccca ttttcgctgt 21960
tcacatccat ctctatcact tggtctttgg taagcatagg caggccatgc aggcagtgaa 22020
gggccccgtc tcccccctcg gtacactggt ggcgccagac cacacagccc gtggggctcc 22080
acgaggtcgt ccccaggcct gcgactttta acacaaaatc atacaagaag cggcccataa 22140
tagttagcac ggttttctga gtactgaaag taagaggcag gtacacttta gactcattaa 22200
gccaagcttg tgcaaccttc ctaaaacact cgagcgtgcc agtgtcgggc agcaaggtta 22260
agtttttaat atccactttc aaaggcacac acagccccac tgctaattcc atggcccgct 22320
gccaagcaac ttcgtcggct tccagcaagg cccggctggc cgccggcagg gcgggagcgg 22380
cggcctcagc ggctggggct gaaggtttga aaatcttggc gcgcttaacg gctgtgacat 22440
cttcggcggg gggctcagcg atcggcgcgc gccgtttgcg gctgactttt ttccggggcg 22500
tctcatctat cactaagggg ttctcgtccc cgctgctgtc agccgaactc gtggctcgcg 22560
ttaagtcacc gctgcgattc attattctct cctagataac gacaacaaat ggcagagaaa 22620
ggcagtgaaa atcagcggcc agagaacgac actgagctag cagcggtttc agaagcccta 22680
ggcgcggccg cttcggcccc ctcacgtaac tccccgactg acacggattc aggggtggaa 22740
atgacgccca ccagcagccc cgagccgccc gccgctcccc caagttcgcc tgccgcagca 22800
cctgcccctc agaagaacca ggaggagctc tcttcccccg agcccgcggt agcagcagcg 22860
gagccagaag ccgcttcgcg gcccagacca cccacaccca ccgttcaggt cccgcgggag 22920
ccgagcgagg atcaacctga cggacccgcg acgaggcctt cgtacgtgag cgaggattgc 22980
ctcatccgcc atatctctcg ccaggctaac attgttagag acagcctggc agaccgctgg 23040
gagttagagc ccaccgtgtc ggctctctcc gaggcttacg aaaagctcct cttttgtccc 23100
aaggtaccac ccaagaagca agagaatggc acttgcgaac ctgaacctcg cgttaatttt 23160
ttccccacct ttgtagtgcc cgaaacttta gccacgtacc acatcttttt ccaaaaccaa 23220
aaaatccccc tgtcttgtcg cgccaaccgc acccacacag acaccatcat gcacctctac 23280
tcgggggact ccttaccgtg cttccccacg ctgcagctgg tcaacaaaat ctttgaaggc 23340
ttgggctcag aggagcggcg cgcagccaac tcgctgaaag atcaagagga taacagcgcg 23400
ttagttgagc tcgaagggga cagtccccga ctggctgtgg ttaagcgcac actgtctttg 23460
acacatttcg cctaccctgc cataacacta ccgcctaagg tgatggcagc tgtcactggc 23520
agcctcattc atgaatcagc agcgaccgcc gaaccggaag ctgaggcgct gccagaagcc 23580
gaggagcccg tggttagtga ccctgaactt gctcgctggt tggggctcaa cttacaacag 23640
gagcccgagg ccacggccca ggctttggaa gaaagacgca agattatgtt ggcagtatgc 23700
ttagtcacac ttcagctcga gtgcctgcac aagttttttt cttcagagga tgtcatcaaa 23760
aagctgggag agagcctcca ctacgccttt cgccacggct acgtgcgcca agcctgctcc 23820
atttctaacg tggaactaac gaacatcgtc tcatacctgg gtatcttgca cgaaaaccgc 23880
ttgggacaga gtaccctaca cgccaccctt aaagacgaga accgcagaga ctacatcaga 23940
gacacagtct ttctctttct ggtttatact tggcagactg ccatgggcat ttggcagcag 24000
tgcctcgaga ctgagaacgt aaaagaactt gaaaagctct tgcaaaaaag caagagggct 24060
ctctggacgg gcttcgacga gctcaccata gctcaagacc tagctgacat agtgttcccc 24120
cccaaattct tgcacacctt gcaagccggc ctgccagacc ttacatccca gagtctcctt 24180
cacaactttc gctccttcat tttcgaacgc tcgggcattc tacccgccat gtgcaatgca 24240
ctgcccaccg acttcatccc tatcagctac cgggagtgcc ctccaacttt ctgggcctac 24300
acctacctct ttaaactggc caattacctc atgtttcact ccgacatcgc ttacgatcgg 24360
agcggccccg gtctcatgga atgctactgt cgctgcaacc tgtgcagtcc tcaccgctgc 24420
ttggcgacca accccgccct gctcagcgag acccaagtta tcggtacctt cgagattcag 24480
ggccctcctg ctcaagacgg acagccgacc aaaccgcccc tcaggctgac tgcaggtctc 24540
tggacttccg cctacctgcg caaatttgta ccgcaagact tcaacgccca caaaatagcc 24600
ttctacgaag accaatccaa aaagccgaaa gtgaccccca gcgcttgtgt catcactgaa 24660
gaaaaagttt tagcccaatt gcatgaaatt aaaaaagcgc gggaagactt tcctcttaaa 24720
aaggggcacg gagtgtatct ggaccctcag accggcgagg agctgaacgg acccgcaccc 24780
tccgcagcta ggaatgaaac cccgcagcat gtcggcagcc gggccttccg cggctcaggc 24840
ttcggagggc caacagctgc cgccacagac agcggggctg cagccgagca agagggctgt 24900
gaggaaggta gtagcttctc tgaatcccac cgccgccctg gaagacatat ccgaggggga 24960
ggaaggcttc cccctgacgg acgaggaaga cggggacacc ctggagagcg atttcagcga 25020
cttcacggac gaagacgtcg aggaggagga tatgatttcg ataccccgcg accaggggca 25080
ctccggcgag ctcgaggagg gcgaaattcc cgcaacggta gcggcgacgg cggtcaagaa 25140
gggccagggc aagaagagta ggtgggacca gcaggtccgc tccacagcgc ctctaaaggg 25200
cgctagaggt aagaggagct acagctcctg gaaacccctc aagcccacta tcctttcatg 25260
cttactgcag agctccggca gcactgcctt cactcgccgc tatctgcttt ttcgccatgg 25320
cgtgtccgtt ccctccaggg taattcatta ctataattct tactgcagac ccgaagctga 25380
ccaaaaccgc cactcagagc aaaaagagcc gccggagtgc cagcgcggcg cgccctcgcc 25440
ctcctcctct tcctcccaag cgtgctcggg cgccccgccg ccccaaaggc cagcgccatc 25500
aggccgacga cgcaagcacc gagggccgcg acaagcttcg ggagctgatc tttcccactc 25560
tctatgccat attccaacaa agtcgcgctc agcggtgtca cctcaaagtg aaaaatagat 25620
ccttacgttc actgacgcgc agctgcctct accacaacaa ggaggaacag ctccagcgaa 25680
ccctagcaga ctccgaggcg cttctcagta aatactgctc tgcagctccg acacgattct 25740
cgccgccctc ttataccgag tctcccgcca aggacgaatc cggacccgcc taaactctca 25800
gcatgagcaa agaaattccc acaccttatg tttggacctt tcaacctcag atgggagcgg 25860
ccgcaggtgc cagtcaagat tactcgaccc gcatgaattg gttcagcgcg ggacctgata 25920
tgatccacga cgttaacaac attcgtgacg cccaaaaccg catccttatg actcagtcgg 25980
ccattaccgc cactcccagg aatctgattg atcccagaca gtgggccgcc cacctcatca 26040
aacaacccgt ggtgggcacc acccacgtgg aaatgcctcg caacgaagtc ctagaacaac 26100
atctgacctc acatggcgct caaatcgcgg gcggaggcgc tgcgggcgat tactttaaaa 26160
gccccacttc agctcgaacc cttatcccgc tcaccgcctc ctgcttaaga ccagatggag 26220
tctttcaact aggaggaggc tcgcgttcat ctttcaaccc cctgcaaaca gattttgcct 26280
tccacgccct gccctccaga ccgcgccacg ggggcatagg atccaggcag tttgtagagg 26340
aatttgtgcc cgccgtctac ctcaacccct actcgggacc gccggactct tatccggacc 26400
agtttatacg ccactacaac gtgtacagca actctgtgag cggttatagc tgagattgta 26460
agactctcct atctgtctct gtgctgcttt tccgcttcaa gccccacaag catgaagggg 26520
tttctgctca tcttcagcct gcttgtgcat tgtcccctaa ttcatgttgg gaccattagc 26580
ttctatgctg caaggcccgg gtctgagcct aacgcgactt atgtttgtga ctatggaagc 26640
gagtcagatt acaaccccac cacggttctg tggttggctc gagagaccga tggctcctgg 26700
atctctgttc ttttccgtca caacggctcc tcaactgcag cccccggggt cgtcgcgcac 26760
tttactgacc acaacagcag cattgtggtg ccccagtatt acctcctcaa caactcactc 26820
tctaagctct gctgctcata ccggcacaac gagcgttctc agtttacctg caaacaagct 26880
gacgtcccta cctgtcacga gcccggcaag ccgctcaccc tccgcgtctc ccccgcgctg 26940
ggaactgccc accaagcagt cacttggttt tttcaaaatg tacccatagc tactgtttac 27000
cgaccttggg gcaatgtaac ttggttttgt cctcccttca tgtgtacctt taatgtcagc 27060
ctgaactccc tacttattta caacttttct gacaaaaccg gggggcaata cacagctctc 27120
atgcactccg gacctgcttc cctctttcag ctctttaagc caacgacttg tgtcaccaag 27180
gtggaggacc cgccgtatgc caacgacccg gcctcgcctg tgtggcgccc actgcttttt 27240
gccttcgtcc tctgcaccgg ctgcgcggtg ttgttaaccg ccttcggtcc atcgattcta 27300
tccggtaccc gaaagcttat ctcagcccgc ttttggagtc ccgagcccta taccaccctc 27360
cactaacagt ccccccatgg agccagacgg agttcatgcc gagcagcagt ttatcctcaa 27420
tcagatttcc tgcgccaaca ctgccctcca gcgtcaaagg gaggaactag cttcccttgt 27480
catgttgcat gcctgtaagc gtggcctctt ttgtccagtc aaaacttaca agctcagcct 27540
caacgcctcg gccagcgagc acagcctgca ctttgaaaaa agtccctccc gattcaccct 27600
ggtcaacact cacgccggag cttctgtgcg agtggcccta caccaccagg gagcttccgg 27660
cagcatccgc tgttcctgtt cccacgccga gtgcctcccc gtcctcctca agaccctctg 27720
tgcctttaac tttttagatt agctgaaagc aaatataaaa tggtgtgctt accgtaattc 27780
tgttttgact tgtgtgcttg atttctcccc ctgcgccgta atccagtgcc cctcttcaaa 27840
actctcgtac cctatgcgat tcgcataggc atattttcta aaagctctga agtcaacatc 27900
actctcaaac acttctccgt tgtaggttac tttcatctac agataaagtc atccaccggt 27960
taacatcatg aagagaagtg tgccccagga ctttaatctt gtgtatccgt acaaggctaa 28020
gaggcccaac atcatgccgc ccttttttga ccgcaatggc tttgttgaaa accaagaagc 28080
cacgctagcc atgcttgtgg aaaagccgct cacgttcgac aaggaaggtg cgctgaccct 28140
gggcgtcgga cgcggcatcc gcattaaccc cgcggggctt ctggagacaa acgacctcgc 28200
gtccgctgtc ttcccaccgc tggcctccga tgaggccggc aacgtcacgc tcaacatgtc 28260
tgacgggcta tatactaagg acaacaagct agctgtcaaa gtaggtcccg ggctgtccct 28320
cgactccaat aatgctctcc aggtccacac aggcgacggg ctcacggtaa ccgatgacaa 28380
ggtgtctcta aatacccaag ctcccctctc gaccaccagc gcgggcctct ccctacttct 28440
gggtcccagc ctccacttag gtgaggagga acgactaaca gtaaacaccg gagcgggcct 28500
ccaaattagc aataacgctc tggccgtaaa agtaggttca ggtatcaccg tagatgctca 28560
aaaccagctc gctgcatccc tgggggacgg tctagaaagc agagataata aaactgtcgt 28620
taaggctggg cccggactta caataactaa tcaagctctt actgttgcta ccgggaacgg 28680
ccttcaggtc aacccggaag ggcaactgca gctaaacatt actgccggtc agggcctcaa 28740
ctttgcaaac aacagcctcg ccgtggagct gggctcgggc ctgcattttc cccctggcca 28800
aaaccaagta agcctttatc ccggagatgg aatagacatc cgagataata gggtgactgt 28860
gcccgctggg ccaggcctga gaatgctcaa ccaccaactt gccgtagctt ccggagacgg 28920
tttagaagtc cacagcgaca ccctccggtt aaagctctcc cacggcctga catttgaaaa 28980
tggcgccgta cgagcaaaac taggaccagg acttggcaca gacgactctg gtcggtccgt 29040
ggttcgcaca ggtcgaggac ttagagttgc aaacggccaa gtccagatct tcagcggaag 29100
aggcaccgcc atcggcactg atagcagcct cactctcaac atccgggcgc ccctacaatt 29160
ttctggaccc gccttgactg ctagtttgca aggcagtggt ccgattactt acaacagcaa 29220
caatggcact ttcggtctct ctataggccc cggaatgtgg gtagaccaaa acagacttca 29280
ggtaaaccca ggcgctggtt tagtcttcca aggaaacaac cttgtcccaa accttgcgga 29340
tccgctggct atttccgaca gcaaaattag tctcagtctc ggtcccggcc tgacccaagc 29400
ttccaacgcc ctgactttaa gtttaggaaa cgggcttgaa ttctccaatc aagccgttgc 29460
tataaaagcg ggccggggct tacgctttga gtcttcctca caagctttag agagcagcct 29520
cacagtcgga aatggcttaa cgcttaccga tactgtgatc cgccccaacc taggggacgg 29580
cctagaggtc agagacaata aaatcattgt taagctgggc gcgaatcttc gttttgaaaa 29640
cggagccgta accgccggca ccgttaaccc ttctgcgccc gaggcaccac caactctcac 29700
tgcagaacca cccctccgag cctccaactc ccatcttcaa ctgtccctat cggagggctt 29760
ggttgtgcat aacaacgccc ttgctctcca actgggagac ggcatggaag taaatcagca 29820
cggacttact ttaagagtag gctcgggttt gcaaatgcgt gacggcattt taacagttac 29880
acccagcggc actcctattg agcccagact gactgcccca ctgactcaga cagagaatgg 29940
aatcgggctc gctctcggcg ccggcttgga attagacgag agcgcgctcc aagtaaaagt 30000
tgggcccggc atgcgcctga accctgtaga aaagtatgta accctgctcc tgggtcctgg 30060
ccttagtttt gggcagccgg ccaacaggac aaattatgat gtgcgcgttt ctgtggagcc 30120
ccccatggtt ttcggacagc gtggtcagct cacattttta gtgggtcacg gactacacat 30180
tcaaaattcc aaacttcagc tcaatttggg acaaggcctc agaactgacc ccgtcaccaa 30240
ccagctggaa gtgcccctcg gtcaaggttt ggaaattgca gacgaatccc aggttagggt 30300
taaattgggc gatggcctgc agtttgattc acaagctcgc atcactaccg ctcctaacat 30360
ggtcactgaa actctgtgga ccggaacagg cagtaatgct aatgttacat ggcggggcta 30420
cactgccccc ggcagcaaac tctttttgag tctcactcgg ttcagcactg gtctagtttt 30480
aggaaacatg actattgaca gcaatgcatc ctttgggcaa tacattaacg cgggacacga 30540
acagatcgaa tgctttatat tgttggacaa tcagggtaac ctaaaagaag gatctaactt 30600
gcaaggcact tgggaagtga agaacaaccc ctctgcttcc aaagctgctt ttttgccttc 30660
caccgcccta taccccatcc tcaacgaaag ccgagggagt cttcctggaa aaaatcttgt 30720
gggcatgcaa gccatactgg gaggcggggg cacttgcact gtgatagcca ccctcaatgg 30780
cagacgcagc aacaactatc ccgcgggcca gtccataatt ttcgtgtggc aagaattcaa 30840
caccatagcc cgccaacctc tgaaccactc tacacttact ttttcttact ggacttaaat 30900
aagttggaaa taaagagtta aactgaatgt ttaagtgcaa cagactttta ttggttttgg 30960
ctcacaacaa attacaacag catagacaag tcataccggt caaacaacac aggctctcga 31020
aaacgggcta accgctccaa gaatctgtca cgcagacgag caagtcctaa atgttttttc 31080
actctcttcg gggccaagtt cagcatgtat cggattttct gcttacacct ttttagacag 31140
cagtttacac tcatttccgt taaaggatta caactgcggc atatgagaat taagtatata 31200
caactattgc cctttaccca caaacactcc ccccacgggg tgcacctgat gtagctgccc 31260
tcctcaatca tgaaagtgct attaaagtaa attaaatgaa cattattcac atacacgctt 31320
cccacatagg ccaaaaaaac agaggacaac tttgacagct cccgcctgaa ataccaatac 31380
actctatcaa actgcgcacc gtgcacgcac tgctttacca ggccttgaaa gtaaacagcg 31440
gcggaccgac actgcaagct tctaggcttt gggcagtggc agtgaatata tagccactcc 31500
tccccatgca cgtagtagga acgccgcttc ccgggaatca caaatgacaa gcagtagtca 31560
cagaggcaac tagtcaagtg agcgtcctcc tgaggcatga ttaccttcca tggaatgggc 31620
cagtgaatca tagtggcaaa gccagctgca tctggagcgc tgcgaacctt ggctacatgt 31680
ggtgattggc gacgcagatg gagacaggac cttgcattct gaagaccact gcaacagctt 31740
ctgcgtacgc ttgtatttac agtacataaa aaagcacttt tgccacagag cggtcttact 31800
caaccgacag cttttttctt tctgacgctg ccttctgcta ctcaggtagt acaagtccaa 31860
aagagccaaa cggacactca aatccgggtt atctcgatgc tgaagccaga gtccaaaagt 31920
aaccacgcta aaagcctgca tccatatttt gtaactgctg taactccatc ccagagccgg 31980
gcaccgcact tggtccacca tagctgcaaa caaacgggac aattaaggaa agtaaaatga 32040
gcgctggggg cggactcttc tcccgttcgt aggaaacagc cacgtatcaa acaccctttt 32100
caacactggc tctccagccg ctactcgttg aattaatttg tccctgtgct caaacaaccc 32160
acactggtaa cggtggtcgc taggcaaaca tgtcaaatag cacataatca tttccttcac 32220
tttaagcaaa catcgactag cagacacttc acttaattca gcacagtcat agcaaggaat 32280
gattatacac ttgtcatcta atccactgcc catgtacaca ttgccccagg caaaagtggg 32340
cagggacttt aagagctgat tgctcgcccc gacatagttg gtaaaataca gcaaatgcac 32400
cttgttaaca tacacactcc ccacatagta aatataccga gtagacagct tagaaagctc 32460
cctccgaaaa aatgggaaca tggtatcaaa ggcagtgccc gcaacacaca tcttgaacag 32520
atccatcagg atagtagctc gacacagccc ctgcagactt tggtcagctt gcttgctgca 32580
gcagtacact ctccacgtag catctccgct gatgaagtat tcgctatcgc agcgaccaaa 32640
aatacagcaa tcacaaggca gacgcaacag tctttcatcc agactgttca tgagaggctt 32700
tagaggtatg ggaaaaaatc caaagtgctc aaaataagca gcgctgggct cattctgaca 32760
ttcccccaac atgctgagtc gaaccatagc acagtcatac aaactcagct gtcggaattg 32820
atcttccatg attgagtttc tactgagata ttatctcaaa cttaaaactg ttgctcacca 32880
actctatgcg aacttgctca agaagctctt ggtttagggc gacctcttct ggtcgtcgga 32940
agttactgat ggaacaacaa gcgccgccca acttcaaatt tccagccgac ccaatccagt 33000
ggtctctcaa ctcacgcgca caagctacta tgcagtcctc actttcgtca aagtcagcag 33060
cgcctataga aatcaacaca ctgagtccac catcttcagc ttttaaggga taacagctga 33120
tagcaaactg gttctgagac cacggcaaag cacgtaggaa ttgctgttaa gttaatttcc 33180
aaacaccgct gaagcagctc tatggttgct ggacatatgt cctctgcata gaagctttga 33240
acataactta agacagggcc gggcacatga aacacaaaca gagaactata cacaatctgg 33300
gccatgatca ctcacattta aatagcagct gaaaagtggc tttcttcact tgggagcaaa 33360
attagcgaag actgtgccag aatgctcacg tcgaaaggcg gtgggtctcg cagaggcagg 33420
ttcggagctc taattaaaca caggtgggta atccagtcaa cgatgaggac cagctgaaaa 33480
gtggctttct tcacttggga gcaaaattag cgaagactgt gccagaatgc tcacgtcgaa 33540
aggcggtggg tctcgcagag gcaggttcgg agctctaatt aaacacaggt gggtaatcca 33600
gtcaacgatg aggactttta aaaaactgtc taaaactgaa gcagttaagt tagaggcaga 33660
cacagaaaaa actacagtta aactatcagt tgctgaaatt gaaaagcacc caataattat 33720
gcgcgagggc acaggcaata aaagtgttag cccctcggct aacgcgtcag ctaaaaaatc 33780
tttagctaaa gtatctactg gccgcgtggt aaaagtttga atataattta cgacaggagc 33840
tggcaagtga aactccacaa aaaaagtaaa tggctgcaca cacgccatta ttttgaaaat 33900
aagaagtact cacaaaatca gctggagctg ccgcaagtga aaaagaccag ctgaagtctt 33960
attttaaact gtaaaatata aaaaaaaaaa tagggcgtga acaaaaatga gaaaataata 34020
ccggatatga ctattaaggg cgtacactga aactgggtaa tatttgagaa aaagattaag 34080
ataatagctg aacaaatgtt gtgtgcagaa cacggaacaa tggtggcgaa aaaaaaaaac 34140
agtgtaagca catggcgcgc acgtacttcc gtgagaaaaa ttaaaaaaat ttacccagta 34200
taaggtgcgt cattagaccc gccttgtggc gcggttgtag ccctgccctt tgccccgccc 34260
cgcgcgccgc cccgcgcgcc gcccccgccg ccctcagccc cgcccagcgc cgccgcctcc 34320
gcgacgcgct ccgccccaca gttacgtcag cacgccacgc tcgccgtcgt tgcgtcataa 34380
atgacgtggc aaaaatgatt ggcagttgga ccgctgccat cagtgtactg tagattattg 34440
atgatg 34446
Bovine Adenovirus pV amino acid sequence
(SEQ ID NO: 2)
MASSRLIKEEMLDIVAPEIYKRKRPRRERAAPYAVKQEEKPLVKAERKIKRGSRKRALSGVDVPLPDDGFEDDEPHI
EFVSAPRRPYQWKGRRVRRVLRPGVAVSFTPGARSLRPSSKRVYDEVYADDDFLEAAAAREGEFAYGKRGREAAQAQ
LLPAVAVPEPTYVVLDESNPTPSYKPVTEQKVILSRKRGVGKVEPTIQVLASKKRRMAENEDDRGAGSVAEVQMREV
KPVTAALGIQTVDVSVPDHSTPMEVVQSLSRAAQVAQRLTQQQVRPSAKIKVEAMDLSAPVDAKPLDLKPVDVKPTP
TFVLPSFRSLSTQTDSLPAAVVVPRKPRVHRATRRTARGLLPYYRLHPSITPTPGYRGSVYTSSGVRLPAVRRRRRR
RTRRATPRLSAAAAAALLPGVRYHPSIRQAATVTRLRR
V.m1.2m3d3 amino acid sequence
(SEQ ID NO: 15)
MASSRLIKEEMLDIVAPEIYAGAAAPAAAAAPYAVKQEEKPLVKAERKIKRGSRKRALSGVDVPLPDDGFEDDEPHI
EFVSAPRRPYQWKGRRVRRVLRPGVAVSFTPGARSLRPSSKRVYDEVYADDDFLEAAAAREGEFAYGKRGREAAQAQ
LLPAVAVPEPTYVVLDESNPTPSYKPVTEQKVILSRKRGVGKVEPTIQVLASKKRRMAENEDDRGAGSVAEVQMREV
KPVTAALGIQTVDVSVPDHSTPMEVVQSISRAAQVAQRLTQQQVRPSAKIKVEAMDLSAPVDAKPLDLKPVDVKPTP
TFVLPSFRSLSTQTDSLPAAVVVPRKPRVHRATRRTARGLLPYYRLHPSITPTPGYRGSVYTSSGVRLPAVATPRLS
AAAAAALLPGVRYHPSIRQAATVTRLRR

1. A defective bovine adenovirus (BAV) vector comprising inverted terminal repeat sequences and BAV packaging sequences, wherein the BAV vector lacks pV functions.

2. The defective BAV vector of claim 1, wherein the BAV vector comprises one or more modifications of the nucleic acid encoding pV wherein the pV lacks nuclear localization functions and/or nucleolar localization functions.

3. The defective BAV vector of claim 1, wherein the BAV vector comprises a deletion of part or all of the coding region for pV.

4. (canceled)

5. The defective BAV vector of claim 1, wherein the BAV vector comprises a deletion corresponding to nucleotides 15068 to 16299 of SEQ ID NO:1.

6. The defective BAV vector of claim 1, wherein the BAV vector comprises
a) a deletion of nucleotides encoding amino acid residues 1-423 of the pV set forth in SEQ ID NO:2;
b) comprises a deletion of nucleotides encoding amino acid residues 21-50 and 380-389 of the pV set forth in SEQ ID NO:2;
c) a deletion of nucleotides encoding amino acid residues 21-50, 190-210 and 380-389 of the DV set forth in SEQ ID NO:2;
d) a deletion of nucleotides encoding amino acid residues 21-50 and 380-423 of the pV set forth in SEQ ID NO:2,
e) a deletion of nucleotides encoding amino acid residues 21-50, 190-210 and 380-423 of the pV set forth in SEQ ID NO:2,
f) a deletion of nucleotides encoding amino acid residues 21-50, 190-210 and 323-423 of the pV set forth in SEQ ID NO:2′
g) a deletion of nucleotides encoding amino acid residues 21-50 and 190-423 of the pV set forth in SEQ ID NO:2,
h) a deletion of nucleotides encoding amino acid residues 21-50, 101-210 and 380-423 of the DV set forth in SEQ ID NO:2,
i) a deletion of nucleotides encoding amino acid residues 3-100, 190-210 and 380-423 of the pV set forth in SEQ ID NO:2,
j) a deletion of nucleotides encoding amino acid residues 21-50, 81-120, 190-210 and 380-423 of the pV set forth in SEQ ID NO:2,
k) a deletion of nucleotides encoding amino acid residues 81-120, 190-210 and 380-423 of the pV set forth in SEQ ID NO:2, or
l) a deletion of nucleotides encoding amino acid residues 81-120, 190-210 and 390-423 of the pV set forth in SEQ ID NO:2.

7-17. (canceled)

18. The defective BAV vector of claim 1, wherein the BAV vector comprises one or more substitutions of the nucleic acid encoding pV such that the BAV pV lacks nuclear localization functions and/or nucleolar localization functions.

19. The defective BAV vector of claim 18, wherein substitution of the nucleic acid encoding pV results in the substitution of one or more of amino acid residues 21-50 or 380-389 of the pV set forth in SEQ ID NO:2.

20. (canceled)

21. The defective BAV vector of claim 1, wherein the BAV vector further comprises a deletion of all or part of the E3 region.

22. The defective BAV vector of claim 1, wherein the BAV vector further comprises nucleic acid encoding a heterologous transgene.

23. The defective BAV vector of claim 22, wherein the nucleic acid encoding the heterologous transgene is located in the E3 region.

24. The defective BAV vector of claim 22, wherein the heterologous transgene encodes a therapeutic polypeptide or a therapeutic nucleic acid.

25. The defective BAV vector of claim 22, wherein the heterologous transgene encodes a coagulation factor, a hormone, a cytokine, a lymphokine, an oncogene product, a tumor suppressor, a cell receptor, a ligand for a cell receptor, a protease inhibitor, an antibody, a toxin, an immunogenic polypeptide, an antibody, a dystrophin, a cystic fibrosis transmembrane conductance regulator (CFTR), siRNA, mRNA, miRNA, lncRNA, tRNA, or shRNA.

26. The defective BAV vector of claim 1, wherein the BAV vector is a BAV-3 vector.

27. A recombinant bovine adenovirus (rBAV) particle, wherein the rBAV particle comprises a rBAV genome comprising inverted terminal repeat sequences and BAV packaging sequences, wherein the BAV genome lacks pV functions.

28. The rBAV particle of claim 27, wherein the rBAV genome comprises one or more modifications of the nucleic acid encoding pV wherein the pV lacks nuclear localization functions and/or nucleolar localization functions.

29. The rBAV particle of claim 27, wherein the rBAV genome comprises a deletion of part or all of the coding region for pV.

30-52. (canceled)

53. A vaccine comprising a recombinant bovine adenovirus (rBAV) particle, wherein the rBAV particle comprises a rBAV genome comprising inverted terminal repeat sequences, BAV packaging sequences, and nucleic acid encoding a heterologous antigen; wherein the BAV genome lacks pV functions.

54-76. (canceled)

77. A pharmaceutical composition comprising the defective BAV vector of claim 1.

78-80. (canceled)

81. A mammalian cell comprising nucleic acid encoding a BAV pV, said cell is capable of providing BAV pV function.

82-88. (canceled)

89. A method for producing a defective BAV vector comprising introducing a BAV genome to the cell of claim 81 and culturing the cells under conditions where the defective BAV vector is produced, wherein the defective BAV vector lacks pV function.

90-115. (canceled)

116. A defective BAV vector prepared by the method of claim 89.

117. A pharmaceutical composition comprising the defective BAV vector of claim 116.

118. (canceled)

119. A method for treating a disease or disorder in an individual in need thereof comprising administering the pharmaceutical composition of claim 77 wherein the defective BAV vector of the rBAV particle comprises a heterologous transgene suitable for treating the disease or disorder.

120. A method for eliciting an immune response in an individual comprising administering the pharmaceutical composition of claim 77, wherein the defective BAV vector, the rBAV particle or the vaccine comprises a heterologous transgene encoding an antigen.

121-130. (canceled)

131. A kit comprising the defective BAV vector of claim 1.

132-138. (canceled)


SEQUENCE LISTING (37 sequences)

SEQ ID NO: 1 - DNA, 34446 bp, Bovine Adenovirus
catcatcaataatctacagtacactgatggcagcggtccaactgccaatcatttttgccacgtcatttatgacgcaacgacggcgagcgtggcgtgctgacgtaactgtggggcggagcgcgtcgcggaggcggcggcgctgggcggggctgagggcggcgggggcggcgcgcggggcggcgcgcggggcggggcgaggggcggagttccgcacccgctacgtcattttcagacattttttagcaaatttgcgccttttgcaagcatttttctcacatttcaggtatttagagggcggatttttggtgttcgtacttccgtgtcacatagttcactgtcaatcttcattacggcttagacaaattttcggcgtcttttccgggtttatgtccccggtcacctttatgactgtgtgaaacacacctgcccattgtttacccttggtcagttttttcgtctcctagggtgggaacatcaagaacaaatttgccgagtaattgtgcacctttttccgcgttaggactgcgtttcacacgtagacagactttttctcattttctcacactccgtcgtccgcttcagagctctgcgtcttcgctgccaccatgaagtacctggtcctcgttctcaacgacggcatgagtcgaattgaaaaagctctcctgtgcagcgatggtgaggtggatttagagtgtcatgaggtacttcccccttctcccgcgcctgtccccgcttctgtgtcacccgtgaggagtcctcctcctctgtctccggtgtttcctccgtctccgccagccccgcttgtgaatccagaggcgagttcgctgctgcagcagtatcggagagagctgttagagaggagcctgctccgaacggccgaaggtcagcagcgtgcagtgtgtccatgtgagcggttgcccgtggaagaggatgagtgtctgaatgccgtaaatttgctgtttcctgatccctggctaaatgcagctgaaaatgggggtgatatttttaagtctccggctatgtctccagaaccgtggatagatttgtctagctacgatagcgatgtagaagaggtgactagtcacttttttctggattgccctgaagaccccagtcgggagtgttcatcttgtgggtttcatcaggctcaaagcggaattccaggcattatgtgcagtttgtgctacatgcgccaaacctaccattgcatctatagtaagtacattctgtaaaagaacatcttggtgatttctaggtattgtttagggattaactgggtggagtgatcttaatccggcataaccaaatacatgttttcacaggtccagtttctgaagaggaaatgtgagtcatgttgactttggcgcgcaagaggaaatgtgagtcatgttgactttggcgcgccctacggtgactttaaagcaatttgaggatcacttttttgttagtcgctataaagtagtcacggagtcttcatggatcacttaagcgttcttttggatttgaagctgcttcgctctatcgtagcgggggcttcaaatcgcactggagtgtggaagaggcggctgtggctgggacgcctgactcaactggtccatgatacctgcgtagagaacgagagcatatttctcaattctctgccagggaatgaagcttttttaaggttgcttcggagcggctattttgaagtgtttgacgtgtttgtggtgcctgagctgcatctggacactccgggtcgagtggtcgccgctcttgctctgctggtgttcatcctcaacgatttagacgctaattctgcttcttcaggctttgattcaggttttctcgtggaccgtctctgcgtgccgctatggctgaaggccagggcgttcaagatcacccagagctccaggagcacttcgcagccttcctcgtcgcccgacaagacgacccagactaccagccagtagacggggacagcccaccccgggctagcctggaggaggctgaacagagcagcactcgtttcgagcacatcagttaccgagacgtggtggatgacttcaatagatgccatgatgttttttatgagaggtacagttttgaggacataaagagctacgaggctttgcctgaggacaatttggagcagctcatagctatgcatgctaaaatcaagctgctgcccggtcgggagtatgagttgactcaacctttgaacataacatcttgcgcctatgtgctcggaaatggggctactattagggtaacaggggaagcctccccggctattagagtgggggccatggccgtgggtccgtgtgtaacaggaatgactggggtgacttttgtgaattgtaggtttgagagagagtcaacaattagggggtccctgatacgagcttcaactcacgtgctgtttcatggctgttattttatgggaattatgggcacttgtattgaggtgggggcgggagcttacattcggggttgtgagtttgtgggctgttaccggggaatctgttctacttctaacagagatattaaggtgaggcagtgcaactttgacaaatgcttactgggtattacttgtaagggggactatcgtctttcgggaaatgtgtgttctgagactttctgctttgctcatttagagggagagggtttggttaaaaacaacacagtcaagtcccctagtcgctggaccagcgagtctggcttttccatgataacttgtgcagacggcagggttacgcctttgggttccctccacattgtgggcaaccgttgtaggcgttggccaaccatgcaggggaatgtgtttatcatgtctaaactgtatctgggcaacagaatagggactgtagccctgccccagtgtgctttctacaagtccagcatttgtttggaggagagggcgacaaacaagctggtcttggcttgtgcttttgagaataatgtactggtgtacaaagtgctgagacgggagagtccctcaaccgtgaaaatgtgtgtttgtgggacttctcattatgcaaagcctttgacactggcaattatttcttcagatattcgggctaatcgatacatgtacactgtggactcaacagagttcacttctgacgaggattaaaagtgggcggggccaagaggggtataaataggtggggaggttgaggggagccgtagtttctgtttttcccagactgggggggacaacatggccgaggaagggcgcatttatgtgccttatgtaactgcccgcctgcccaagtggtcgggttcggtgcaggataagacgggctcgaacatgttggggggtgtggtactccctcctaattcacaggcgcaccggacggagaccgtgggcactgaggccaccagagacaacctgcacgccgagggagcgcgtcgtcctgaggatcagacgccctacatgatcttggtggaggactctctgggaggtttgaagaggcgaatggacttgctggaagaatctaatcagcagctgctggcaactctcaaccgtctccgtacaggactcgctgcctatgtgcaggctaaccttgtgggcggccaagttaacccctttgtttaaataaaaatacactcatacagtttattatgctgtcaataaaattctttatttttcctgtgataataccgtgtccagcgtgctctgtcaataagggtcctatgcatcctgagaagggcctcatataccatggcatgaatattaagatacatgggcataaggccctcagaagggttgaggtagagccactgcagactttcgtggggaggtaaggtgttgtaaataatccagtcatactgactgtgctgggcgtggaaggaaaagatgtcttttagaagaagggtgattggcaaagggaggctcttagtgtaggtattgataaatctgttcagttgggagggatgcattcgggggctaataaggtggagtttagcctgaatcttaaggttggcaatgttgccccctaggtctttgcgaggattcatgttgtgcagtaccacaaaaacagagtagcctgtgcatttggggaatttatcatgaagcttggaggggaaggcatgaaaaaattttgagatggctttatggcgccccaggtcttccatgcattcgtccataataatagcaataggcccggttttggctgcctgggcaaacacgttctgagggtgggcgacatcatagttgtagtccatggtcaggtcttcataggacatgatcttaaaggcaggttttagggtgctgctttgaggaaccagagttcctgtggggccgggggtgtagttcccttcacagatttgggtctcccaagcaagcagttcttgcgggggtatcatgtcaacttgggggactataaaaaaaacagtttcgggaggtggttgaatgaggcccgtagacataaggtttctgaggagctgggattttccacaaccggttggtccgtagaccaccccaataacgggttgcatggtaaagtttaaagatttgcatgaaccgtcagggcgcagatatggcatggtggcattcatggcatctcttatcgcctgattatagtctgagagggcattgagtagggtggcgccccccatagccagtagctcgtccaaggaagaaaagtgtctaagaggtttgaggccttcagccatgggcatggactctaagcactgttgcatgagagcacatttgtcccaaagctcagagacgtggtctagtacatctccatccagcatagctctttgtttcttgggttggggtggctgttgctgtagggggcgagacggtgacggtcgatggcccgcagggtgcggtctttccagggcctgagcgtcctcgccagggtcgtctcggtgaccgtgaagggctgctgatgcgtctgtctgctgaccagcgagcgcctcaggctgagcctgctggtgccgaacttttcgtcgcctagctgttcagtggaataataacaagtcaccagaaggtcgtaggagagttgtgaggtggcatggcctttgctcgaagtttgccagaactctcggcggcggcagcttgggcagtagatgtttttaagggcatatagtttgggggctaagaagacagattcctggctgtgggcgtctccgtggcagcgggggcactgggtctcgcattccacaagccaagtcagctgagggttggtgggatcaaagaccagaggacggttattacctttcaggcggtgcttgcctcgggtgtccatgagttcctttcccctttgggtgagaaacatgctgtccgtgtctccgtagacaaatttgagaatccggtcttctaggggagtgcctctgtcttctaaatagaggatgtctgcccattcagagacaaaggctctagtccacgcgaggacaaatgaagctatgtgtgaggggtatctgttattaaatatgagagaggattttttttgcaaagtatgcaggcacagggctgagtcatcagcttccagaaaggtgattggtttgtaagtgtatgtcacgtgatggttctgggggtctcccagggtataaaagggggcgtcttcgtctgaggagctattgctagtgggtgtgcactgacggtgcttccgcgtggcatccgtttgctgcttgacgggtgagtaggtgatttttagctctgccatgacagaggagctcaggttgtcagtttccacgaaggcggtgcttttgatgtcgtaggtgccgtctgaaatgcctctaacatatttgtcttccatttggtcagaaaagacagtgactctgttgtctagcttagtggcaaagctgccatacagggcattggacagcagtttggcaatgcttctgagagtttggtttttctctttatccgccctttccttgggcgcaatgttaagttgcacgtagtctctagccagacactcccactggggaaatactgtggtgcgggggtcgttgagaatttggactctccagccgcggttatgaagcgtgatggcatccaaacaagttaccacttccccccgtagtgtctcgttggtccagcagaggcgacctccttttctggagcagaagggcggtataacgtccaagaatgcttctgggggtgggtctgcatcaatggtgaatatcgcgggcagtagggtgcgatcaaaatagtcaatgggtctgtgcaactgggttaggcggtcttgccagtttttaattgcaagcgctcgatcaaaggggttcaaaggttttcccgctgggaaaggatgggtgagggcgctggcatacatgccgcagatgtcatacacatagatggcttctgttaggacgcctatgtaggtaggatagcatcggccgccccgaatactttctctaacgtaatcatacatttcattggaaggggctagtagaaagttgcccagagagctcctgttgggacgctgggatcggtagactacctgtctgaagatggcatgggaattggagctgatggtgggcctttggaggacattgaaattgcagtggggcagccccactgacgtgtgaacaaagtccaaataagatgcttggagttttttaaccaattcggccgtaaccagcacgtccatagcacagtagtccaaggtgcgttgcacaatatcataggcacctgaattctcttgcagccagagactcttattgagaaggtactcctcgtcgctggaccagtagtccctctgaggaaaagaatctgcgtcggttcggtaggtacctaacatgtaaaattcatttacagctttgtaagggcagcagcctttttccacgggtaaagcgtaagcggcagctgcgttcctgagactcgtgtgcgtgagagcaaaggtatctcggaccatgaacttcacaaactgaaatttatagtctgctgaggtgggagtgccttcctcccagtctttgaagtcttttcgagcagcatgtgtggggttaggcagagcaaaagttaagtcattgaaaagaatcttgccacaacgaggcatgaaatttctactgactttaaaagcagctggaataccttgtttgttgttaatgacttgtgcggctagaacaatctcatcaaagccgtttatgttgtgccctacgacatagacttccaagaaagtcggttgccctttgagttcaagcgtacacagttcctcgaaaggaatgtcgctggcatggacatagcccagtttgaggcagaggttttctaagcacggattatctgccaggaactggcgccaaagcaaagtgctggcagcttcttgaagggcatcccgatactgtttaaacaagctgcctactttgtttctttgcgggttgaggtagtagaaggtatttgcttgctttggccagcttgaccacttttgctttttagctatgttaacagcctgttcgcatagctgcgcgtcaccaaacaaagtaaacacgagcataaaaggcatgagttgcttgccaaagctaccgtgccaagtgtatgtttccacatcatagacgacaaagaggcgccgggtgtcggggtgagcggcccaggggaaaaactttatttcttcccaccagtccgaagattgggtgtttatgtggtgaaagtaaaagtcccggcggcgagtgctgcaggtgtgcgtctgcttaaaatacgaaccgcagtcggcacatcgctggacctctgcgatggtgtctatgagatagagctttctcttgtgaataagaaagttgagggggaagggaaggcgcggcctgtcagcgcgggccgggatgcttgtaattttcagcttccccttgtatgttttgtaaacgcacatatttgcgttgcagaaccggacgagcgtgtcttggaatgaaaggatattttctggttttaaatcaaatgggcagtgctccaagtgcagttcaaaaaggtttcggagactgctggaaacgtctgcgtgatacttgacttccagggtggtcccgtcttcagtctgaccgtgcagccgtagggtactgcgtttggcgaccaggggcccccttggggctttctttaaaggggacgtcgagggccgaggggcggcctttgcctttcgggcctgaggggcggtagctggaccggatcgttgagttcgggcatgggttgcagctgttggcgcaggtctgatgcgtgctgcacgactctgcggttgattctctgaatctccgggtgttgggtgaatgctactggccccgtcactttgaacctgaaagagaggtcgacagagttaatagatgcatcgttaagctccgcctgtctaataatttcttccacgtcaccgctgtggtctcggtaagcaatgtctgtcataaaccgttcgatctcttcctcgtccagttctccgcgaccagctcggtggaccgtggctgccaagtccgtgctaatgcgtcgcatgagctgggaaaaggcattggttcccggttcattccacactctgctgtatataacagcgccatcttcgtctcgggctcgcatgaccacctggcccaagtttagctccacgtggcgagcaaagacggggctgaggcggaggtggtggtgcagataattgagagtggtggctatgtgctccacgatgaagaagtagatgacccatctgcggatggtcgactcgttaatgttgccctctcgctccagcatgtttatggcttcgtaaaagtccacagcgaagttaaaaaactgctcgttgcgggcggagactgtcagctcttcttgcaggagacgaatgacttcggctacggcggcgcggacttcttcggcaaaggagcgcggcggcacgtcctcctcctcctcttcttccccctccagcgggggcatctccagctctaccggttccgggctgggggacagggaaggcggtgcgggccgaacgacccgtcggcgtcgggtgggcaaggggagactctctatgaatcgctgcaccatctcgccccggcgtatccgcatctcctgggtaacggcacgcccgtgttctcggggtcggagctcaaaagctccgccccgcagttcggtcagaggccgcgccgcgggctggggcaggctgagtgcgtcaataacatgcgccaccactctctccgtagaggcggctgtttcgaaccgaagagactgagcatccacgggatcgctgaagcgttgcacaaaagcttctaaccagtcgcagtcacaaggtaggctgagcataggtgaggctcgctcggtgttgtttctgtttggcggcgggtggctgaggagaaaattaaagtacgcgcaccgcaggcgccggatggttgtcagtatgatgagatccctgcgacccgcttgttggattctgatgcggtttgcaaagccccaggcttggtcttggcatcgcccaggttcatgcactgttcttggaggaatctctctacgggcacgttgcggcgctgcgggggcagggtcagccatttcggtgcgtccaaacccacgcaatggttggatgagagccaagtccgctactacgcgctctgctaggacggcttgctggatctgccgcagcgtttcatcaaagttttccaagtcaatgaagcggtcgtaggggcccgcgtttatggtgtaggagcagtttgccatggtggaccagtccacaatctgctgatctacccgcaccgtttctcggtacaccagtcggctataggctcgcgtctcgaaaacatagtcgttgcaaacgcgcaccacgtattggtagccgattaggaagtgcggcggcgggtataagtagagcggccagttttgcgtggccggctgtctggcgcccagattccgtagcatgagtgtggggtatcggtacacgtgacgcgacatccaggagatgcccgcggccgaaatggcggccctggcgtactcccgggcccggttccatatattcctgagaggacgaaagattccatggtgtgcagggtctgccccgtaagacgcgcgcaatctctcgcgctctgcaaaaaacatacagatgaaacatttttggggcttttcagatgatgcatcccgctttacggcaaatgaagcccagatccgcggcagtggcgggggttcctgctgcggccgccggcgcgagcgttgactcaggcggtactaccgcgccccctggtgtcgagtgcggcgagggggaagggttagctcggctgtacgcgcacccggacacacacccgcgcgtgtgcgtgaagcgcgatgcggcggaggcgtacgttccccgggagaacttattccgcgaccgcagcggggaggaacccgaagggagccgagacctaaagtacaaggccggtcggcagttgcgcgccggcatgccccgaaagcgggtgctgaccgaaggggactttgaggtggatgagcgcactggcatcagctcagccaaagcccacatggaggcggccgatctagtgcgggcttacgagcaaacggtgaagcaagaggctaattttcaaaagtcatttaataaccacgtgcggacactgatctcccgcgaggagaccaccctgggtttgatgcacttgtgggactttgcggaggcatacgcgcagaaccccggcagcaagacccttacggcccaagtctttctcatcgtgcagcacttgcaagatgagggcatttttggggaagctttcttaagcatagcagagcccgagggacgatggatgctagatctgctaaacatattgcagtccattgtggtgcaagagcgccagctttcgctatctgaaaaggtagccgcggtgaactactccgtagttaccctgggcaaacattatgcccgcaagatctttaagagcccctttgtgccgcttgacaaggaggtgaagatcagtacattttatatgcgcgcggtgcttaaggtcctgggtctaagtcacgacctgggcatgtacagaaacgaaaaggtggagaagctagctagcataggcaggcgttcgggagatgagcgacgcggagctgctgttcaacctccgccgcgcactaaccactggcgattctgaagcattcgatgaaggcggggactttacctgggctccgccaactcgcgcgaccgcggcggccgctttgccggggcccgagtttgagagtgaagagacggacgatgaagtcgacgaatgagtgatgcggacccccgtatctttcagctggtcagtcggcaagagaccgtagccatggccgaagcgccccgaagcctgggccccgccccttccaatcctagtttgcaggctttattccaaagccagcccagcgccgagcaggagtggcacggcgtgctggagagagtcatggcccttaacaaaaatggagactttggctcgcagccccaggcgaaccggtttggagccatcctcgaagccgtggtgcccccgcgctccgatcccacccatgaaaaagtgctagctattgtgaatgcgctcttggagactcaggccatccgtcgcgatgaggccggacagatgtacaccgcgctgttgcagcgggtggccagatacaacagtgtgaatgtgcagggcaatttggacaggctgattcaggacgtgaaggaggctctggcgcagcgcgagcgcaccgggccgggggccggcctagggtctgtggtagccttgaatgccttcctgagcacacagccagcggtggtggagaggggccaggagaactatgtggcctttgtgagcgccttaaaactcatggtgaccgaggcgccgcagtctgaggtttaccaggccggacctagtttcttttttcaaaccagccggcacggttcgcagacggtaaacctcagtcaggcctttgataacttgcgacccctctggggcgtgcgcgcgccagtacacgagcgtactaccatctcctctctgctcacaccaaacacccgcttgctcttgctcctcattgcgccctttacggacagcgtgggcatatcccgggacagttacctggggcatctgctgaccctttaccgggagaccataggtaacactcgagttgatgagaccacgtacaacgagatcacggaagtgagtcgggccctgggcgccgaagacgcgtctaacttgcaagccactctcaactacttactcacaaataagcagagcaagttgccacaggagttttctctgagtcccgaagaggagcgggtgctgcgctacgtgcagcaatctgtcagtttatttttaatgcaggatggacacacggccaccactgctctagatcaggctgcggccaacatagcgccctcgttttacgcgtcccaccgcgactttataaaccgactgatggactatttccagcgagctgcggctatggcccctgactactttttacaggctgttatgaatccccactggctcccgccgccgggtttctttactcaggagtttgactttccggagcccaacgaaggcttcctgtgggatgatttggacagcgcgctcctacgcgcgcacgtaaaagaagaggaggatcaaggagctgtgggcggcacgccggcggcttcggcgcccgcgtctcgcgcgcacacaccaccgccgccgcccggtgccgcggacctctttgctcctaacgccttccgcaatgtgcaaaataacggcgtggatgaacttattgacggcttaagcagatggaagacttacgcccaggagaggcaggaagtcgttgagcggcacaggcgcagagaggcgcgtcgccgggcgcgcgaggcgcgtctagagtcgagcgatgatgacgacagcgacctagggccgtttctacggggcacggggcacctcgttcacaaccagtttatgcatctgaagccccggggtccccgccagttttggtaaccgcactgtattaagctgtaagtcctctcatttgacacttaccaaagccatggtcttgcttcgcctctgacactttctctccccccacacgcggcaccctacagcctaggggcgatgctccagcccgaactgcagccaattccgctgtcccgccgccggcttatgaggcggtggtggctggggccttccagacgctttctcttcgacgagatccacgtcccgccgcgatatgctgccgcgtctgcggggagaaacagtatccgttattccatgctgcccccgttgtatgacaccacgaagatataccttatcgacaacaaatcttcagacatccaaactctgaattaccaaaacgaccactcagattacctcactaccatcgtgcagaacagcgacttcacgcccctggaggctagcaaccacagcatcgagctagacgagcggtcccgctggggcggaaaccttaaaaccatcctttatacaaacctgcctaatatcacccagcacatgttttctaactcttttcgggtaaagatgatggcctcaaaaaaagacggcgtgccccagtacgagtggttccccctaaggctgcccgagggtaacttttctgagactatggtcattgacctcatgaacaatgccatcgtagagctgtacttggctttggggcgccaggagggcgtgaaggaagaggacatcggggtaaagatcgatacgcgcaactttagtctgggctatgacccgcagacccagttagtgacgcccggcgtatacaccaatgaagctatgcatgcggacatcgtgttgctgccgggctgtgctatagactttacgcactcccgattaaacaacctcttgggcatacgcaagcgttttccgtaccaagagggcttcgtcatctcctatgaggaccttaaggggggtaacatccccgctttgatggacgtggaggagtttaacaagagcaagacggttcgagctttgcgggaggaccccaaggggcgcagttatcacgtgggcgaagacccagaagccagagaaaacgaaaccgcctaccgcagctggtacctggcttacaattacggggacccagaaaaaggggtgcgggccaccacactgctgactaccggcgacgtgacctgcggggtggaacagatctactggagcttgccggacatggcactggacccagtcactttcaaggcttcgctgaaaactagcaattaccccgtggtgggcacagaacttttgccactggtgccgcgtagcttttataacgctcaggctgtgtactcacagtggatacaagaaaaaactaaccagacccacgttttcaatcgctttcccgaaaatcagatcttggtgcggccccctgcgcctaccatcacgtccataagtgaaaataagcccagcttgacagatcacggaatcgtgccgctccggaaccgcttggggggcgtgcaacgtgtgactttgactgacgcgcggcgaagatcctgcccctacgtctacaagagcttaggcattgtgacgccgcaagtgctatctagccgcacgttttaagcagacaggggcacagcagccgttttttttttttttttttcgctccaccagggactgtcaggaacatggccattctaatctctcctagcaataacacgggctggggcctgggatgcaataagatgtacgggggcgctcgcatacgttcagacttgcatccagtgaaggtgcggtcgcattatcgggccgcctggggcagccgcaccggtcgggtgggtcgccgcgcaaccgcagctttagccgatgccgtcgcggccaccggtgatccggtggccgacacaatcgaggcggtggtggctgacgcccgccagtaccggcgccgcagacggcgaggggtgcgccgagtcagaaggttgcgtcggagcccccgcactgccctgcagcgacgggttcgtagcgtacgccgacaagtggcgagggcccgcagggtgggccggcgcgcggccgctatcgcagcagacgcggccatggccatggcggcgccagctcggcgacgccgtaacatctactgggtacgcgatgcggcaaccggagcccgcgttccggtgacaacccggcctacggtcagcaacaccgtttgaaatgtctgctacttttttttgcttcaataaaagcccgccgactgatcagccacaccttgtcacgcagaattctttcaaaccattgcgctctcagcgcgcgcgccgataaacccactgtgatggcctcctctcggttgattaaagaagaaatgttagacatcgtggcgcctgagatttacaagcgcaaacggcccaggcgagaacgcgcagcaccgtatgctgtgaagcaggaggagaagcctttagtaaaggcggagcgcaaaattaagcgcggctccagaaagcgggccttgtcaggcgttgacgttcctctgcccgatgacggctttgaggacgacgagccccacatagaatttgtgtctgcgccgcgtcggccctaccagtggaagggcaggcgggtgcgccgggttttgcgtcccggcgtggccgttagtttcacgcccggcgcgcgctccctccgtccgagttccaagcgggtgtatgacgaggtgtacgcagacgacgacttcttagaagcggccgcggcccgtgagggggagtttgcttacggaaagcggggacgcgaggcggcccaggcccagctgctaccggctgtggccgtgccggaaccgacttacgtagttttggatgagagcaaccccaccccgagctacaagcctgtaaccgagcagaaagttattctttcccgcaagcggggtgtggggaaggtagagcctaccatccaggttttagctagcaagaagcggcgcatggccgagaatgaggatgaccgcggggccggctccgtggccgaagtgcagatgcgagaagttaaaccggtaaccgctgccttgggtattcagaccgtggatgttagcgtgcccgaccacagcactcccatggaggtcgtgcagagtctcagtcgggcggctcaagtagctcaacgcctgacccaacaacaggtgcggccttcggctaagattaaagtggaggccatggatctttctgctcccgtagacgcaaagcctcttgacttaaaacccgtggacgtaaagccgaccccgaccttcgtgcttcccagctttcgttcactcagcacccaaactgactctttgcccgcggcagtggtcgtgccgcgcaagccccgcgtgcaccgtgctactaggcgtactgcgcgcggcttgctgccctattaccgcctgcatcctagcatcacgccgacaccgggttaccgaggatctgtctacacgagctcgggtgtgcgcctgcccgccgtccgggcgccgccgtcgccgccgtacccgcagggcgactccccgcctcagcgctgccgcggccgcggcgctgctgcccggcgtgcgctatcaccctagcatccgccaagcggccacagtaacccggctccgccgttaagcgctgtgaaactgcaacaacaacaacaaaaataaaaaaaagtctccgctccactgtgcaccgttgtccatcggctaataaagtcccgctttgtgcgccgcaggaaccactatccgtaacctgcgaaaatgagtccccgcggaaatctgacttacagactgagaataccggtcgccctcagtggccggcgccggcgccgaacaggcttgcgaggagggtctgcgtacctgctcggccgccgcagaaggcgcgcgggcggcggccgcctgcgcgggggcttccttcccctcctggctcccatcattgcagccgccatcggcgcaatccccggcatcgcatcagtggccattcaggcggcccacaacaaatagggacagtgtaaagaaagctcaatctcaataaaacaaaccgctcgatgtgcataacgctctcggcctgcaacttctgctgcttacgtctttgaccaaagtcactactgttttccttttacccagagccggcgccagccccacacagcttgttaacacgccatggacgaatacaattacgcggctcttgctccccggcaaggctcccgacccatgctgagccagtggtccggcatcggcacgcacgaaatgcacggcggacgttttaatctgggcagtttgtggagcgggatcaggaatgtgggcagcgcgttaagaactggggctctcgggcctggcacagcaatgcgggcaagcgttgcgcgcccagctgaaaaagacgggcttgcaagaaaagatattgagggcgttagcgccggtatccacggagccgtggatctgggccgtcagcagctagagaaagctattgagcagcgcctagagcgtcgccccaccgctgccggtgtggaagaccttccgcttcccccgggaacagtcttagaagctgatcgtttaccgccctcctacgccgaagcggtggctgagcgcccgccgccggctgacgttctcctgcccgcatcctcaaagccgccggtggcggtggtgaccttgcccccgaaaaagagagtgtctgaagagcctgtggaggaagttgtgattcgttcctccgcaccgccgtcgtacgacgaggttatggcaccgcagccgactctggtagccgagcagggcgccatgaaagcagtgcccgtgattaagccggctcaaccttttaccccagctgtgcacgaaacgcaacgcatagtgaccaacttgccaatcaccacagctgtgacacggcgacgcgggtggcagggcactctgaatgacatcgtgggcctcggcgttcgtaccgtgaagcgccggcggtgctattgagggggcgcgcagcggtaataaagagaacataaaaaagcaggattgtgttttttgtttagcggccactgactctccctctgtgtgacacgtcctccgccagagcgtgattgattgaccgagatggctaccccgtcgatgctgccgcaatggtcctactgcacatcgccggtcaggacgcgtccgagtacctgtcccccggcttggtgcaattcgcacaagccaccgaatcctactttaacattgggaacaagtttagaaaccccaccgtcgccccgacgcacgatgtcaccacggagcgttcgcagcgtctgcagctccgcttcgtgcccgtagaccgggaggacacacagtactcctacaaaacccgcttccagctagccgtgggcgacaaccgggtgctggacatggccagcacgtattttgacatccgcggtacgctggagaggggcgccagtttcaagccttacagcggcacggcctacaactcctttgcccccaacagtgcccctaacaatacgcagtttaggcaggccaacaacggtcatcctgctcagaccatagctcaagcttcttacgtggctaccatcggcggtgccaacaatgacttgcaaatgggtgtggacgagcgtcagcagccggtgtatgcgaacactacgtaccagccggaacctcagctcggcattgaaggttggacagctggatccatggcggtcatcgatcaagcaggcgggcgggttctcaggaaccctactcaaactccctgctacgggtcctatgctaagccgactaacgagcacgggggcattactaaagcaaacactcaggtggagaaaaagtactacagaacaggggacaacggtaacccggaaacagtgttttatactgaagaggctgacgtgctaacgcccgacacccaccttgttcacgcggtaccggccgcggatcgggcaaaggtggaggggctatctcagcacgcagctcccaacaggccgaactttatcggctttcgggactgctttgtaggcttgatgtattataacagcgggggcaacctgggcgtcttagcgggtcaatcctctcagctgaatgccgtggtagacctgcaagaccgcaacactgagctttcctatcagatgcttcttgcaaacacgacggacagatcccgctattttagcatgtggaaccaagccatggactcgtacgacccggaggtcagggtgatagataacgtgggcgtagaggacgagatgcctaattactgctttccgttgtcgggggttcagattggaaaccgtagccacgaggttcaaagaaaccaacaacagtggcaaaatgtagctaatagtgacaacaattacataggcaaggggaacctaccggccatggagataaatctagcggccaatctctggcgttcctttttgtacagtaatgtggcgttgtacttgccagacaaccttaaattcacccctcacaacattcaactcccgcctaacacgaacacctacgagtacatgaacgggcgaatccccgttagcggccttattgatacgtacgtaaatataggcacgcggtggtcgcccgatgtgatggacaacgtgaatccctttaaccaccaccgcaactcgggcctgcgttaccgctcccagctgctgggcaacggccgcttctgcgactttcacattcaggtgccacaaaagttttttgctattcgaaacctgcttctcctgcccggcacgtacacttacgagtggtcctttagaaaggacgtaaacatgatccttcagagcactctgggcaatgatctgcgggtcgatggggccactgttaatattaccagcgtcaacctctacgccagcttctttcccatgtcacataacaccgcttccactttggaagctatgctccgcaacgacactaatgaccagtcttttaatgactatctctcggcggctaacatgttgtatcccattccgcccaatgccacccaactgcccatcccctcacgcaactgggcagcgttccgtggctggagtctcacccggctaaaacagagggagacaccggcgctggggtccccgttcgatccctatttcacctattcgggcaccatcccgtacctggacggcactttttacctcagccacacctttcgcaaggtggccatccagtttgactcttctgtgacctggcccggcaatgacaggcttttaacccctaacgagttcgaaataaaaataagtgtggacggtgaaggctacaacgtggctcagagcaatatgactaaggactggttcctggtgcagatgctagcgaattacaacataggctaccagggatatcacctgcccccggactacaaggacaggacattttccttcctgcataacttcatacccatgtgccgacaggttcccaacccagcaaccgagggctactttggactaggcatagtgaaccatagaacaactccggcttattggtttcgattctgccgcgctccgcgcgagggccacccctacccccaactggccttaccccctcattgggacccacgccatgccctccgtgacccagagagaaagtttctctgcgaccgcaccctctggcgaatccccttctcctcgaacttcatgtccatggggtccctcacagatctcggacagaacctactgtatgccaatgccgcgcatgccctagacatgacttttgagatggatcccatcaatgagcccactctgctgtacgttctgtttgaggtgtttgacgtggcccgcgttcaccagccccacagaggcgtgatcgaagtggtgtacttgagaacgccattctcagccggcaacgctaccacataagtgccggcttccctctcaggccccgcgatgggttctcgggaagaggagctgagattcatccttcacgatctcggtgtggggccatacttcctcggcactttcgataaacactttccggggttcatctccaaagaccgaatgagctgtgccatagtcaacactgccggacgcgaaaccgggggcgtgcattggctggccatggcttggcacccagcctcgcagaccttttacatgtttgaccctttcggtttctcggatcaaaagctaaagcaaatttacaactttgagtatcagggcctcctaaagcgcagcgccctgacttccactgctgaccgctgcctgacccttattcaaagcactcaatctgtccagggacccaacagcgccgcctgcggtctgttctgctgcatgttcctccacgcctttgtccgctggccgcttagggccatggacaacaatcccaccatgaacctcatccacggagttcccaacaacatgttggagagccccagctcccaaaatgtgtttttgagaaaccagcaaaatctgtaccgtttcctaagacgccactccccccattttgttaagcatgcggctcaaattgaggctgacaccgcctttgataaaatgttaacaaattagaccgtgagccatgattgcagaagcatgtcattttttttttattgtttaaaataaaaacaacacataacatctgccgcctgtcctcccgtgatttcttctgctttatttgcaaatggggggcaccttaaaacaaagagtcatctgcatcgtactgatcgatgggcagaataacattctgatgctggtactgcgggtcccagcggaattcgggaatggtaatgggggggctctgtttaaccagcgcggaccacatctgcttaaccagctgcaaggctgaaatcatatctggagccgaaatcttgaaatcgcagtttcgctgggcattagcccgcgtctgccggtacacagggttacagcactgaaatactaacaccgatgggtgttctacgctggccaggagtttgggatcttctacgaggctcttatctaccgcagagcccgcgttgatattaaagggcgttatcttgcatacctgacggcctaggaggggcaattgggagtgaccccagttacaatcacactttaaaggcataagcagatgagttccggcactttgcatcctggggtaacaggctttctgaaaggtcatgatctgccagaaagcctgcaaagccttgggcccctcgctgaaaaacataccacaagactttgaggtaaagctgccggccggcaaagcggcgtcaaagtgacagcaagccgcgtcttcattctttagctgcactacgttcatattccaccggttggtggtgatctttgtcttatgcggggtctcttttaaagcccgctgcccattttcgctgttcacatccatctctatcacttggtctttggtaagcataggcaggccatgcaggcagtgaagggccccgtctcccccctcggtacactggtggcgccagaccacacagcccgtggggctccacgaggtcgtccccaggcctgcgacttttaacacaaaatcatacaagaagcggcccataatagttagcacggttttctgagtactgaaagtaagaggcaggtacactttagactcattaagccaagcttgtgcaaccttcctaaaacactcgagcgtgccagtgtcgggcagcaaggttaagtttttaatatccactttcaaaggcacacacagccccactgctaattccatggcccgctgccaagcaacttcgtcggcttccagcaaggcccggctggccgccggcagggcgggagcggcggcctcagcggctggggctgaaggtttgaaaatcttggcgcgcttaacggctgtgacatcttcggcggggggctcagcgatcggcgcgcgccgtttgcggctgacttttttccggggcgtctcatctatcactaaggggttctcgtccccgctgctgtcagccgaactcgtggctcgcgttaagtcaccgctgcgattcattattctctcctagataacgacaacaaatggcagagaaaggcagtgaaaatcagcggccagagaacgacactgagctagcagcggtttcagaagccctaggcgcggccgcttcggccccctcacgtaactccccgactgacacggattcaggggtggaaatgacgcccaccagcagccccgagccgcccgccgctcccccaagttcgcctgccgcagcacctgcccctcagaagaaccaggaggagctctcttcccccgagcccgcggtagcagcagcggagccagaagccgcttcgcggcccagaccacccacacccaccgttcaggtcccgcgggagccgagcgaggatcaacctgacggacccgcgacgaggccttcgtacgtgagcgaggattgcctcatccgccatatctctcgccaggctaacattgttagagacagcctggcagaccgctgggagttagagcccaccgtgtcggctctctccgaggcttacgaaaagctcctcttttgtcccaaggtaccacccaagaagcaagagaatggcacttgcgaacctgaacctcgcgttaattttttccccacctttgtagtgcccgaaactttagccacgtaccacatctttttccaaaaccaaaaaatccccctgtcttgtcgcgccaaccgcacccacacagacaccatcatgcacctctactcgggggactccttaccgtgcttccccacgctgcagctggtcaacaaaatctttgaaggcttgggctcagaggagcggcgcgcagccaactcgctgaaagatcaagaggataacagcgcgttagttgagctcgaaggggacagtccccgactggctgtggttaagcgcacactgtctttgacacatttcgcctaccctgccataacactaccgcctaaggtgatggcagctgtcactggcagcctcattcatgaatcagcagcgaccgccgaaccggaagctgaggcgctgccagaagccgaggagcccgtggttagtgaccctgaacttgctcgctggttggggctcaacttacaacaggagcccgaggccacggcccaggctttggaagaaagacgcaagattatgttggcagtatgcttagtcacacttcagctcgagtgcctgcacaagtttttttcttcagaggatgtcatcaaaaagctgggagagagcctccactacgcctttcgccacggctacgtgcgccaagcctgctccatttctaacgtggaactaacgaacatcgtctcatacctgggtatcttgcacgaaaaccgcttgggacagagtaccctacacgccacccttaaagacgagaaccgcagagactacatcagagacacagtctttctctttctggtttatacttggcagactgccatgggcatttggcagcagtgcctcgagactgagaacgtaaaagaacttgaaaagctcttgcaaaaaagcaagagggctctctggacgggcttcgacgagctcaccatagctcaagacctagctgacatagtgttcccccccaaattcttgcacaccttgcaagccggcctgccagaccttacatcccagagtctccttcacaactttcgctccttcattttcgaacgctcgggcattctacccgccatgtgcaatgcactgcccaccgacttcatccctatcagctaccgggagtgccctccaactttctgggcctacacctacctctttaaactggccaattacctcatgtttcactccgacatcgcttacgatcggagcggccccggtctcatggaatgctactgtcgctgcaacctgtgcagtcctcaccgctgcttggcgaccaaccccgccctgctcagcgagacccaagttatcggtaccttcgagattcagggccctcctgctcaagacggacagccgaccaaaccgcccctcaggctgactgcaggtctctggacttccgcctacctgcgcaaatttgtaccgcaagacttcaacgcccacaaaatagccttctacgaagaccaatccaaaaagccgaaagtgacccccagcgcttgtgtcatcactgaagaaaaagttttagcccaattgcatgaaattaaaaaagcgcgggaagactttcctcttaaaaaggggcacggagtgtatctggaccctcagaccggcgaggagctgaacggacccgcaccctccgcagctaggaatgaaaccccgcagcatgtcggcagccgggccttccgcggctcaggcttcggagggccaacagctgccgccacagacagcggggctgcagccgagcaagagggctgtgaggaaggtagtagcttctctgaatcccaccgccgccctggaagacatatccgagggggaggaaggcttccccctgacggacgaggaagacggggacaccctggagagcgatttcagcgacttcacggacgaagacgtcgaggaggaggatatgatttcgataccccgcgaccaggggcactccggcgagctcgaggagggcgaaattcccgcaacggtagcggcgacggcggtcaagaagggccagggcaagaagagtaggtgggaccagcaggtccgctccacagcgcctctaaagggcgctagaggtaagaggagctacagctcctggaaacccctcaagcccactatcctttcatgcttactgcagagctccggcagcactgccttcactcgccgctatctgctttttcgccatggcgtgtccgttccctccagggtaattcattactataattcttactgcagacccgaagctgaccaaaaccgccactcagagcaaaaagagccgccggagtgccagcgcggcgcgccctcgccctcctcctcttcctcccaagcgtgctcgggcgccccgccgccccaaaggccagcgccatcaggccgacgacgcaagcaccgagggccgcgacaagcttcgggagctgatctttcccactctctatgccatattccaacaaagtcgcgctcagcggtgtcacctcaaagtgaaaaatagatccttacgttcactgacgcgcagctgcctctaccacaacaaggaggaacagctccagcgaaccctagcagactccgaggcgcttctcagtaaatactgctctgcagctccgacacgattctcgccgccctcttataccgagtctcccgccaaggacgaatccggacccgcctaaactctcagcatgagcaaagaaattcccacaccttatgtttggacctttcaacctcagatgggagcggccgcaggtgccagtcaagattactcgacccgcatgaattggttcagcgcgggacctgatatgatccacgacgttaacaacattcgtgacgcccaaaaccgcatccttatgactcagtcggccattaccgccactcccaggaatctgattgatcccagacagtgggccgcccacctcatcaaacaacccgtggtgggcaccacccacgtggaaatgcctcgcaacgaagtcctagaacaacatctgacctcacatggcgctcaaatcgcgggcggaggcgctgcgggcgattactttaaaagccccacttcagctcgaacccttatcccgctcaccgcctcctgcttaagaccagatggagtctttcaactaggaggaggctcgcgttcatctttcaaccccctgcaaacagattttgccttccacgccctgccctccagaccgcgccacgggggcataggatccaggcagtttgtagaggaatttgtgcccgccgtctacctcaacccctactcgggaccgccggactcttatccggaccagtttatacgccactacaacgtgtacagcaactctgtgagcggttatagctgagattgtaagactctcctatctgtctctgtgctgcttttccgcttcaagccccacaagcatgaaggggtttctgctcatcttcagcctgcttgtgcattgtcccctaattcatgttgggaccattagcttctatgctgcaaggcccgggtctgagcctaacgcgacttatgtttgtgactatggaagcgagtcagattacaaccccaccacggttctgtggttggctcgagagaccgatggctcctggatctctgttcttttccgtcacaacggctcctcaactgcagcccccggggtcgtcgcgcactttactgaccacaacagcagcattgtggtgccccagtattacctcctcaacaactcactctctaagctctgctgctcataccggcacaacgagcgttctcagtttacctgcaaacaagctgacgtccctacctgtcacgagcccggcaagccgctcaccctccgcgtctcccccgcgctgggaactgcccaccaagcagtcacttggttttttcaaaatgtacccatagctactgtttaccgaccttggggcaatgtaacttggttttgtcctcccttcatgtgtacctttaatgtcagcctgaactccctacttatttacaacttttctgacaaaaccggggggcaatacacagctctcatgcactccggacctgcttccctctttcagctctttaagccaacgacttgtgtcaccaaggtggaggacccgccgtatgccaacgacccggcctcgcctgtgtggcgcccactgctttttgccttcgtcctctgcaccggctgcgcggtgttgttaaccgccttcggtccatcgattctatccggtacccgaaagcttatctcagcccgcttttggagtcccgagccctataccaccctccactaacagtccccccatggagccagacggagttcatgccgagcagcagtttatcctcaatcagatttcctgcgccaacactgccctccagcgtcaaagggaggaactagcttcccttgtcatgttgcatgcctgtaagcgtggcctcttttgtccagtcaaaacttacaagctcagcctcaacgcctcggccagcgagcacagcctgcactttgaaaaaagtccctcccgattcaccctggtcaacactcacgccggagcttctgtgcgagtggccctacaccaccagggagcttccggcagcatccgctgttcctgttcccacgccgagtgcctccccgtcctcctcaagaccctctgtgcctttaactttttagattagctgaaagcaaatataaaatggtgtgcttaccgtaattctgttttgacttgtgtgcttgatttctccccctgcgccgtaatccagtgcccctcttcaaaactctcgtaccctatgcgattcgcataggcatattttctaaaagctctgaagtcaacatcactctcaaacacttctccgttgtaggttactttcatctacagataaagtcatccaccggttaacatcatgaagagaagtgtgccccaggactttaatcttgtgtatccgtacaaggctaagaggcccaacatcatgccgcccttttttgaccgcaatggctttgttgaaaaccaagaagccacgctagccatgcttgtggaaaagccgctcacgttcgacaaggaaggtgcgctgaccctgggcgtcggacgcggcatccgcattaaccccgcggggcttctggagacaaacgacctcgcgtccgctgtcttcccaccgctggcctccgatgaggccggcaacgtcacgctcaacatgtctgacgggctatatactaaggacaacaagctagctgtcaaagtaggtcccgggctgtccctcgactccaataatgctctccaggtccacacaggcgacgggctcacggtaaccgatgacaaggtgtctctaaatacccaagctcccctctcgaccaccagcgcgggcctctccctacttctgggtcccagcctccacttaggtgaggaggaacgactaacagtaaacaccggagcgggcctccaaattagcaataacgctctggccgtaaaagtaggttcaggtatcaccgtagatgctcaaaaccagctcgctgcatccctgggggacggtctagaaagcagagataataaaactgtcgttaaggctgggcccggacttacaataactaatcaagctcttactgttgctaccgggaacggccttcaggtcaacccggaagggcaactgcagctaaacattactgccggtcagggcctcaactttgcaaacaacagcctcgccgtggagctgggctcgggcctgcattttccccctggccaaaaccaagtaagcctttatcccggagatggaatagacatccgagataatagggtgactgtgcccgctgggccaggcctgagaatgctcaaccaccaacttgccgtagcttccggagacggtttagaagtccacagcgacaccctccggttaaagctctcccacggcctgacatttgaaaatggcgccgtacgagcaaaactaggaccaggacttggcacagacgactctggtcggtccgtggttcgcacaggtcgaggacttagagttgcaaacggccaagtccagatcttcagcggaagaggcaccgccatcggcactgatagcagcctcactctcaacatccgggcgcccctacaattttctggacccgccttgactgctagtttgcaaggcagtggtccgattacttacaacagcaacaatggcactttcggtctctctataggccccggaatgtgggtagaccaaaacagacttcaggtaaacccaggcgctggtttagtcttccaaggaaacaaccttgtcccaaaccttgcggatccgctggctatttccgacagcaaaattagtctcagtctcggtcccggcctgacccaagcttccaacgccctgactttaagtttaggaaacgggcttgaattctccaatcaagccgttgctataaaagcgggccggggcttacgctttgagtcttcctcacaagctttagagagcagcctcacagtcggaaatggcttaacgcttaccgatactgtgatccgccccaacctaggggacggcctagaggtcagagacaataaaatcattgttaagctgggcgcgaatcttcgttttgaaaacggagccgtaaccgccggcaccgttaacccttctgcgcccgaggcaccaccaactctcactgcagaaccacccctccgagcctccaactcccatcttcaactgtccctatcggagggcttggttgtgcataacaacgcccttgctctccaactgggagacggcatggaagtaaatcagcacggacttactttaagagtaggctcgggtttgcaaatgcgtgacggcattttaacagttacacccagcggcactcctattgagcccagactgactgccccactgactcagacagagaatggaatcgggctcgctctcggcgccggcttggaattagacgagagcgcgctccaagtaaaagttgggcccggcatgcgcctgaaccctgtagaaaagtatgtaaccctgctcctgggtcctggccttagttttgggcagccggccaacaggacaaattatgatgtgcgcgtttctgtggagccccccatggttttcggacagcgtggtcagctcacatttttagtgggtcacggactacacattcaaaattccaaacttcagctcaatttgggacaaggcctcagaactgaccccgtcaccaaccagctggaagtgcccctcggtcaaggtttggaaattgcagacgaatcccaggttagggttaaattgggcgatggcctgcagtttgattcacaagctcgcatcactaccgctcctaacatggtcactgaaactctgtggaccggaacaggcagtaatgctaatgttacatggcggggctacactgcccccggcagcaaactctttttgagtctcactcggttcagcactggtctagttttaggaaacatgactattgacagcaatgcatcctttgggcaatacattaacgcgggacacgaacagatcgaatgctttatattgttggacaatcagggtaacctaaaagaaggatctaacttgcaaggcacttgggaagtgaagaacaacccctctgcttccaaagctgcttttttgccttccaccgccctataccccatcctcaacgaaagccgagggagtcttcctggaaaaaatcttgtgggcatgcaagccatactgggaggcgggggcacttgcactgtgatagccaccctcaatggcagacgcagcaacaactatcccgcgggccagtccataattttcgtgtggcaagaattcaacaccatagcccgccaacctctgaaccactctacacttactttttcttactggacttaaataagttggaaataaagagttaaactgaatgtttaagtgcaacagacttttattggttttggctcacaacaaattacaacagcatagacaagtcataccggtcaaacaacacaggctctcgaaaacgggctaaccgctccaagaatctgtcacgcagacgagcaagtcctaaatgttttttcactctcttcggggccaagttcagcatgtatcggattttctgcttacacctttttagacagcagtttacactcatttccgttaaaggattacaactgcggcatatgagaattaagtatatacaactattgccctttacccacaaacactccccccacggggtgcacctgatgtagctgccctcctcaatcatgaaagtgctattaaagtaaattaaatgaacattattcacatacacgcttcccacataggccaaaaaaacagaggacaactttgacagctcccgcctgaaataccaatacactctatcaaactgcgcaccgtgcacgcactgctttaccaggccttgaaagtaaacagcggcggaccgacactgcaagcttctaggctttgggcagtggcagtgaatatatagccactcctccccatgcacgtagtaggaacgccgcttcccgggaatcacaaatgacaagcagtagtcacagaggcaactagtcaagtgagcgtcctcctgaggcatgattaccttccatggaatgggccagtgaatcatagtggcaaagccagctgcatctggagcgctgcgaaccttggctacatgtggtgattggcgacgcagatggagacaggaccttgcattctgaagaccactgcaacagcttctgcgtacgcttgtatttacagtacataaaaaagcacttttgccacagagcggtcttactcaaccgacagcttttttctttctgacgctgccttctgctactcaggtagtacaagtccaaaagagccaaacggacactcaaatccgggttatctcgatgctgaagccagagtccaaaagtaaccacgctaaaagcctgcatccatattttgtaactgctgtaactccatcccagagccgggcaccgcacttggtccaccatagctgcaaacaaacgggacaattaaggaaagtaaaatgagcgctgggggcggactcttctcccgttcgtaggaaacagccacgtatcaaacacccttttcaacactggctctccagccgctactcgttgaattaatttgtccctgtgctcaaacaacccacactggtaacggtggtcgctaggcaaacatgtcaaatagcacataatcatttccttcactttaagcaaacatcgactagcagacacttcacttaattcagcacagtcatagcaaggaatgattatacacttgtcatctaatccactgcccatgtacacattgccccaggcaaaagtgggcagggactttaagagctgattgctcgccccgacatagttggtaaaatacagcaaatgcaccttgttaacatacacactccccacatagtaaatataccgagtagacagcttagaaagctccctccgaaaaaatgggaacatggtatcaaaggcagtgcccgcaacacacatcttgaacagatccatcaggatagtagctcgacacagcccctgcagactttggtcagcttgcttgctgcagcagtacactctccacgtagcatctccgctgatgaagtattcgctatcgcagcgaccaaaaatacagcaatcacaaggcagacgcaacagtctttcatccagactgttcatgagaggctttagaggtatgggaaaaaatccaaagtgctcaaaataagcagcgctgggctcattctgacattcccccaacatgctgagtcgaaccatagcacagtcatacaaactcagctgtcggaattgatcttccatgattgagtttctactgagatattatctcaaacttaaaactgttgctcaccaactctatgcgaacttgctcaagaagctcttggtttagggcgacctcttctggtcgtcggaagttactgatggaacaacaagcgccgcccaacttcaaatttccagccgacccaatccagtggtctctcaactcacgcgcacaagctactatgcagtcctcactttcgtcaaagtcagcagcgcctatagaaatcaacacactgagtccaccatcttcagcttttaagggataacagctgatagcaaactggttctgagaccacggcaaagcacgtaggaattgctgttaagttaatttccaaacaccgctgaagcagctctatggttgctggacatatgtcctctgcatagaagctttgaacataacttaagacagggccgggcacatgaaacacaaacagagaactatacacaatctgggccatgatcactcacatttaaatagcagctgaaaagtggctttcttcacttgggagcaaaattagcgaagactgtgccagaatgctcacgtcgaaaggcggtgggtctcgcagaggcaggttcggagctctaattaaacacaggtgggtaatccagtcaacgatgaggaccagctgaaaagtggctttcttcacttgggagcaaaattagcgaagactgtgccagaatgctcacgtcgaaaggcggtgggtctcgcagaggcaggttcggagctctaattaaacacaggtgggtaatccagtcaacgatgaggacttttaaaaaactgtctaaaactgaagcagttaagttagaggcagacacagaaaaaactacagttaaactatcagttgctgaaattgaaaagcacccaataattatgcgcgagggcacaggcaataaaagtgttagcccctcggctaacgcgtcagctaaaaaatctttagctaaagtatctactggccgcgtggtaaaagtttgaatataatttacgacaggagctggcaagtgaaactccacaaaaaaagtaaatggctgcacacacgccattattttgaaaataagaagtactcacaaaatcagctggagctgccgcaagtgaaaaagaccagctgaagtcttattttaaactgtaaaatataaaaaaaaaaatagggcgtgaacaaaaatgagaaaataataccggatatgactattaagggcgtacactgaaactgggtaatatttgagaaaaagattaagataatagctgaacaaatgttgtgtgcagaacacggaacaatggtggcgaaaaaaaaaaacagtgtaagcacatggcgcgcacgtacttccgtgagaaaaattaaaaaaatttacccagtataaggtgcgtcattagacccgccttgtggcgcggttgtagccctgccctttgccccgccccgcgcgccgccccgcgcgccgcccccgccgccctcagccccgcccagcgccgccgcctccgcgacgcgctccgccccacagttacgtcagcacgccacgctcgccgtcgttgcgtcataaatgacgtggcaaaaatgattggcagttggaccgctgccatcagtgtactgtagattattgatgatg

SEQ ID NO: 2 - protein, 423 aa, Bovine Adenovirus
MASSRLIKEEMLDIVAPEIYKRKRPRRERAAPYAVKQEEKPLVKAERKIKRGSRKRALSGVDVPLPDDGFEDDEPHIEFVSAPRRPYQWKGRRVRRVLRPGVAVSFTPGARSLRPSSKRVYDEVYADDDFLEAAAAREGEFAYGKRGREAAQAQLLPAVAVPEPTYVVLDESNPTPSYKPVTEQKVILSRKRGVGKVEPTIQVLASKKRRMAENEDDRGAGSVAEVQMREVKPVTAALGIQTVDVSVPDHSTPMEVVQSLSRAAQVAQRLTQQQVRPSAKIKVEAMDLSAPVDAKPLDLKPVDVKPTPTFVLPSFRSLSTQTDSLPAAVVVPRKPRVHRATRRTARGLLPYYRLHPSITPTPGYRGSVYTSSGVRLPAVRRRRRRRTRRATPRLSAAAAAALLPGVRYHPSIRQAATVTRLRR

SEQ ID NO: 3 - DNA, 27 bp, Artificial Sequence
  Synthetic Construct
tctgctctgatgccgcatagttaagcc

SEQ ID NO: 4 - DNA, 41 bp, Artificial Sequence
  Synthetic Construct
cgctttctagagccgcggtaaatctcaggcgccacgatgtc

SEQ ID NO: 5 - DNA, 44 bp, Artificial Sequence
  Synthetic Construct
tcgtggcgcctgagatttaccgcggctctagaaagcgggccttg

SEQ ID NO: 6 - DNA, 36 bp, Artificial Sequence
  Synthetic Construct
aatactcgagagcgcttaacggcggagccgggttac

SEQ ID NO: 7 - DNA, 53 bp, Artificial Sequence
  Synthetic Construct
ctgcagcagctgctgcgggtgcagctcctgcgtaaatctcaggcgccacgatg

SEQ ID NO: 8 - DNA, 49 bp, Artificial Sequence
  Synthetic Construct
cgcaggagctgcacccgcagcagctgctgcagcaccgtatgctgtgaag

SEQ ID NO: 9 - DNA, 50 bp, Artificial Sequence
  Synthetic Construct
tttctagagccgcgagcagctgctgcctccgcctttactaaaggcttctc

SEQ ID NO: 10 - DNA, 51 bp, Artificial Sequence
  Synthetic Construct
ttagtaaaggcggaggcagcagctgctcgcggctctagaaagcgggccttg

SEQ ID NO: 11 - DNA, 40 bp, Artificial Sequence
  Synthetic Construct
ggagccgaattcatggcctcctctcggttgattaaagaag

SEQ ID NO: 12 - DNA, 42 bp, Artificial Sequence
  Synthetic Construct
cagcgctgaggcggggagtcgcgactgcaggcaggcgcacac

SEQ ID NO: 13 - DNA, 42 bp, Artificial Sequence
  Synthetic Construct
gtgtgcgcctgcctgcagtcgcgactccccgcctcagcgctg

SEQ ID NO: 14 - DNA, 25 bp, Artificial Sequence
  Synthetic Construct
gtccatggcgtgttaacaagctgtg

SEQ ID NO: 15 - protein, 413 aa, Artificial Sequence
  Synthetic Construct
MASSRLIKEEMLDIVAPEIYAGAAAPAAAAAPYAVKQEEKPLVKAERKIKRGSRKRALSGVDVPLPDDGFEDDEPHIEFVSAPRRPYQWKGRRVRRVLRPGVAVSFTPGARSLRPSSKRVYDEVYADDDFLEAAAAREGEFAYGKRGREAAQAQLLPAVAVPEPTYVVLDESNPTPSYKPVTEQKVILSRKRGVGKVEPTIQVLASKKRRMAENEDDRGAGSVAEVQMREVKPVTAALGIQTVDVSVPDHSTPMEVVQSLSRAAQVAQRLTQQQVRPSAKIKVEAMDLSAPVDAKPLDLKPVDVKPTPTFVLPSFRSLSTQTDSLPAAVVVPRKPRVHRATRRTARGLLPYYRLHPSITPTPGYRGSVYTSSGVRLPAVATPRLSAAAAAALLPGVRYHPSIRQAATVTRLRR

SEQ ID NO: 16 - protein, 24 aa, Artificial Sequence
  Synthetic Construct
MASSRLIKEEMLDIVAPEIYKRKR

SEQ ID NO: 17 - protein, 24 aa, Artificial Sequence
  Synthetic Construct
SRKRGVGKVEPTIQVLASKKRRMA

SEQ ID NO: 18 - protein, 30 aa, Bovine Adenovirus
KRKRPRRERAAPYAVKQEEKPLVKAERKIK

SEQ ID NO: 19 - protein, 21 aa, Bovine Adenovirus
RKRGVGKVEPTIQVLASKKRR

SEQ ID NO: 20 - protein, 10 aa, Bovine Adenovirus
RRRRRRRTRR

SEQ ID NO: 21 - protein, 4 aa, Artificial Sequence
  Synthetic Construct
  VARIANT 1, 2, 4: Xaa = Lys or Arg
  VARIANT 3: Xaa = Any Amino Acid
XXXX

SEQ ID NO: 22 - protein, 4 aa, Artificial Sequence
  Synthetic Construct
KRKR

SEQ ID NO: 23 - protein, 4 aa, Artificial Sequence
  Synthetic Construct
RRER

SEQ ID NO: 24 - protein, 4 aa, Artificial Sequence
  Synthetic Construct
RKIK

SEQ ID NO: 25 - DNA, 25 bp, Artificial Sequence
  Synthetic Construct
tgatccggtggccgacacaatcgag

SEQ ID NO: 26 - DNA, 48 bp, Artificial Sequence
  Synthetic Construct
tgtggccgcttggcggatgcctgcaggcacagtgggtttatcggcgcg

SEQ ID NO: 27 - DNA, 50 bp, Artificial Sequence
  Synthetic Construct
gccgataaacccactgtgcctgcaggcatccgccaagcggccacagtaac

SEQ ID NO: 28 - skipped

SEQ ID NO: 29 - protein, 30 aa, Artificial Sequence
  Synthetic Construct
AGAAPRRERAAPYAVKQEEKPLVKAERKIK

SEQ ID NO: 30 - protein, 30 aa, Artificial Sequence
  Synthetic Construct
KRKRPAAAAAAPYAVKQEEKPLVKAERKIK

SEQ ID NO: 31 - protein, 30 aa, Artificial Sequence
  Synthetic Construct
KRKRPRRERAAPYAVKQEEKPLVKAEAAAA

SEQ ID NO: 32 - protein, 30 aa, Artificial Sequence
  Synthetic Construct
AGAAPAAAAAAPYAVKQEEKPLVKAERKIK

SEQ ID NO: 33 - protein, 30 aa, Artificial Sequence
  Synthetic Construct
AGAAPRRERAAPYAVKQEEKPLVKAEAAAA

SEQ ID NO: 34 - protein, 30 aa, Artificial Sequence
  Synthetic Construct
KRKRPAAAAAAPYAVKQEEKPLVKAEAAAA

SEQ ID NO: 35 - protein, 30 aa, Artificial Sequence
  Synthetic Construct
AGAAPAAAAAAPYAVKQEEKPLVKAEAAAA

SEQ ID NO: 36 - protein, 31 aa, Artificial Sequence
  Synthetic Construct
MKRKRPRRERAAPYAVKQEEKPLVKAERKIK

SEQ ID NO: 37 - protein, 11 aa, Artificial Sequence
  Synthetic Construct
MRRRRRRRTRR